(19) United States
(12) Patent Application Publication
(10) Pub. No.: US 2008/0085884 A1
(43) Pub. Date: Apr. 10, 2008

(54) Melanin Concentrating Hormone Receptor-1 Antagonist Pyridinones

(75) Inventors: Duncan R. Armour, Sandwich (GB); Sebastian R. Galan, Sandwich (GB); Charlotte A. Lane, Sandwich (GB); Mark I. Lansdell, Sandwich (GB); Paul A. Stupple, Sandwich (GB)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 11/862,284

(22) Filed: Sep. 27, 2007

Related U.S. Application Data
(60) Provisional application No. 60/828,432, filed on Oct. 6, 2006.
(60) Provisional application No. 60/939,743, filed on May 23, 2007.

(51) Int. Cl. A61K 31/397; A61K 31/4427; A61K 31/4523; A61K 31/496; A61K 31/501; A61K 31/55; A61P 1/00; A61P 15/00; A61P 25/00; A61P 29/00; A61P 3/04; A61P 9/00; C07D 205/02; C07D 213/02; C07D 221/00; C07D 223/02; C07D 237/02; C07D 295/00

(52) U.S. Cl. 514/210.2; 514/217.04; 514/252.03; 514/253.01; 514/318; 514/333; 540/597; 544/238; 544/364; 546/194; 546/256

(57) ABSTRACT

The present invention provides for MCHR1 antagonist compounds of formula (I) 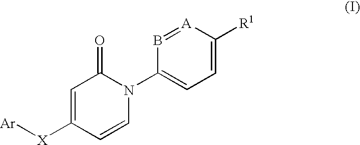 and the pharmaceutically acceptable salts, solvates and prodrugs thereof, wherein the substituents are as defined herein, and the pharmaceutically acceptable salts, solvates and prodrugs thereof, which are useful in treating diseases or conditions wherein antagonism of the MCHR1 receptor is beneficial.

The present invention relates to, inter alia, certain substituted pyridinone compounds, their salts, solvates and prodrugs, and their use in treating a variety of conditions. More particularly the compounds of interest are antagonists at the melanin concentrating hormone type 1 receptor (MCH-1 or MCHR1, and the like are terms used herein and which are interchangeable). As such the substances described herein are of use in the treatment of diseases or conditions mediated by MCHR1, such as obesity, and where antagonistic activity at this receptor would have a beneficial effect.

The hormone MCH, as found in humans, is a nonadecapeptide and is found throughout the central nervous system, as well as other tissues, including the gut, gonads, adipose tissue, pancreas, skin, and immune system. Recent reviews provide evidence that MCH is involved in many functions, including feeding, reproduction, stress, and other behavior patterns. (See, e.g., Griffon, B. & Baker, B. I., “Cell and Molecular Cell Biology of Melanin-Concentrating Hormone,” Int. Rev. Cytol., 213: 233-277 (2002); Kawano, H., et al., “Melanin-concentrating hormone neuron system: the wide web that controls the feeding,” Anatom. Sci. International, 77: 149-160 (2002) (disclosing effect of MCH on appetite, arousal and anxiety, food-searching behavior, olfaction, regulation of energy balance, swallowing and mastication); Borowsky, B., et al., “Antidepressant, anxiolytic and anorectic effects of a melanin-concentrating hormone-1 receptor antagonist,” Nature Med. 8: 825-830 (2002)). Antagonists of the MCH-1 receptor are being studied as a treatment for obesity and other eating disorder. (See, e.g., Crowley, V. E. F., et al, “Obesity Therapy: Altering the Energy Intake-and-Expenditure Balance Sheet,” Nature Reviews: Drug Discovery, 1: 26-286 (2002); Hillebrand, J. J. G., et al., “Neuropeptides, food intake and body weight regulation: a hypothalamic focus,” Peptides, 23: 2283-2306 (2002)); H J Dyke and N C Ray, Expert Opin. Ther. Patents (2005) 15(10): 1303-1313).

Preliminary investigations have indicated that the following diseases, conditions, and/or disorders are modulated by MCH receptor 1 antagonists: eating disorders (e.g., binge eating disorder, anorexia, and bulimia), weight loss or control (e.g., reduction in calorie or food intake, and/or appetite suppression), obesity, depression, atypical depression, bipolar disorders, psychoses, schizophrenia, behavioral addictions, suppression of reward-related behaviors (e.g., conditioned place avoidance, such as suppression of cocaine- and morphine-induced conditioned place preference), substance abuse, addictive disorders, impulsivity, alcoholism (e.g., alcohol abuse, addiction and/or dependence including treatment for abstinence, craving reduction and relapse prevention of alcohol intake), tobacco abuse (e.g., smoking addiction, cessation and/or dependence including treatment for craving reduction and relapse prevention of tobacco smoking), dementia (including memory loss, Alzheimers disease, dementia of aging, vascular dementia, mild cognitive impairment, age-related cognitive decline, and mild neurocognitive disorder), sexual dysfunction in males (e.g., erectile difficulty), seizure disorders, epilepsy, inflammation, gastrointestinal disorders (e.g., dysfunction of gastrointestinal motility or intestinal propulsion), attention deficit disorder (ADD including attention deficit hyperactivity disorder (ADHD)), Parkinson's disease, and type II diabetes.

Accordingly, it is an object of the invention to provide compounds of the present invention useful in treating diseases, conditions, or disorders that are modulated by MCH receptor 1 antagonists. Consequently, the compounds of the present invention (including the compositions and processes used therein) may be used in the manufacture of a medicament for the therapeutic applications described herein.

Another object of the invention is that the compounds of the invention have low inhibitory activity at the HERG potassium channel. Prolongation of the cardiac action potential duration (QT prolongation) has been identified as being due to action at the HERG potassium channel (Expert Opinion of Pharmacotherapy, 2, pp 947-973, 2000). QT prolongation is known to have a potential liability to produce fatal cardiac arrhythmias of Torsades de Pointes (TdP). In providing compounds which exhibit low inhibitory activity at the HERG potassium channel with comparable or improved pharmacokinetics, the invention aims to provide compounds which are therapeutically effective MCHR1 antagonists with good cardiac safety.

Other diseases, conditions and/or disorders for which MCH receptor 1 antagonists may be effective include: premenstrual syndrome or late luteal phase syndrome, migraines, panic disorder, anxiety, post-traumatic syndrome, social phobia, cognitive impairment in non-demented individuals, non-amnestic mild cognitive impairment, post operative cognitive decline, disorders associated with impulsive behaviours (such as, disruptive behaviour disorders (e.g., anxiety/depression, executive function improvement, tic disorders, conduct disorder and/or oppositional defiant disorder), adult personality disorders (e.g., borderline personality disorder and antisocial personality disorder), diseases associated with impulsive behaviours (e.g., substance abuse, paraphilias and self-mutilation), and impulse control disorders (e.g., intermittent explosive disorder, kleptomania, pyromania, pathological gambling, and trichotillomania)), obsessive compulsive disorder, chronic fatigue syndrome, premature ejaculation, sexual dysfunction in females, disorders of sleep (e.g., sleep apnea), autism, mutism, neurodegenerative movement disorders, spinal cord injury, damage of the central nervous system (e.g., trauma), stroke, neurodegenerative diseases or toxic or infective CNS diseases (e.g., encephalitis or meningitis), cardiovascular disorders (e.g., thrombosis).

Obesity is a major public health concern because of its increasing prevalence and associated health risks. Obesity and overweight are generally defined by body mass index (BMI), which is correlated with total body fat and estimates the relative risk of disease. BMI is calculated by weight in kilograms divided by height in meters squared (kg/m²). Overweight is typically defined as a BMI of 25-29.9 kg/m², and obesity is typically defined as a BMI of 30 kg/m² or more. See, e.g., National Heart, Lung, and Blood Institute, Clinical Guidelines on the Identification, Evaluation, and Treatment of Overweight and Obesity in Adults, The Evidence Report, Washington, D.C.: U.S. Department of Health and Human Services, NIH publication no. 98-4083 (1998). The increase in obesity is of concern because of the excessive health risks associated with obesity, including coronary heart disease, strokes, hypertension, type 2 diabetes mellitus, dyslipidemia, sleep apnea, osteoarthritis, gall bladder disease, depression, and certain forms of cancer (e.g., endometrial, breast, prostate, and colon). The negative health consequences of obesity make it the second leading cause of preventable death in the United States and impart a significant economic and psychosocial effect on society. See, McGinnis M, Foege W H., “Actual Causes of Death in the United States,” JAMA, 270, 2207-12 (1993).

Obesity is now recognized as a chronic disease that requires treatment to reduce its associated health risks. Although weight loss is an important treatment outcome, one of the main goals of obesity management is to improve cardiovascular and metabolic values to reduce obesity-related morbidity and mortality. It has been shown that 5-10% loss of body weight can substantially improve metabolic values, such as blood glucose, blood pressure, and lipid concentrations. Hence, it is believed that a 5-10% intentional reduction in body weight may reduce morbidity and mortality.

Currently available prescription drugs for managing obesity generally reduce weight by inducing satiety or decreasing dietary fat absorption. Satiety is achieved by increasing synaptic levels of norepinephrine, serotonin, or both. For example, stimulation of serotonin receptor subtypes 1B. 1D, and 2C and 1- and 2-adrenergic receptors decreases food intake by regulating satiety. See, Bray G A, “The New Era of Drug Treatment. Pharmacologic Treatment of Obesity: Symposium Overview,” Obes Res., 3(suppl 4), 415s-7s (1995). Adrenergic agents (e.g., diethylpropion, benzphetamine, phendimetrazine, mazindol, and phentermine) act by modulating central norepinephrine and dopamine receptors through the promotion of catecholamine release. Older adrenergic weight-loss drugs (e.g., amphetamine, methamphetamine, and phenmetrazine), which strongly engage in dopamine pathways, are no longer recommended because of the risk of their abuse. Fenfluramine and dexfenfluramine, both serotonergic agents used to regulate appetite, are no longer available for use. More recently, CB1 cannabinoid receptor antagonists/inverse agonists have been suggested as potential appetite suppressants. See, e.g., Arnone, M., et al., “Selective Inhibition of Sucrose and Ethanol Intake by SR141716, an Antagonist of Central Cannabinoid (CB1) Receptors,” Psychopharmacol, 132, 104-106 (1997); Colombo, G., et al., “Appetite Suppression and Weight Loss after the Cannabinoid Antagonist SR141716,” Life Sci., 63, PL113-PL117 (1998); Simiand, J., et al., “SR141716, a CB1 Cannabinoid Receptor Antagonist, Selectively Reduces Sweet Food Intake in Marmose,” Behav. Pharmacol., 9, 179-181 (1998); and Chaperon, F., et al., “Involvement of Central Cannabinoid (CB1) Receptors in the Establishment of Place Conditioning in Rats,” Psychopharmacology, 135, 324-332 (1998). For a review of cannabinoid CB1 and CB2 receptor modulators, see Pertwee, R. G., “Cannabinoid Receptor Ligands Clinical and Neuropharmacological Considerations, Relevant to Future Drug Discovery and Development,” Exp. Opin. Invest. Drugs, 9(7), 1553-1571 (2000).

Although investigations are on-going, there still exists a need for a more effective and safe therapeutic treatment for reducing or preventing weight-gain.

The present invention encompasses a method for promoting weight loss (including prevention or inhibition of weight gain), or treatment of obesity and related eating disorders which comprises the step of administering to an animal (preferably, human) in need thereof a therapeutically effective amount of a MCHR1 antagonist as described herein.

As used herein, “eating disorders” refer to illnesses in which the patient suffers disturbances in their eating behaviors and related thoughts and emotions. Representative examples of obesity-related eating disorders include overeating, bulimia, binge-eating disorder, compulsive dieting, nocturnal sleep-related eating disorder, pica, Prader-Willi Syndrome, and night-eating syndrome.

Bulimia (also referred to as Bulimia Nervosa) is characterized by self-perpetuating and self-defeating cycles of binge-eating and purging. A person binges by rapidly consuming a large amount of food (or what s/he perceives to be a large amount) in a discrete period of time and in an automatic and helpless manner.

Individuals with binge eating disorder (BED) binge eat but do not regularly use compensatory weight control behaviors such as vomiting, fasting, over-exercise, or abuse of laxatives. The person with BED is often genetically predisposed to weigh more than the “average” person, let alone the unrealistic cultural ideal. Due to culturally-reinforced body dissatisfaction, the person diets, making her or himself hungry, and then binges in response to that hunger. The person may also eat for emotional reasons: to comfort themselves, avoid uncomfortable situations, and numb feelings.

Symptoms of night-eating syndrome include: little or no appetite for breakfast; eating more food after dinner than during the meal; eating more than half of daily food intake after the dinner hour; the pattern persists for at least two months; feeling tense, anxious, upset, or guilty while eating; difficulty falling asleep or staying asleep; unlike bingeing (which is done in relatively short episodes). continual eating throughout evening hours; and eating produces guilt and shame, not enjoyment.

Unlike night-eating syndrome, a person suffering from nocturnal sleep-related eating disorder is somewhere between wakefulness and sleep, and may binge or consume strange combinations of food or non-food items. When awake, the person has little or no memory of the episodes.

Pica is a craving for non-food items, most commonly dirt, clay, chalk, paint chips, cornstarch, baking soda, coffee grounds, cigarette ashes, rust, plastic, etc. Pica is usually found in pregnant women, people whose diets are deficient in minerals contained in the consumed substances, people who have psychiatric disturbances, or people whose family or ethnic customs including eating certain non-food substances. Prader-Willi syndrome (PWS) is an uncommon inherited disorder characterized by mental retardation, decreased muscle tone, short stature, emotional lability and an insatiable appetite which can lead to life-threatening obesity.

The phrase “therapeutically effective amount” means an amount of a drug substance that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The terms “treating”, “treat”, or “treatment” embrace both preventative, i.e., prophylactic, and palliative treatment.

The term “animal” refers to humans (male or female, adults, adolescents and/or children), companion animals (e.g., dogs, cats and horses), food-source animals, zoo animals, marine animals, birds and other similar animal species. Preferred animals include humans, companion animals, and food-source animals, more preferably, humans.

Various pyridinones and pyrimidinones and MCH antagonists are reported in the art, notably:

WO2003068230 (Pharmacia)—compounds disclosed for the treatment of conditions caused by unregulated p38 MAP kinase or TNF activity such as inflammation or arthritis etc.; WO2003076405 (Bayer)—compounds for the treatment of COPD etc.; WO2003097047 (Eli Lilly)—MCH antagonists for treatment of conditions such as obesity; WO2004052848 (Eli Lilly)—MCH antagonists for treatment of conditions such as obesity; WO2004072025 (Aventis)—MCH antagonists for treating conditions such as obesity; WO2005018557 (Pharmacia)—compounds disclosed for the treatment of conditions caused by unregulated p38 MAP kinase or TNF activity such as inflammation or arthritis etc.; WO2005042541 (Glaxo)—thienopyrimid-4-one derivatives useful as MCHR1 antagonists for treating conditions such as obesity; WO2005070925 (Aventis)—MCH antagonists for the treating conditions such as obesity; WO2005103039 (Neurocrine Bioscience)—MCHR1 antagonists for treating conditions such as obesity; EP481-448 (Squibb & Sons)—Compounds disclosed as antihyperintensive agents; U.S. Pat. No. 5,470,975 (Squibb & Sons)—Compounds disclosed as antihyperintensive agents; WO0121577 (Takeda)—MCH antagonists for the treatment of obesity etc.; WO2001082925 (Takeda)—MCH antagonists for the treatment of obesity etc.; US20020183324 (Bristol-Myers Squibb)—Compounds disclosed as factor Xa inhibitors for use as anticoagulants; WO2003026652 (Bristol-Myers Squibb)—Compounds disclosed as factor Xa inhibitors for use as anticoagulants; WO2003059884 (X-Ceptor)—Compounds disclosed as liver X receptor modulators for a wide range of diseases; US20040006062 (Bristol-Myers Squibb)—Compounds disclosed as factor Xa inhibitors for use as anticoagulants; US20040132718 (Bristol-Myers Squibb)—Compounds disclosed as factor Xa inhibitors for use as anticoagulants; US20040220174 (Bristol-Myers Squibb)—Compounds disclosed as factor Xa inhibitors for use as anticoagulants; WO2005032472 (Bristol-Myers Squibb)—Compounds disclosed as factor Xa inhibitors for use as anticoagulants; US20050267097 (Bristol-Myers Squibb)—Compounds disclosed as factor Xa inhibitors for use as anticoagulants; WO20060565708 (Institutes for Pharmaceutical Discovery)—protein tyrosine phosphatases for the treatment of diabetes.

The present invention provides for compounds of formula (I) below 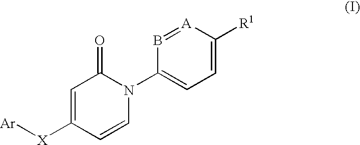
wherein
X is CH₂CH₂, CH₂O or OCH₂;
A and B are each independently CH or N, with the proviso that 1 or both of A and B is N;
Ar is phenyl optionally substituted by 1 or 2 substituents independently selected from F and Cl;
R¹ is a saturated 4- to 9-membered heterocyclic ring system containing 1 or 2 ring N atoms, which ring system may incorporate spiro-, fused or bridged rings,
which is attached to the “ABCCHCHC” ring via a N atom,
which ring system is optionally substituted by one or more substituents independently selected from ═O, R⁹, OH, C(O)C₁-C₅ alkyl C(O)C₃-C₅ cycloalkyl, C(O)OC₁-C₅ alkyl NR⁶R⁷, NR⁸C(O)R⁹, NR⁸C(O)OR⁹, O(C₁-C₅ alkyl) or O(C₃-C₅ cycloalkyl);
R⁶ and R⁷ are each independently H, C₁-C₅ alkyl or C₃-C₅ cycloalkyl;
or R⁶ and R⁷ can be taken together with the N atom to which they are attached to form a 4- to 7-membered saturated ring, optionally substituted by ═O;
R⁸ is H, C₁-C₅ alkyl or C₃-C₅ cycloalkyl;
R⁹ is C₁-C₅ alkyl or C₃-C₅ cycloalkyl, each of which is optionally substituted with one or more fluorine atoms;
and the pharmaceutically acceptable salts, solvates and prodrugs thereof.

“Alkyl” may be either straight chain or branched.

“Me” is methyl, and “Et” is ethyl.

Suitable 5-membered aromatic heterocycles include oxazole, isoxazole, imidazole, pyrazole, thiazole, isothiazole and oxadiazole. Suitable 6-membered aromatic heterocycles include pyridine, pyridazine, pyrimidine, pyrazine and triazine.

The pharmaceutically acceptable salts of the compounds of the formula (I) include the acid addition and the base salts thereof. A pharmaceutically acceptable salt of a compound of the formula (I) may be readily prepared by mixing together solutions of a compound of the formula (I) and the desired acid or base, as appropriate. The salt may precipitate from solution and be collected by filtration or may be recovered by evaporation of the solvent. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. For a review on suitable salts, see Handbook of Pharmaceutical Salts Properties, Selection, and Use by Stahl and Wermuth (Wiley-VCH, 2002).

The compounds and salts of the present invention may inherently form solvates with pharmaceutically acceptable solvents (including water) and it is intended that the invention embrace both solvated and unsolvated forms. The term “solvate” refers to a complex of a compound or salt of the present invention with one or more solvent molecules.

As indicated, so-called ‘prodrugs’ of the compounds of formula I are also within the scope of the invention. Thus certain derivatives of compounds of formula I which may have little or no pharmacological activity themselves can, when administered into or onto the body, be converted into compounds of formula I having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (Ed. E. B. Roche, American Pharmaceutical Association).

Prodrugs in accordance with the invention can, for example, be produced by replacing appropriate functionalities present in the compounds of formula I with certain moieties known to those skilled in the art as ‘pro-moieties’ as described, for example, in Design of Prodrugs by H. Bundgaard (Elsevier, 1985).

Some examples of prodrugs in accordance with the invention include:
(a) where the compound of formula I contains an alcohol functionality (—OH), an ether thereof, for example, a compound wherein the hydrogen of the alcohol functionality of the compound of formula I is replaced by (C₁-C₆)alkanoyloxymethyl; and
(b) where the compound of formula I contains a primary or secondary amino functionality (—NH₂ or —NHR where R≠H), an amide thereof, for example, a compound wherein, as the case may be, one or both hydrogens of the amino functionality of the compound of formula I is/are replaced by (C₁-C₁₀)alkanoyl.

Further examples of replacement groups in accordance with the foregoing examples and examples of other prodrug types may be found in the aforementioned references.

Moreover, certain compounds of formula I may themselves act as prodrugs of other compounds of formula I.

Certain compounds of formula (I) may have asymmetric centers and therefore exist in different enantiomeric forms. All optical isomers and stereoisomers of the compounds of formula (I), and mixtures thereof, are considered to be within the scope of the invention. Diastereomeric mixtures can be separated into their individual diastereomers on the basis of their physical chemical differences by methods known to those skilled in the art, for example, by chromatography or fractional crystallization. Enantiomers can be separated by converting the enantiomeric mixtures into a diastereomeric mixture by reaction with an appropriate optically active compound (e.g., alcohol), separating the diastereomers and converting (e.g., hydrolyzing) the individual diastereomers to the corresponding pure enantiomers. With respect to the compounds of formula (I), the invention includes the use of a racemate, one or more enantiomeric forms, one or more diastereomeric forms, or mixtures thereof. The compounds of formula (I) may also exist as tautomers. This invention relates to the use of all such tautomers and mixtures thereof.

The compounds described herein, including the pharmaceutically acceptable salts of such compounds, also include isotopically-labelled compounds, which are identical to those recited in Formula (I), but for the fact that one or more atoms are replaced by an atom having an atomic mass or mass number different from the atomic mass or mass number usually found in nature. Examples of isotopes that can be incorporated into compounds of the invention include isotopes of H, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e. ²H, can afford certain therapeutic advantages resulting from greater metabolic stability, for example increased in vivo half-life or reduced dosage requirements and, hence, may be preferred in some circumstances. Isotopically labelled compounds of Formula (I) of this invention and prodrugs thereof can generally be prepared by carrying out the procedures disclosed in the Schemes and/or in the Examples and Preparations below, by substituting a readily available isotopically labelled reagent for a non-isotopically labelled reagent.

In the case of agents that are solids, it is understood by those skilled in the art that the inventive compounds and salts may exist in different crystal or polymorphic forms, all of which are intended to be within the scope of the present disclosure and specified formulas.

A “pharmacological composition” refers to a mixture of one or more of the compounds described herein, or physiologically acceptable salts and solvates thereof, with other chemical components, such as physiologically acceptable carriers and/or excipients.

The purpose of a pharmacological composition is to facilitate administration of a compound to an organism.

A “physiologically acceptable carrier” refers to a carrier or diluent that does not cause significant or otherwise unacceptable irritation to an organism and does not unacceptably abrogate the biological activity and properties of the administered compound.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefor, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Once improvement of the patient's conditions has occurred, a maintenance dose is administered if necessary. Subsequently, the dosage or the frequency of administration, or both, can be reduced, as a function of the symptoms, to a level at which the improved proliferative disorder or condition is retained. When the symptoms have been alleviated to the desired level, treatment can cease. Patients can, however, require intermittent treatment on a long-term basis upon any recurrence of the disease symptoms.

The amount and frequency of administration of the compounds used in the methods described herein and, if applicable, other agents will be regulated according to the judgment of the attending clinician (physician) considering such factors as age, condition and size of the patient as well as severity of the disease being treated.

The amount of the active compound administered (e.g., for treatment, prophylactic, and/or maintenance) will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to about 7 g/day, preferably about 0.2 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day.

The compounds of this invention may also be used in conjunction with other pharmaceutical agents for the treatment of the diseases, conditions and/or disorders described herein. Therefore, methods of treatment that include administering compounds of the present invention in combination with other pharmaceutical agents are also provided. Suitable pharmaceutical agents that may be used in combination with the compounds of the present invention include anti-obesity agents such as apolipoprotein-B secretion/microsomal triglyceride transfer protein (apo-B/MTP) inhibitors, 11β-hydroxy steroid dehydrogenase-1 (11β-HSD type 1) inhibitors, peptide YY₃₋₃₆ or analogs thereof, cannabinoid antagonists (e.g., CB-1 antagonists, such as rimonabant), MCR-4 agonists, cholecystokinin-A (CCK-A) agonists, monoamine reuptake inhibitors (such as sibutramine), sympathomimetic agents, neurotensin inhibitors, β₃ adrenergic receptor agonists, dopamine agonists (such as bromocriptine), melanocyte-stimulating hormone receptor analogs, 5HT2c agonists (e.g. APD356 (Arena)), leptin (the OB protein), leptin analogs, leptin receptor agonists, galanin antagonists, lipase inhibitors (such as tetrahydrolipstatin, i.e. orlistat), anorectic agents (such as a bombesin agonist), Neuropeptide-Y receptor antagonists (e.g., NPY Y5 receptor antagonists, such as the spiro compounds described in U.S. Pat. Nos. 6,566,367; 6,649,624; 6,638,942; 6,605,720; 6,495,559; 6,462,053; 6,388,077; 6,335,345; and 6,326,375; US Publication Nos. 2002/0151456 and 2003/036652; and PCT Publication Nos. WO 03/010175. WO 03/082190 and WO 02/048152), thyromimetic agents, dehydroepiandrosterone or an analog thereof, glucocorticoid receptor agonists or antagonists, orexin receptor antagonists, glucagon-like peptide-1 receptor agonists, ciliary neurotrophic factors (such as Axokine™ available from Regeneron Pharmaceuticals, Inc., Tarrytown, N.Y. and Procter & Gamble Company, Cincinnati, Ohio), human agouti-related proteins (AGRP), ghrelin receptor antagonists, histamine 3 receptor antagonists or inverse agonists, neuromedin U receptor agonists; stearoyl Co-A desaturase 1 (SCD1) ligands; 5HT6 antagonists; diacylglycerol acyl transferase 1 (DGAT1) inhibitors; diacylglycerol acyl transferase 2 (DGAT2) inhibitors; DPPIV inhibitors; acetyl CoA carboxylase (ACC) inhibitors; sodium dependent glucose transporter 2 (SGLT2) inhibitors; PPAR modulators (alpha, beta, gamma); PDE-10 inhibitors (see e.g. International Patent Application publication WO 2005/120514); opioid receptor antagonists (mu, kappa and delta); zonisamide; topiramate; radaxafine; and the like. Other anti-obesity agents, including the preferred agents set forth hereinbelow, are well known, or will be readily apparent in light of the instant disclosure, to one of ordinary skill in the art.

Especially preferred are anti-obesity agents selected from the group consisting of orlistat, sibutramine, bromocriptine, ephedrine, leptin, pseudoephedrine; rimonabant, peptide YY₃₋₃₆ or an analog thereof; and 2-oxo-N-(5-phenylpyrazinyl)spiro-[isobenzofuran-1(3H), 4′-piperidine]-1′-carboxamide. Preferably, compounds of the present invention and combination therapies are administered in conjunction with exercise and a sensible diet.

Representative anti-obesity agents for use in the combinations, pharmaceutical compositions, and methods of the invention can be prepared using methods known to one of ordinary skill in the art, for example, sibutramine can be prepared as described in U.S. Pat. No. 4,929,629; bromocriptine can be prepared as described in U.S. Pat. Nos. 3,752,814 and 3,752,888; orlistat can be prepared as described in U.S. Pat. Nos. 5,274,143; 5,420,305; 5,540,917; and 5,643,874; rimonabant can be prepared as described in U.S. Pat. No. 5,624,941; PYY₃₋₃₆ (including analogs) can be prepared as described in US Publication No. 2002/0141985 and WO 03/027637; and the NPY Y5 receptor antagonist 2-oxo-N-(5-phenylpyrazinyl)spiro[isobenzofuran-1(3H), 4′-piperidine]-1′-carboxamide can be prepared as described in US Publication No. 2002/0151456. Other useful NPY Y5 receptor antagonists include those described in PCT Publication No. 03/082190, such as 3-oxo-N-(5-phenyl-2-pyrazinyl)spiro[isobenzofuran-1(3H), 4′-piperidine]-1′-carboxamide; 3-oxo-N-(7-trifluoromethylpyrido[3,2-b]pyridin-2-yl)-spiro-[isobenzofuran-1(3H), 4′-piperidine]-1′-carboxamide; N-[5-(3-fluorophenyl)-2-pyrimidinyl]-3-oxospiro-[isobenzofuran-1(3H), [4′-piperidine]-1′-carboxamide; trans-3′-oxo-N-(5-phenyl-2-pyrimidinyl)]spiro[cyclohexane-1,1′(3′H)-isobenzofuran]-4-carboxamide; trans-3′-oxo-N-[1-(3-quinolyl)-4-imidazolyl]spiro[cyclohexane-1,1′(3′H)-isobenzofuran]-4-carboxamide; trans-3-oxo-N-(5-phenyl-2-pyrazinyl)spiro[4-azaiso-benzofuran-1(3H), 1′-cyclohexane]-4′-carboxamide; trans-N-[5-(3-fluorophenyl)-2-pyrimidinyl]-3-oxospiro[5-azaisobenzofuran-1(3H), 1′-cyclohexane]-4′-carboxamide; trans-N-[5-(2-fluorophenyl)-2-pyrimidinyl]-3-oxospiro[5-azaisobenzofuran-1(3H), 1′-cyclohexane]-4′-carboxamide; trans-N-[1-(3,5-difluorophenyl)-4-imidazolyl]-3-oxospiro[7-azaisobenzofuran-1(3H), 1′-cyclohexane]-4′-carboxamide; trans-3-oxo-N-(1-phenyl-4-pyrazolyl)spiro[4-azaisobenzofuran-1(3H), 1′-cyclohexane]-4′-carboxamide; trans-N-[1-(2-fluorophenyl)-3-pyrazolyl]-3-oxospiro[6-azaisobenzofuran-1(3H), 1′-cyclohexane]-4′-carboxamide; trans-3-oxo-N-(1-phenyl-3-pyrazolyl)spiro[6-azaisobenzofuran-1(3H), 1′-cyclohexane]-4′-carboxamide; trans-3-oxo-N-(2-phenyl-1,2,3-triazol-4-yl)spiro[6-azaisobenzofuran-1(3H), 1′-cyclohexane]-4′-carboxamide; and pharmaceutically acceptable salts and esters thereof. All of the above recited U.S. patents and publications are incorporated herein by reference.

Other suitable pharmaceutical agents that may be administered in combination with the compounds of the present invention include agents designed to treat tobacco abuse (e.g., nicotine receptor partial agonists, bupropion hypochloride (also known under the tradename Zyban™) and nicotine replacement therapies), agents to treat erectile dysfunction (e.g., dopaminergic agents, such as apomorphine), ADD/ADHD agents (e.g., Ritalin™, Strattera™, Concerta™ and Adderall™), and agents to treat alcoholism, such as opioid antagonists (e.g., naltrexone (also known under the tradename ReVia™) and nalmefene), disulfiram (also known under the tradename Antabuse™), and acamprosate (also known under the tradename Campral™)). In addition, agents for reducing alcohol withdrawal symptoms may also be co-administered, such as benzodiazepines, beta-blockers, clonidine, carbamazepine, pregabalin, and gabapentin (Neurontin™). Treatment for alcoholism is preferably administered in combination with behavioral therapy including such components as motivational enhancement therapy, cognitive behavioral therapy, and referral to self-help groups, including Alcohol Anonymous (AA).

Other pharmaceutical agents that may be useful include antihypertensive agents; anti-inflammatory agents (e.g., COX-2 inhibitors); antidepressants (e.g., fluoxetine hydrochloride (Prozac™)); cognitive improvement agents (e.g., donepezil hydrochloride (Aircept™) and other acetylcholinesterase inhibitors); neuroprotective agents (e.g., memantine); antipsychotic medications (e.g., ziprasidone (Geodon™), risperidone (Risperdal™), and olanzapine (Zyprexa™)); insulin and insulin analogs (e.g., LysPro insulin); GLP-1 (7-37) (insulinotropin) and GLP-1 (7-36)-NH₂; sulfonylureas and analogs thereof: chlorpropamide, glibenclamide, tolbutamide, tolazamide, acetohexamide, Glypizide®, glimepiride, repaglinide, meglitinide; biguanides: metformin, phenformin, buformin; α-2-antagonists and imidazolines: midaglizole, isaglidole, deriglidole, idazoxan, efaroxan, fluparoxan; other insulin secretagogues: linogliride, A-4166; glitazones: ciglitazone, Actos® (pioglitazone), englitazone, troglitazone, darglitazone, Avandia® (BRL49653); fatty acid oxidation inhibitors: clomoxir, etomoxir; α-glucosidase inhibitors: acarbose, miglitol, emiglitate, voglibose, MDL-25,637, camiglibose, MDL-73,945; 1-agonists: BRL 35135, BRL 37344, RO 16-8714, ICI D7114, CL 316,243; phosphodiesterase inhibitors: L-386,398; lipid-lowering agents: benfluorex: fenfluramine; vanadate and vanadium complexes (e.g., Naglivan®) and peroxovanadium complexes; amylin antagonists; glucagon antagonists; gluconeogenesis inhibitors; somatostatin analogs; antilipolytic agents: nicotinic acid, acipimox, WAG 994, pramlintide (Symlin™), AC 2993, nateglinide, aldose reductase inhibitors (e.g., zopolrestat), glycogen phosphorylase inhibitors, sorbitol dehydrogenase inhibitors, sodium-hydrogen exchanger type 1 (NHE-1) inhibitors and/or cholesterol biosynthesis inhibitors or cholesterol absorption inhibitors, especially a HMG-CoA reductase inhibitor (e.g., atorvastatin or the hemicalcium salt thereof), or a HMG-CoA synthase inhibitor, or a HMG-CoA reductase or synthase gene expression inhibitor, a CETP inhibitor, a bile acid sequesterant, a fibrate, an ACAT inhibitor, a squalene synthetase inhibitor, an anti-oxidant or niacin. The compounds of the present invention may also be administered in combination with a naturally occurring compound that acts to lower plasma cholesterol levels. Such naturally occurring compounds are commonly called nutraceuticals and include, for example, garlic extract, Hoodia plant extracts, and niacin.

Examples of anti-diabetes agents suitable for combination therapy with the present invention include, by way of example only: Biguanides, Glucophage Tablets (Bristol-Myers-Squibb); Glucosadase Inhibitors, for example, Precose Tablets (Bayer); Intermediate Acting Insulins, for example, Humulin L (Lilly), Humulin N (Lilly), Humulin N NPH (Lilly), Iletin II (Lilly), Iletin II NPH (pork) (Lilly); Novolin L Human Insulin (Novo Nordisk); Novolin N Human Insulin (Novo Nordisk); Novolin N PenFill (Novo Nordisk); Novolin N prefilled Syringe Disposable Insulin Delivery System (Novo Nordisk); Purified Pork Lente Insulin (Novo Nordisk); Purified Pork NPH Isophane Insulin (Novo Nordisk).

Examples of intermediate and rapid acting insulins suitable for combination therapy with the present invention include, by way of example only: Humulin 50/50 (Lilly); Humulin 70/30 (Lilly); Humulin 70/30 cartridge (Lilly); Novolin 70/30 PenFill (Novo Nordisk); Novolin 70/30 Prefilled Disposable Insulin Delivery system (Novo Nordisk). Examples of rapid acting insulins suitable for combination therapy with the present invention include, by way of example only: Humalog Injection (Lilly); Humulin R Regular (Lilly); Iletin II (Lilly), Regular (pork); Novolin R Human Insulin (Novo Nordisk); Purified Pork Regular Insulin (Novo Nordisk); Velosulin BR Human Insulin (Novo Nordisk).

Further examples of other anti-diabetes agents suitable for combination therapy with the present invention include, by way of example only meglitinides, sulfonylureas, and thiazolidinediones. Examples of meglitinides include, but are not limited to: Prandin Tablets (Novo Nordisk. Examples of sulfonylureas include, but are not limited to Amaryl tablets (Hoechst Marion Roussel), Diabeta tablets (Hoechst Marion Roussel), Diabinese Tablets (Pfizer), Glucotrol Tablets (Pfizer), Glucotrol XL Extended Release Tablets (Pfizer), Glynase PresTab Tablets (Pharmacia & Upjohn), Micronase Tablets (Parke-Davis). Examples of thiazolidinediones include, but are not limited to Rezulin Tablets (Parke-Davis).

When depression is of concern (e.g., treatment of, prevention of, maintenance against, etc.), the patient would be expected to benefit from administration of a compound of Formula I in conjunction with, by way of example only, Effexor Tablets (Wyeth); Effexor XR Capsules (Wyeth); Remeron Tablets (Organon); Remeron SolTab Tablets (Organon); Serzone Tablets (Bristol-Myers Squibb); Wellbutrin Tablets (GlaxoSmithKline); Wellbutrin SR Sustained-Release Tablets (GlaxoSmithKline); Nardil Tablets (Parke-Davis); Parnate Tablets (GlaxoSmithKline); Selective Serotonin Reuptake Inhibitors (SSRI) Celexa Oral Solution (Forest); Celexa Tablets (Forest); Lexapro Tablets (Forest); Paxil CR Controlled-Release Tablets (GlaxoSmithKline); Paxil Oral Suspension (GlaxoSmithKline); Paxil Tablets (GlaxoSmithKline); Prozac Pulvates and Liquid (Dista); Zoloft Oral Concentrate (Pfizer); Zoloft Tablets (Pfizer); Norpramin Tablets (Aventis); Sinequan Capsules (Pfizer); Sinequan Oral Concentrate (Pfizer); Surmontil Capsules (Odyssey); Vivactil Tablets (Merck and Odyssey).

Examples of anxiolytic agents suitable for combination therapy with the present invention include, by way of example only: Tranxene T-TAB tablets (Abbott); Tranxene-SD Tablets (Abbott); Tranxene SD Half Strength Tablets (Abbott); Valium Tablets (Roche Products); Xanax Tablets (Pharmacia & Upjohn); Atarax Tablets & Syrup (Pfizer); Effexor XR Capsules (Wyeth); Paxil Oral Suspension (GlaxoSmithKline); Paxil Tablets (GlaxoSmithKline); Sinequan Capsules (Pfizer); Sinequan Oral Concentrate (Pfizer); Vistaril Capsules (Pfizer); Vistaril Intramuscular Solution (Pfizer); Vistaril Oral Suspension (Pfizer); Zoloft Oral Concentrate (Pfizer); Zoloft Tablets (Pfizer).

In some situations, centrally-acting compounds, when either administered alone or in combination therapies such as those mentioned herein, may cause nausea and/or emesis and so it could be advantageous to administer compounds or combinations of the present invention alongside a suitable anti-emetic agent, for example a 5-HT₃ antagonist or a neurokinin-1 (NK-1) antagonist.

Suitable 5-HT₃ antagonists include, but are not limited to, granisetron, ondansetron, tropisetron, ramosetron, palonsetron, indisetron, dolasetron, alosetron and azasetron.

Suitable NK-1 antagonists include, but are not limited to, aprepitant, casopitant, ezlopitant, cilapitant, netupitant, vestipitant, vofopitant and 2-(R)-(1-(R)-3,5-bis(trifluoromethyl)phenyl)ethoxy-4-(5-(dimethylamino)methyl-1,2,3-triazol-4-yl)methyl-3-(S)-(4-fluorophenyl)morpholine. See for example International Patent Application publication number WO2006/049933.

The dosage of the additional pharmaceutical agent (e.g., anti-obesity agent) will also be generally dependent upon a number of factors including the health of the subject being treated, the extent of treatment desired, the nature and kind of concurrent therapy, if any, and the frequency of treatment and the nature of the effect desired. In general, the dosage range of an anti-obesity agent is in the range of from about 0.001 mg to about 100 mg per kilogram body weight of the individual per day, preferably from about 0.1 mg to about 10 mg per kilogram body weight of the individual per day. However, some variability in the general dosage range may also be required depending upon the age and weight of the subject being treated, the intended route of administration, the particular anti-obesity agent being administered and the like. The determination of dosage ranges and optimal dosages for a particular patient is also well within the ability of one of ordinary skill in the art having the benefit of the instant disclosure.

In general, the compounds described herein and, in embodiments where combinational therapy is employed, other agents do not have to be administered in the same pharmaceutical composition, and may, because of different physical and chemical characteristics, have to be administered by different routes. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician. The particular choice of compounds used will depend upon the diagnosis of the attending physicians and their judgment of the condition of the patient and the appropriate treatment protocol. The compounds may be administered concurrently (e.g., simultaneously, essentially simultaneously or within the same treatment protocol) or sequentially, depending upon the nature of the proliferative disease, the condition of the patient, and the actual choice of compounds used. By sequentially and within the same treatment protocol is meant that the active agents may be administered in any order, as deemed appropriate by the skilled clinician, including for example, a first administration of active agent A, followed by an administration of active agent B, which could be repeated on a daily or other timeframe. Alternatively, active agent A could be administered for a period (e.g. a few days/weeks), and the treatment switched to administration of active agent B for a further period. Active agents A and B could be single agents or a combination of agents, where at least one of A and B comprises a compound of formula I, or a salt, solvate or prodrug thereof according to the invention.

Pharmaceutical Compositions/Formulations, Dosaging, and Modes of Administration

Pharmaceutical (including pharmaceuticals for veterinarial use) compositions according to the invention may, alternatively or in addition to a compound of Formula (I), comprise as an active ingredient or pharmaceutically acceptable salts of such compounds. Such compounds and salts are sometimes referred to herein collectively as “active agents” or “agents.” Administration of the compounds of the present invention (hereinafter the “active compound(s)”) can be effected by any method that enables delivery of the compounds to the site of action.

Methods of preparing various pharmaceutical compositions with a specific amount of active compound are known, or will be apparent, to those skilled in this art. In addition, those of ordinary skill in the art are familiar with formulation and administration techniques. Such topics are discussed, e.g., in Goodman and Gilman's The Pharmacological Basis of Therapeutics, current edition, Pergamon Press; and Remington's Pharmaceutical Sciences (current edition.) Mack Publishing Co., Easton, Pa. These techniques can be employed in appropriate aspects and embodiments of the methods and compositions described herein. The following examples are provided for illustrative purposes only and are not meant to serve as limitations of the present disclosure.

The compounds utilized in the methods of the instant invention may be administered either alone or in combination with pharmaceutically acceptable carriers, excipients or diluents, in a pharmaceutical composition, according to standard pharmaceutical practice.

Administration methods include oral routes (e.g. with a solid or liquid formulation), intraduodenal routes, parenteral injection (including intravenous, subcutaneous, intramuscular, intravascular or infusion), topical, pulmonary, intranasal, and rectal administration. For example, the therapeutic or pharmaceutical compositions of the invention can be administered locally to the area in need of treatment. This may be achieved by, for example, but not limited to, local infusion during surgery, topical application, e.g., cream, ointment, injection, catheter, or implant, said implant made, e.g., out of a porous, non-porous, or gelatinous material, including membranes, such as sialastic membranes, or fibers. The administration can also be by direct injection at the site (or former site) of a tumor or neoplastic or pre-neoplastic tissue.

Still further, the therapeutic or pharmaceutical composition can be delivered in a vesicle, e.g., a liposome (see, for example, Langer, 1990, Science, 249:1527-1533; Treat et al., 1989, Liposomes in the Therapy of Infectious Disease and Cancer, Lopez-Bernstein and Fidler (eds.), Liss, N.Y., pp. 353-365).

The pharmaceutical compositions used in the methods of the present invention can be delivered in a controlled release system. In one embodiment, a pump may be used (see, Sefton, 1987, CRC Crit. Ref. Biomed. Eng. 14:201; Buchwald et al., 1980, Surgery, 88:507; Saudek et al., 1989, N. Engl. J. Med., 321:574). Additionally, a controlled release system can be placed in proximity of the therapeutic target (see, Goodson, 1984, Medical Applications of Controlled Release, Vol. 2, pp. 115-138).

The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

The pharmaceutical compositions used in the methods of the instant invention can contain the active ingredient in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be, for example, inert diluents, such as calcium carbonate, sodium carbonate, lactose, calcium phosphate or sodium phosphate; granulating and disintegrating agents, such as microcrystalline cellulose, sodium crosscarmellose, corn starch, or alginic acid; binding agents, for example starch, gelatin, polyvinylpyrrolidone or acacia, and lubricating agents, for example, magnesium stearate, stearic acid or talc. The tablets may be uncoated or they may be coated by known techniques to mask the taste of the drug or delay disintegration and absorption in the gastrointestinal tract and thereby provide a sustained action over a longer period. For example, a water soluble taste masking material such as hydroxypropylmethylcellulose or hydroxypropylcellulose, or a time delay material such as ethyl cellulose, or cellulose acetate butyrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

For companion animals or food-source animals, it is often more convenient to incorporate the drug substance into the animal's food source. This may be accomplished by addition of the drug substance to the food source as a dry powder or as a liquid solution or suspension.

Aqueous suspensions can contain the active material in admixture with excipients suitable for the manufacture of aqueous suspensions. Such excipients can act as suspending agents and include, e.g., sodium carboxymethylcellulose, methylcellulose, hydroxypropylmethyl-cellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant, e.g., butylated hydroxyanisol, alpha-tocopherol, or ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of antioxidant(s).

The pharmaceutical compositions used in the methods of the instant invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

Pulmonary administration by inhalation may be accomplished by means of producing liquid or powdered aerosols, for example, by using any of various devices known in the art (see e.g. Newman, S. P., 1984, in Aerosols and the Lung, Clarke and Pavia (Eds.), Butterworths, London, England, pp. 197-224; PCT Publication No. WO 92/16192 dated Oct. 1, 1992; PCT Publication No. WO 91/08760 dated Jun. 27, 1991; NTIS Patent Application 7-504-047 filed Apr. 3, 1990 by Roosdorp and Crystal) including but not limited to nebulizers, metered dose inhalers, and powder inhalers. Various delivery devices are commercially available and can be employed, e.g. Ultravent nebulizer (Mallinckrodt, Inc, St. Louis, Mo.); Acorn II nebulizer (Marquest Medical Products, Englewood, Colo.); Ventolin metered dose inhalers (Glaxo Inc., Research Triangle Park, N.C.); Spinhaler powder inhaler (Fisons Corp., Bedford, Mass.) or Turbohaler (Astra). Such devices typically entail the use of formulations suitable for dispensing from such a device, in which a propellant material may be present. Ultrasonic nebulizers may also be used.

A nebulizer may be used to produce aerosol particles, or any of various physiologically inert gases may be used as an aerosolizing agent. Other components such as physiologically acceptable surfactants (e.g. glycerides), excipients (e.g. lactose), carriers (e.g. water, alcohol), and diluents may also be included.

As will be understood by those skilled in the art of delivering pharmaceuticals by the pulmonary route, a major criteria for the selection of a particular device for producing an aerosol is the size of the resultant aerosol particles. Smaller particles are needed if the drug particles are mainly or only intended to be delivered to the peripheral lung, i.e. the alveoli (e.g. 0.1-3 μm), while larger drug particles are needed (e.g. 3-10 μm) if delivery is only or mainly to the central pulmonary system such as the upper bronchi. Impact of particle sizes on the site of deposition within the respiratory tract is generally known to those skilled in the art.

The pharmaceutical compositions may be in the form of a sterile injectable aqueous solutions. Among the acceptable vehicles and solvents that may be employed are water, Ringers solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulsion.

The injectable solutions or microemulsions may be introduced into a patient's blood-stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents, which have been mentioned above. The sterile injectable preparation may also be a sterile injectable solution or suspension in a non-toxic parenterally-acceptable diluent or solvent, for example as a solution in 1,3-butanediol. Exemplary parenteral administration forms also include solutions or suspensions of active compounds in sterile aqueous solutions, for example, aqueous propylene glycol or dextrose solutions. All such dosage forms can be suitable buffered, if desired. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of Formula I may also be administered in the form of suppositories for rectal administration of the drug. These compositions can be prepared by mixing the inhibitors with a suitable non-irritating excipient, which is solid at ordinary temperatures but liquid at the rectal temperature and will therefore melt in the rectum to release the drug. Such materials include cocoa butter, glycerinated gelatin, Hated vegetable oils, mixtures of polyethylene glycols of various molecular weights and fatty acid esters of polyethylene glycol.

For topical use, creams, ointments, jellies, solutions or suspensions, etc., containing a macrolide can be used. As used herein, topical application can include mouth washes and gargles.

The compounds used in the methods and compositions described herein can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

Preferably X—Ar is (CH₂)₂—Ar, CH₂OAr or OCH₂—Ar

More preferably X—Ar is OCH₂—Ar.

Preferably A is N and B is CH or N.

More preferably A is N and B is CH.

Preferably Ar is phenyl, fluorophenyl or chlorophenyl.

More preferably Ar is phenyl, 4-chlorophenyl or 4-fluorophenyl.

Most preferably Ar is phenyl.

Preferably R¹ is piperidine, pyrrolidine, piperazine, octahydro-pyrrolo[3,4-c]pyrrole, octahydro-pyrrolo[3,4-b]pyrrole, or octahydro-pyrrolo[3,4-c]pyridine,

attached to the “ABCCHCHC” ring via a N atom,

which ring system is optionally substituted by one or more substituents independently selected from ═O, C₁-C₅ alkyl, C₃-C₅ cycloalkyl, OH, C(O)C₁-C₅ alkyl, C(O)C₃-C₅ cycloalkyl, C(O)OC₁-C₅ alkyl, NR⁶R⁷, NR⁸C(O)R⁹, NR⁸C(O)OR⁹, O(C₁-C₅ alkyl) or O(C₃-C₅ cycloalkyl).

More preferably R¹ is piperidine, pyrrolidine, piperazine, octahydro-pyrrolo[3,4-c]pyrrole, octahydro-pyrrolo[3,4-b]pyrrole, or octahydro-pyrrolo[3,4-c]pyridine,

attached to the “ABCDEC” ring via a N atom,

which ring system is optionally substituted by one or more substituents independently selected from OH, OMe, OEt, Me, Et, NH₂, NHMe, NMe₂, NMeC(O)Me and C(O)Me.

Yet more preferably R¹ is piperidine, pyrrolidine, piperazine, octahydro-pyrrolo[3,4-c]pyrrole, octahydro-pyrrolo[3,4-b]pyrrole, or octahydro-pyrrolo[3,4-c]pyridine,

attached to the “ABCDEC” ring via a N atom,

which ring system is optionally substituted by one or more substituents independently selected from NHMe, NMe₂, OH, NH₂, Me and Et.

Most preferably R¹ is selected from one of the following groups:

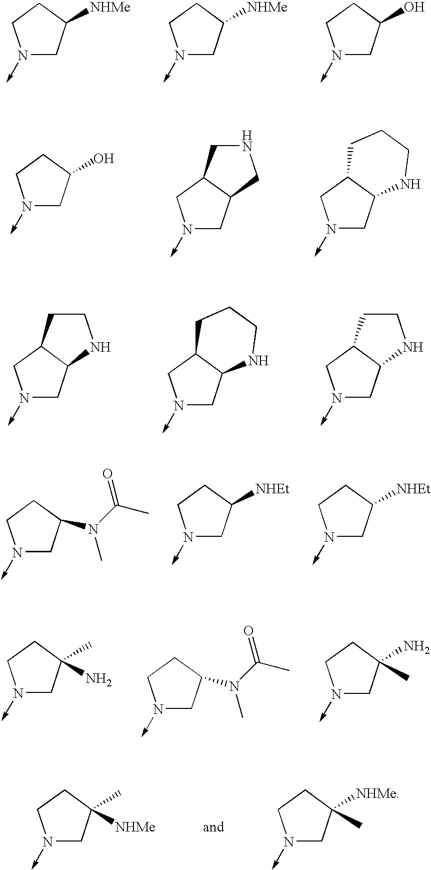

Another preferred aspect of the invention is a group of compounds wherein A, B, X, R¹ and Ar are selected from the values in the compounds of the Examples below, or a pharmaceutically acceptable salt, solvate or prodrug thereof.

Another preferred aspect of the invention is a group of compounds of formula 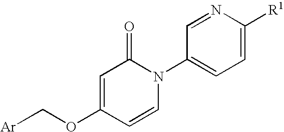
wherein Ar and R¹ are as defined above, preferably wherein Ar is phenyl, or a pharmaceutically acceptable salt, solvate or prodrug thereof.

Another preferred aspect of the invention is a group of compounds of formula 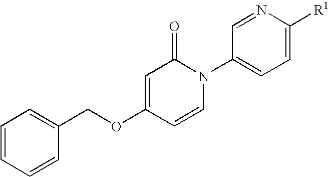
wherein R¹ is selected from 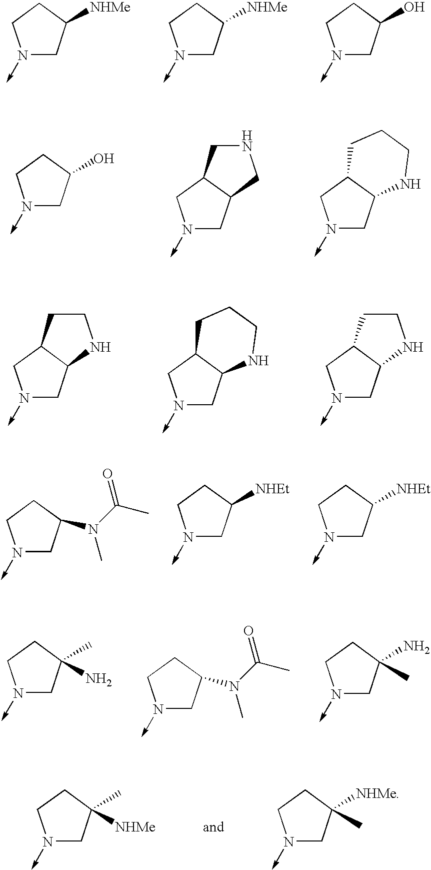
or a pharmaceutically acceptable salt, solvate or prodrug thereof.

Preferably the compounds of the invention are selected from one of the Examples below, or a pharmaceutically acceptable salt, solvate or prodrug thereof.

More preferably the compounds of the invention are selected from the compounds of Examples 17a, 17b, 18a, 18b, 19 or 20, or a pharmaceutically acceptable salt, solvate or prodrug thereof.

Most preferably the compounds of the invention are selected from the compounds of Examples 17a, 18a, 19 or 20, or a pharmaceutically acceptable salt, solvate or prodrug thereof.

Compounds of the invention may be prepared in a variety of ways, including those mentioned in the Examples and Preparations. The routes below illustrate methods of synthesising compounds of formula (I), wherein, unless otherwise described, the substituents have the values mentioned above with regard to formula (I).

The skilled person will appreciate that the compounds of the invention could be made by methods other than those specifically described herein, by the methods described herein and/or adaptation thereof, for example by adaptation of methods known in the art, including the art mentioned earlier herein, such as WO2003068230, WO2005018557 and WO2005103039. Suitable guides to synthesis, functional group interconversions, use of protecting groups, etc., are for example: Comprehensive Organic Transformations” by R C Larock, VCH Publishers Inc. (1989); Advanced Organic Chemistry” by J. March, Wiley Interscience (1985); “Designing Organic Synthesis” by S Warren, Wiley Interscience (1978); “Organic Synthesis—The Disconnection Approach” by S Warren, Wiley Interscience (1982); “Guidebook to Organic Synthesis” by R K Mackie and D M Smith, Longman (1982); “Protective Groups in Organic Synthesis” by T W Greene and P G M Wuts, John Wiley and Sons, Inc. (1999); and “Protecting Groups” by P J, Kocienski, Georg Thieme Verlag (1994); and any updated versions of said standard works.

Compounds of Formula I (Scheme 1) can be prepared by a coupling reaction of a fragment II with a fragment III, where X¹ is a suitable leaving group, for example iodide, bromide or triflate (preferably iodide).

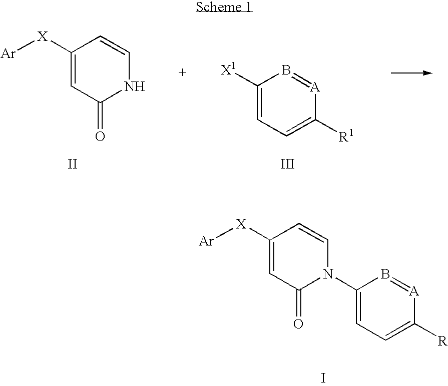

Compounds of Formula II and III are mixed in a suitable solvent such as toluene, DMSO or DMF (preferably DMF), at a temperature between about 50 and about 150° C. (preferably about 80° C.) in the presence of a suitable copper catalyst (preferably CuI) and bidentate ligand (preferably trans-1,2-diaminocyclohexane). This reaction is exemplified in Example 16. Alternatively, where X¹ is a suitable group, for example a boron derivative (preferably B(OH)₂), a coupling reaction can be carried out in a suitable solvent, for example dichloromethane, tetrahydrofuran or acetonitrile (preferably dichloromethane) mediated by a suitable metal salt, for example a copper(II) derivative (preferably Cu(OAc)₂), in the presence of a suitable base, for example pyridine, triethylamine or DBU (preferably pyridine) and a suitable drying agent (e.g. 4 Å molecular sieves) at a temperature between about 25 and about 50° C. (preferably about 25° C.).

Compounds of formula (II) and (III) are available commercially, or can be made by methods disclosed herein, for example in relation to the Examples and Preparations below, or suitable adaptation thereof by methods known in the art.

Compounds of Formula III (Scheme 2) can be suitably prepared by a displacement reaction from compound VI, where X¹ and X² are suitable halogen substituents (preferably bromo), using a suitable amine “R¹H”, a suitable base, for example triethylamine, sodium carbonate or potassium carbonate (preferably potassium carbonate), in a suitable solvent, for example acetonitrile, n-butanol or dimethyl sulfoxide (preferably n-butanol) at a temperature between about 25 and about 150° C. (preferably about 110° C.).

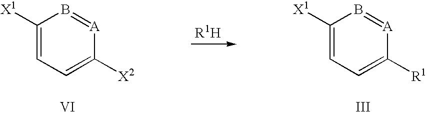

Some of the amines R¹H are available commercially (e.g. see Table below), others are available from the literature or suitable adaptation thereof using methods known to the skilled person.

This reaction is exemplified in Preparation 6.

| Name | Structure | Supplier |
|---|---|---|
| HEXAHYDRO- PYRROLO[3,4-B]PYRROLE-1- CARBOXYLIC ACID TERT-BUTYL ESTER | 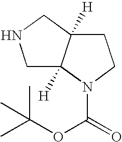 | JW- Pharm- lab |
| (3AS,6AS)-TERT- BUTYL HEXAHYDRO- PYRROLO[3,2-B]PYRROLE-1(2H)- CARBOXYLATE | 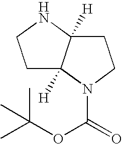 | Mile- stone |
| (3AR,6AS)-TERT- BUTYL HEXAHYDRO- PYRROLO[3,2-B]PYRROLE-1(2H)- CARBOXYLATE | 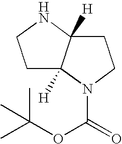 | Mile- stone |
| 1-BOC-OCTAHYDRO- PYRROLO[3,4-B]PYRIDINE | 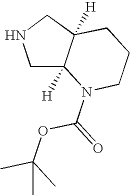 | Tyger |
| TERT-BUTYL (1S,4S)- (−)-2,5-DIAZABI- CYCLO[2.2.1]HEPTANE-2- CARBOXYLATE | 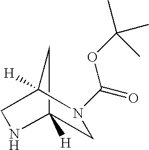 | Aldrich |
| 2,7-DIAZA- SPIRO[4.4]NONANE-2- CARBOXYLIC ACID TERT-BUTYL ESTER | 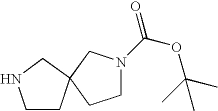 | JW- Pharm- lab |
| TERT-BUTYL 1,7- DIAZASPIRO[4.4]NONANE-1- CARBOXYLATE | 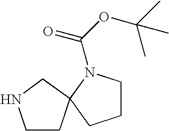 | Mile- stone |
| TERT-BUTYL 1,4- DIAZEPANE-1- CARBOXYLATE | 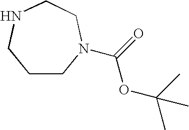 | Aldrich |
| 2,7-DIAZASPIRO[4.5]DECANE-7- CARBOXYLIC ACID T-BUTYL ESTER | 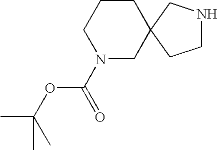 | Syntech |
| 1-(TERT-BUTOXY- CARBONYL)-4- PHENYLPROLINE | 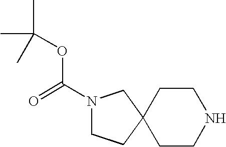 | JW- Pharm- lab |
| 8-BOC-3,8-DIAZA- BICYCLO[3.2.1]OCTANE | 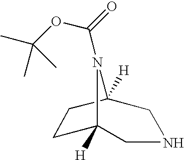 | JW- Pharm- lab |
| TERT-BUTYL 2,7- DIAZASPIRO[4.5]DECANE-2- CARBOXYLATE | 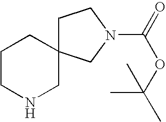 | Mile- stone |

Compounds of Formula VII (Scheme 3) can be prepared by a coupling reaction of a fragment II with a fragment VI, where X¹ can act a suitable leaving group for such a coupling reaction, for example triflate, iodo or bromo (preferably iodo), and X² is a also a suitable leaving group for subsequent displacement by the amine R¹H, for example chloro or fluoro (preferably fluoro).

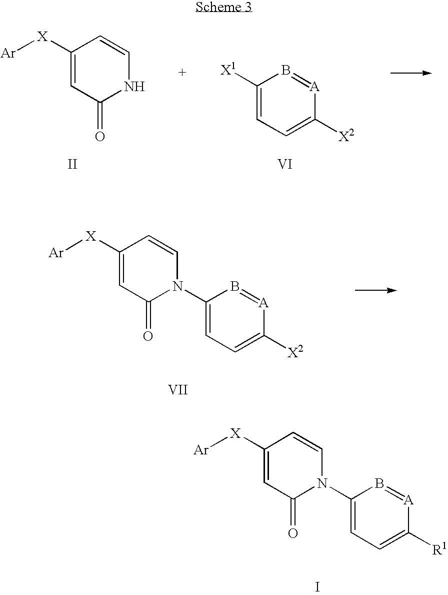

The reactants II and VI are mixed in a suitable solvent such as DMSO or N,N-dimethylformamide (preferably N,N-dimethylformamide), at a temperature between about 25 and about 150° C. (preferably about 80° C.) in the presence of a suitable copper catalyst (preferably CuI) and ligand (preferably trans-1,2-diaminocyclohexane). This reaction is exemplified in Preparation 2 Method B. Alternatively, where X¹ is a suitable group, for example a boron derivative (preferably B(OH)₂), a coupling reaction can be carried out in a suitable solvent, for example dichloromethane, tetrahydrofuran or acetonitrile (preferably dichloromethane) mediated by a suitable metal salt (preferably Cu(OAc)₂) in the presence of a suitable base, for example pyridine or triethylamine (preferably pyridine) and a suitable drying agent (preferably 4 Å molecular sieves) at a temperature between about 25 and about 100° C. (preferably about 25° C.). Compounds of Formula I can be prepared from fragments of Formula VII by treating with a suitable amine R¹H using a suitable solvent, for example acetonitrile, dimethyl sulfoxide or N,N-dimethylformamide (preferably dimethyl sulfoxide), using a suitable base, for example potassium carbonate, cesium carbonate or triethylamine (preferably potassium carbonate) at a temperature between about 25 and about 150° C. (preferably about 110° C.). The reaction is exemplified in Examples 1-7.

Compounds of Formula VIII (Scheme 4) can be transformed to versatile intermediates of Formulae IX and X, where R100 is a suitable substituent, for example phenyl, 4-bromophenyl or trifluoromethyl.

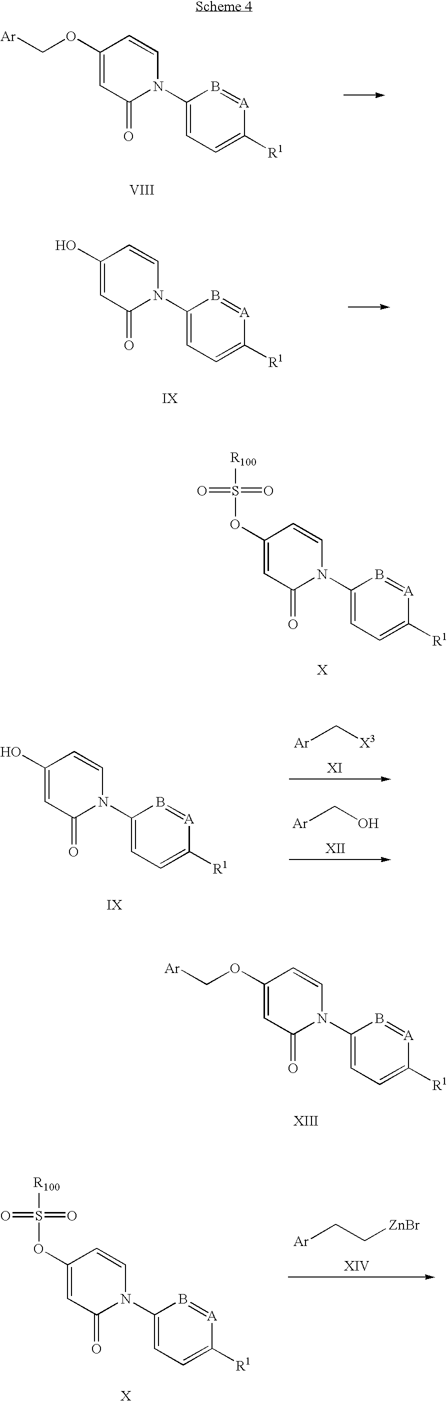

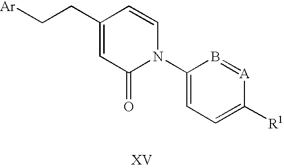

Treatment of compounds of Formula VIII in a suitable solvent, for example ethanol, methanol or acetonitrile (preferably ethanol), with a suitable hydrogen source, for example dihydrotoluene, H₂ gas or ammonium formate (preferably dihydrotoluene) and a suitable metal catalyst (preferably palladium hydroxide on carbon), at a temperature between about 25 and about 100° C., (preferably about 60° C.) gives compounds of Formula IX. The reaction is exemplified in Preparation 4. Compounds of Formula IX can be converted to compounds of Formula X using a suitable source of R100-sulfonyl, e.g. trifluoromethane sulfonic anhydride, in a suitable solvent, for example dichloromethane, ethyl acetate or tetrahydrofuran (preferably dichloromethane), using a suitable base, for example triethylamine or pyridine, at a temperature between about −40° C. and about 25° C. (preferably about 25° C.). Compounds of Formula IX can be converted to compounds of Formula XIII using a range of conditions. For example, treatment of fragments IX with a suitable alkylating agent ArCH₂X³ (XI), where X³ is a suitable leaving group, for example a halide, mesylate, tosylate or triflate (preferably bromide), in a suitable solvent, for example acetonitrile or N,N-dimethylformamide (preferably acetonitrile) with a suitable base, for example cesium carbonate or potassium carbonate (preferably cesium carbonate), at a temperature between about 25 and about 150° C. (preferably about 80° C.). This reaction is exemplified in Example 10. Alternatively fragments of Formula IX can be alkylated via a Mitsonobu reaction using a suitable alcohol of Formula XII using suitable reagents, for example di-isopropyldiazodicarboxylate or diethyldiazodicarboxylate (preferably di-isopropyldiazodicarboxylate) and triphenylphosphine or tributylphosphine (preferably triphenylphosphine), in a suitable solvent, for example tetrahydrofuran or acetonitrile (preferably tetrahydrofuran) at a temperature between about 0° C. and about 100° C. (preferably about 25° C.). Compounds of Formula X can also be transformed to compounds of Formula XV using a variety of conditions. For example, treatment of compounds of Formula X with a suitable organozinc reagent of Formula XIV in a suitable solvent, for example tetrahydrofuran or 2-methyl tetrahydrofuran (preferably tetrahydrofuran), optionally with a suitable co-solvent, for example N-methylpyrrolidinone, with a suitable metal catalyst, (preferably bis(tri-t-butylphosphine)palladium) at a temperature between about 25° C. and about 100° C., (preferably about 50° C.) gives compounds of Formula XV.

Some compounds of formula (I) may be converted into other compounds of formula (I) by known functional group interconversions.

All of the above reactions and the preparations of novel starting materials used in the preceding methods are conventional and appropriate reagents and reaction conditions for their performance or preparation as well as procedures for isolating the desired products will be well-known to those skilled in the art with reference to literature precedents and the Examples and Preparations herein described. Furthermore, it will be clear to the skilled person that the compounds of formula (I) may be synthesised either directly from, or by analogy to, the schemes, literature precedents and the Examples and Preparations herein described, as well as the common general knowledge,

### EXAMPLES OF PHARMACEUTICAL COMPOSITIONS

### Example 1

### Parenteral Composition

To prepare a parenteral pharmaceutical composition suitable for administration by injection, 100 mg of a water-soluble salt of a compound of Formula I is dissolved in DMSO and then mixed with 10 mL of 0.9% sterile saline. The mixture is incorporated into a dosage unit form suitable for administration by injection.

### Example 2

### Oral Composition

To prepare a pharmaceutical composition for oral delivery, 100 mg of a compound of Formula I is mixed with 750 mg of lactose. The mixture is incorporated into an oral dosage unit for, such as a hard gelatin capsule, which is suitable for oral administration.

Biological Testing

hERG Binding Assay

Membrane homogenates of HEK-293S (Cell line #15-08) cells expressing the HERG product supplied by (PGRD) Sandwich Laboratories were prepared as follows. Cell pellets were thawed at room temperature and kept on ice. Buffer (50 mM Tris.HCl, 1 mM MgCl2, 10 mM KCl, pH 7.4, at 4° C.) was added to each cell pellet (10 ml of buffer per 10 g of packed cell pellet) and the mixture homogenised using an Omni LabTek homogeniser (20,000 rpm for 30 seconds). The homogenate was centrifuged at 48,000 g for 20 minutes between 3 and 5° C. in a Sorvall Evolution RC centrifuge and the supernatant discarded. The pellet was resuspended, homogenised (20,000 rpm for 10 seconds), and centrifuged as before. The resultant supernatant was discarded and the final pellet resuspended (100 ml of the above buffer per 10 g of packed cell pellet), homogenised (20,000 rpm for 10 seconds), dispensed in to tubes in 1, 2 and 5 ml aliquots and stored between −75° C. and −85° C. until use. Protein concentration was determined using a Coomassie Blue kit as per manufacturers instructions (Sigma 610A & 610-11).

The Cy3B ligand was stored in 100% DMSO and diluted to 6 nM in assay buffer (50 mM Tris.HCl, 1 mM MgCl2, 10 mM KCl, 0.05% Pluronic F127, pH 7.4 at 4° C.) on the day of the experiment. Test samples and controls were diluted in 6% DMSO, 0.05% Pluronic F127. Cell membranes were removed from the −80° C. freezer and placed on ice after defrosting. When required the defrosted membranes were homogenised using a polytronic device for no more than 10 seconds, they were then diluted in the above assay buffer to produce a working solution of 0.3 mg/ml. The assay was compiled by adding 10 mL of test compound or control solution, 10 mL of the Cy3B ligand and 10 mL of cell membranes to a black 384-well plate (Matrix, Cat No. 4318). The plates were mixed and then incubated for a minimum of 2 hours prior to reading on a Tecan Ultra (Excitation 530 nm, Emission 590 nm). All IC₅₀ and Kᵢ data were generated using Pfizer proprietary software.

The practice of the instant invention for treating obesity or related eating disorders (including promoting weight loss or reducing weight gain) can be evidenced by activity in at least one of the protocols described hereinbelow.

Food Intake

The following screen is used to evaluate the efficacy of test compounds for inhibiting food intake in Sprague-Dawley rats after an overnight fast.

Male Sprague-Dawley rats are obtained from Charles River Laboratories, Inc. (Wilmington, Mass.). The rats are individually housed and fed powdered chow. They are maintained on a 12 hour light/dark cycle and received food and water ad libitum. The animals are acclimated to the vivarium for a period of one week before testing is conducted. Testing is completed during the light portion of the cycle.

To conduct the food intake efficacy screen, rats are transferred to individual test cages without food the afternoon prior to testing, and the rats are fasted overnight. After the overnight fast, rats are dosed the following morning with vehicle or test compounds. A known antagonist is dosed (3 mg/kg) as a positive control, and a control group receives vehicle alone (no compound). The test compounds are dosed at ranges between 0.1 and 100 mg/kg depending upon the compound. The standard vehicle is 0.5% (w/v) methylcellulose in water and the standard route of administration is oral. However, different vehicles and routes of administration are used to accommodate various compounds when required. Food is provided to the rats 30 minutes after dosing and the Oxymax automated food intake system (Columbus Instruments, Columbus, Ohio) is started. Individual rat food intake is recorded continuously at 10-minute intervals for a period of two hours. When required, food intake is recorded manually using an electronic scale; food is weighed every 30 minutes after food is provided up to four hours after food is provided. Compound efficacy is determined by comparing the food intake pattern of compound-treated rats to vehicle and the standard positive control.

Oxygen Consumption

Whole body oxygen consumption is measured using an indirect calorimeter (Oxymax from Columbus Instruments, Columbus, Ohio) in male Sprague Dawley rats (if another rat strain or female rats is used, it will be specified). Rats (300-380 g body weight) are placed in the calorimeter chambers and the chambers are placed in activity monitors. These studies are done during the light cycle. Prior to the measurement of oxygen consumption, the rats are fed standard chow ad libitum. During the measurement of oxygen consumption, food is not available. Basal pre-dose oxygen consumption and ambulatory activity are measured every 10 minutes for 2.5 to 3 hours. At the end of the basal pre-dosing period, the chambers are opened and the animals are administered a single dose of compound (the usual dose range is 0.001 to 10 mg/kg) by oral gavage (or other route of administration as specified, i.e. s.c., i.p., i.v.). Drugs are prepared in methylcellulose, water or other specified vehicle (examples include PEG400, 30% beta-cyclo dextran and propylene glycol). Oxygen consumption and ambulatory activity are measured every 10 minutes for an additional 1-6 hours post-dosing.

The Oxymax calorimeter software calculates the oxygen consumption (ml/kg/h) based on the flow rate of air through the chambers and difference in oxygen content at inlet and output ports. The activity monitors have 15 infrared light beams spaced one inch apart on each axis, ambulatory activity is recorded when two consecutive beams are broken and the results are recorded as counts.

Resting oxygen consumption, during pre- and post-dosing, is calculated by averaging the 10-min O₂ consumption values, excluding periods of high ambulatory activity (ambulatory activity count >100) and excluding the first 5 values of the pre-dose period and the first value from the post-dose period. Change in oxygen consumption is reported as percent and is calculated by dividing the post-dosing resting oxygen consumption by the pre-dose oxygen consumption*100. Experiments will typically be done with n=4-6 rats and results reported are mean +/−SEM.

An increase in oxygen consumption of >10% is considered a positive result. Historically, vehicle-treated rats have no change in oxygen consumption from pre-dose basal.

MCHR Stable Cell Lines

CHO cell lines stably expressing the human MCHR1 (Euroscreen, Brussels, Belgium) were maintained in DMEM containing Glutamax (Invitrogen, Carlsbad, Calif.), 10% FBS, 400 ug/ml Geneticin.

Cell Membrane Preparation. CHO frozen pellets (from two 10-layer factories) containing human MCHR1 receptors where thawed on ice and homogenized in buffer A containing: 50 mM Tris (pH 7.4), 0.32 M Sucrose, 1 mM EDTA, 1 mM EGTA, 1 mM sodium bicarbonate, 10 μg/mg Benzamadine, 10 μg/ml Bacitracin, 5 μg/ml Aprotinin, 5 μg/ml Leupeptin. The homogenate was centrifuged at 4° C. at 6000×g for 10 min. The supernatant was removed and placed on ice. The pellet was re-suspended in buffer A and re-centrifuged at 4° C. at 6000×g for an additional 10 min. Supernatants were pooled and spun at 48,000×g for 30 minutes at 4° C. The final pellet was resuspended in 5 mls Buffer A. The final membrane protein was determined with a BCA kit (Pierce, Rockford, Ill.). Membranes were stored at −70° C. at a protein concentration of approximately 5 mg/ml.

Cyclic AMP assays. CHO cells stably expressing the human MCHR1 receptors were collected and resuspended at a density of 4.0×10⁶ cells/ml in F12 (Hams) media and seeded (20,000 cells per well) into 384-well, solid white Greiner assay plates. For antagonists studies, 11-point 3-fold dose-response curves were constructed (top final assay concentration 30 uM) in PBS with 0.01% pluronic and 0.3% DMSO and added to the cells along with 25 uM forskolin and 0.2 nM MCH (final assay concentration). Cells were incubated at 37° C./5% CO₂ for 90 minutes and cAMP accumulation subsequently determined by DiscoveRx Hithunter c-AMP II Assay kit (GE Healthcare, Buckinghamshire, UK) in accordance with manufactures instructions. Luminescence was measured on a LEADseaker imaging system (GE Healthcare, Buckinghamshire, UK). IC50 values were calculated from dose-response data fit using an in-house computer graphics and statistics program. The functional Ki was calculated using the Cheng-Prusoff equation Ki=IC50/1+[MCH]/EC50(MCH) where [MCH]=0.2 nM and EC50 (MCH)=0.1 nM

Biological Data

| | MCHR-1 functional |
|---|---|
| Example Number | Ki (nM) |
| 1 | 47.7 |
| 2 | 89.9 |
| 3 | 10.1 |
| 4 | 1950 |
| 5 | 111 |
| 6 | 163 |
| 7 | 87.1 |
| 8 | 8.67 |
| 10 | 42.8 |
| 11 | 97.2 |
| 12 | 9.98 |
| 13 | 4.88 |
| 14 | nd |
| 15 | 3.62 |
| 16 | 133 |
| 17b | 38.1 |
| 17a | 15.3 |
| 18a | 8.86 |
| 18b | 18.3 |
| 19 | 6.78 |
| 20 | 15.2 |
| 21 | nd |
| 21a | nd |
| 21b | nd |
| 22a | nd |
| 22b | nd |
| 23a | 15.7 |
| 23b | 31.4 |
| 24 | 120 |
| 25 | 80.9 |
| 25a | 71.1 |
| 25b | 69.6 |
| 26 | 6.64 |
| 27 | 71.4 |
| 28 | nd |
| 29 | 23.7 |
| 30 | 2470 |
| 32 | 18.3 |
| 33 | 19.4 |
| 34 | 54.3 |
| 35 | 232 |
| 36 | 75.5 |
| 37 | 42.7 |
| 38a | 49.9 |
| 38b | 7.16 |
| 39 | 42 |
| 40 | 34.1 |
| 41a | 19.9 |
| 41b | 19.2 |
| 42a | 24.4 |
| 42b | 127 |
| 43 | 364 |
| 44 | 113 |
| 45 | 270 |
| 46 | 212 |
| 47 | 57.8 |
| 48 | 705 |
| 49 | 15.9 |
| 50 | 271 |
| 51 | 258 |
| 52 | 5.92 |
| 53a | 47.6 |
| 53b | 10 |
| 54 | 5.91 |
| 55 | 3.66 |
| 56 | 3.69 |
| 57 | 3.66 |
| 58 | 28.8 |
| 59 | 68.3 |
| 60 | 2.94 |
| 61 | 18.7 |
| 62a | 16.8 |
| 62b | 11.4 |
| 63 | 12.1 |
| 64 | 86.7 |
| 65 | 40.2 |
| 66 | 85.5 |
| 67 | 188 |
| 68 | 119 |
| 69 | 124 |
| 70 | 102 |
| 71 | 626 |
| 72 | 74.6 |
| 73 | 517 |
| 74 | 7.37 |
| 75 | 24 |
| 76 | 3840 |
| 77 | 141 |
| 78 | 155 |
| 79 | 416 |
| 80 | 133 |
| 81 | 1170 |
| 82a | 28.5 |
| 82b | 36.7 |
| 83 | 851 |
| 84 | 19.3 |
| 85 | 15.7 |
| 86 | 7.75 |
| 87 | 191 |
| 88 | 1140 |
| 89a | 3.45 |
| 89b | 5.06 |
| 90 | 96.7 |
| 91 | 396 |
| 92 | 711 |
| 93 | 1090 |
| 94 | 116 |
| 95 | 147 |
| 96 | 195 |
| 97 | 49.5 |
| 98 | 8.23 |
| 99 | 5.31 |
| 100a | 2.67 |
| 100b | 9.82 |
| 101 | 25.8 |
| 102 | 34.8 |
| 103 | 25.8 |
| 104 | 36.6 |
| 105 | 6.34 |
| 106 | 65.8 |
| 107 | 5.43 |
| 108 | 16.8 |
| 109 | 22.7 |
| 110 | 41.6 |
| 111 | 161 |
| 112 | 184 |
| 113 | 50.4 |
| 114 | 47.9 |
| 115 | 100 |
| 116 | 26.9 |
| 117 | 1690 |
| 118 | 11.5 |
| 119 | nd |
| 120 | nd |
| 121 | 37.1 |
| 122 | 110 |
| 123a | 12.7 |
| 123b | nd |
| 124 | nd |
| 125 | 4.46 |
| 126 | 9.92 |
| 127 | 417 |
| 128 | 659 |
| 129 | 6.9 |
| 130 | 9.31 |
| 131 | 9.15 |
| 132 | 7.95 |
| 133 | 3.23 |
| 134 | 2.9 |
| 135 | 4.86 |
| 136 | 4.48 |
| 137 | 234 |
| 138 | 26.8 |
| 139a | 37.8 |
| 139b | 172 |
| 140 | 94.7 |
| 141 | 100 |
| 142 | 105 |
| 143 | 5.68 |
| 144 | 69.9 |
| 145 | 98.5 |
| 146 | 60.1 |
| 147 | 26.2 |
| 148 | 125 |
| 149 | 3.46 |
| 150 | 3.47 |
| 151 | 6.04 |
| 152 | 2.31 |
| 153 | 2.02 |
| 154 | 3.99 |
| 155 | 3800 |
| 156 | 230 |
| 157 | 3160 |
| 158 | 49.8 |
| 159 | 201 |
| 160 | 26.8 |
| 161 | 33.3 |
| 162 | 30.9 |
| 163 | 83.8 |
| 164 | 66.8 |
| 165 | 29.2 |
| 166 | 1210 |
| 167 | 749 |
| 168 | 539 |
| 169 | 19.4 |
| 170 | 29.1 |
| 171 | 38.1 |
| 172 | 11 |
| 173 | 13.1 |
| 174 | 26.2 |
| 175 | 58.5 |
| 176 | 29.9 |
| 177 | 19.3 |
| 178 | 19.2 |
| 179 | 18.9 |
| 180 | 22.2 |
| 181 | 10.9 |
| 182 | 42.9 |
| 183 | 45.9 |
| 184 | 42 |
| 185 | 42.6 |
| 186 | 29.8 |
| 187 | 24.1 |
| 188 | 218 |
| 189 | 72 |
| 190 | 117 |
| 191a | 292 |
| 191b | 24.7 |
| 192 | 144 |
| 193 | 196 |
| 194 | 46.6 |
| 195 | 31.3 |
| 196 | 605 |
| 197 | 7.7 |
| 198 | 10.6 |
| 199 | 110 |
| 200 | 57 |
| 201a | 348 |
| 201b | 112 |
| 202 | 46.3 |
| 203a | 431 |
| 203b | 6.48 |
| 204 | 330 |
| 205 | 498 |
| 206 | 250 |
| 207 | 6.51 |
| 208 | 2.72 |
| 209 | 10.6 |
| 210 | 4.56 |
| 211 | 19.4 |
| 212 | 14.3 |
| 213 | 168 |
| 214 | 106 |
| 215 | 161 |
| 216 | 78 |
| 217 | 11 |
| 218 | 14.9 |
| 219 | 8.45 |
| 220 | 513 |
| 221 | 126 |
| 222 | 680 |
| 223 | 197 |
| 224 | 16.6 |
| 225 | 8.03 |
| 226 | 2480 |
| 227 | 20.8 |
| 228 | 6.18 |
| 229 | 2480 |
| 230 | 69.8 |
| 231 | 752 |
| 232 | 238 |
| 233 | 17.6 |
| 234 | 3.47 |
| 235 | 10.9 |
| 236 | 34.7 |
| 237 | 101 |
| 238 | 76.1 |
| 239 | 41.2 |
| 240 | 77.5 |

### COMPOUND EXAMPLES AND PREPARATIONS

The invention is illustrated by the following non-limiting examples in which the following abbreviations and definitions are used.
APCI atmospheric pressure chemical ionisation
Ac acetyl
Arbocel® filter agent
BINAP 2,2′-bis(diphenylphosphino)-1′,1-binaphthyl
br broad
Boc tert-butoxycarbonyl
Bu butyl
CDCl₃ chloroform-d1
CD₃OD methanol-d4
δ chemical shift
d doublet
dd double doublet
DCM dichloromethane
DIEA N,N-diisopropylethylamine
DMF N,N-dimethylformamide
DMSO dimethylsulfoxide
eq (molar) equivalents
ESI electrospray ionisation
Et ethyl
h hours
HATU O-(7-azabenzotriazole-1-yl)-N,N,N′,N′-tetramethyluronium hexafluorophosphate
HBTU O-(1H-Benzotriazol-1-yl)-N,N,N′,N′-tetramethyluronium hexafluorophosphate
HPLC high performance liquid chromatography
HRMS high resolution mass spectrum
IPA isopropylalcohol

LRMS low resolution mass spectrometry
m multiplet
Me methyl
MS mass spectrum
min minutes
MTBE methyl tert butyl ether
NMP N-methylpyrrolidone
NMR nuclear magnetic resonance
Ph phenyl
Pr n-propyl
Psi pounds per square inch
pTSA p-Toluenesulfonic acid
q quartet
RM reaction mixture
r.t. room temperature
s singlet
sat saturated
SM starting material
soln solution
t triplet
TBDMS Tert butyldimethylsilyl
td triplet of doublets
Tf trifluoromethanesulfonyl
TFA trifluoroacetic acid
THF tetrahydrofuran
TIPS triisopropylsilyl
TLC/t.l.c thin layer chromatography

¹H Nuclear magnetic resonance (NMR) spectra were in all cases consistent with the proposed structures. Characteristic chemical shifts (δ) are given in parts-per-million [symbol] _{H} downfield from tetramethylsilane using conventional abbreviations for designation of major peaks: e.g. s, singlet; d, doublet; t, triplet; q, quartet; m, multiplet; br, broad.

Where compounds were analysed by LCMS several methods were used shown below—

6 Minute LC-MS Gradient and Instrument Conditions

Acid Run:

A: 0.1% formic acid in water

B: 0.1% formic acid in acetonitrile

Column: C18 phase Phenomenex Gemini 50×4.6 mm with 5 micron particle size

Gradient: 95-5% A over 3 min, 1 min hold, 1 ml/min

UV: 210 nm-450 nm DAD

Temperature: 50 C

Basic Run:

A: 0.1% ammonium hydroxide in water

B: 0.1% ammonium hydroxide in acetonitrile

Column: C18 phase Fortis 50×4.6 mm with 5 micron particle size

Gradient: 95-5% A over 3 min, 1 min hold, 1 ml/min

UV: 210 nm-450 nm DAD

Temperature: 50 C

2 Minute LC-MS Gradient and Instrument Conditions

Acid Run:

A: 0.1% formic acid in water

B: 0.1% formic acid in acetonitrile

Column: C18 phase Fortis Pace 20×2.1 mm with 3 micron particle size

Gradient: 70-2% A over 1.8 min, 0.2 min hold, 1.8 ml/min

UV: 210 nm-450 nm DAD

Temperature: 75 C

Method A: 4.7 Minute LC-MS Gradient and Instrument Conditions

A: 3.75% trifluoroacetic acid in water

B: 1.88% trifluoroacetic acid in acetonitrile

Column: Ymc ODS-AQ 2×50 mm with 5 micron particle size

Gradient: 0 min: 1% B; 0.6 mins: 5% B; 4 min: 100% B; 4.3 mins: 1% B

Flow: 0.8 ml/min

UV: 210 nm DAD

Temperature: 50 C

Method B: 4.7 Minute LC-MS Gradient and Instrument Conditions

A: 3.75% trifluoroacetic acid in water

B: 1.88% trifluoroacetic acid in acetonitrile

Column: Ymc ODS-AQ 2×50 mm with 5 micron particle size

Gradient: 0 min: 10% B; 0.5 mins: 10% B; 4 min: 100% B; 4.3 mins: 10% B

Flow: 0.8 ml/min

UV: 210 nm DAD

Temperature: 50 C

Method C: 4.7 Minute LC-MS Gradient and Instrument Conditions

A: 0.5% ammonium hydroxide in water

B: acetonitrile

Column: Welch XB-C18 2.1×50 mm with 5 micron particle size

Gradient: 0 min: 5% B; 0.5 mins: 5% B; 3.4 min: 100% B; 4.2 mins: 100% B; 4.2 min 5% B

Flow: 0.8 ml/min

UV: 210 nm DAD

Temperature: 50 C

Where compounds are purified by HPLC, there are two methods used, shown below.

| | Method D | Method E |
|---|---|---|
| Column | Sunfire C18 4.6 × 50 mm id | Xterra 4.6 × 50 mm id |
| | column | column |
| Temperature | Ambient | Ambient |
| Mobile Phase A | 0.05% formic acid in | 0.05% ammonia in |
| | water | water |
| Mobile Phase B | 0.05% formic acid in | 0.05% ammonia in |
| | acetonitrile | acetonitrile |
| Gradient - Initial | 5% B | 5% B |
| Time 0 mins | 5% B | 5% B |
| Time 3 mins | 98% B | 98% B |
| Time 4 mins | 98% B | 98% B |
| Time 4.1 mins | 5% B | 5% B |
| Time 5 mins | 5% B | 5% B |
| Flow rate | 1.5 ml/min | 1.5 ml/min |
| Injection volume | 5 ul | 5 ul |

### Example 1

### 6′-(4-Acetylpiperazin-1-yl)-4-(benzyloxy)-2H-1,3′-bipyridin-2-one

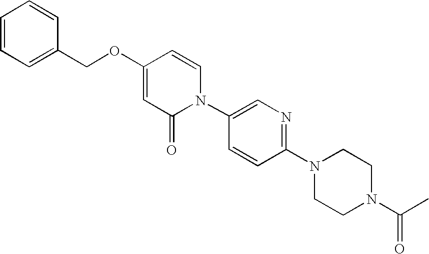

4-(Benzyloxy)-6′-fluoro-2H-1,3′-bipyridin-2-one from Preparation 2 (100 mg, 0.337 mmol), potassium carbonate (140 mg, 1.01 mmol) and N-acetyl piperazine (48 mg, 0.371 mmol) were suspended in N,N-dimethylformamide (1.00 ml) and warmed to 100° C., for 16 hours. The reaction mixture was then cooled to room temperature and then the solvents were evaporated in vacuo. The residue was then triturated with dichloromethane (20 ml) and the solid was removed by filtration. The filtrate was then evaporated to give a brown residue (78 mg). Purification by column chromatography eluting with dichloromethane: methanol: aqueous ammonia (90:10:1) gave the title compound (30 mg, 22%) as an off-white solid. ¹H NMR (400 MHz, CD₃OD) δ ppm 2.15 (d, 2H), 2.17 (s, 3H), 3.58-3.70 (m, 6H), 5.18 (s, 2H), 6.09 (s, 1H), 6.24 (dd, 1H), 6.94 (d, 1H), 7.32-7.45 (m, 5H), 7.50 (d, 1H), 7.58 (dd, 1H), 8.10 (s, 1H). LRMS m/z (APCI) 405 [MH+].

### Example 2

### 4-(Benzyloxy)-6′-piperazin-1-yl-2H-1,3′-bipyridin-2-one

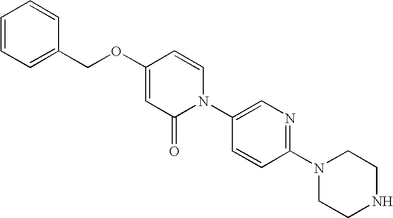

4-(Benzyloxy)-6′-fluoro-2H-1,3′-bipyridin-2-one from Preparation 2 (100 mg, 0.337 mmol), potassium carbonate (140 mg, 1.01 mmol) and piperazine (32 mg, 0.371 mmol) were suspended in N,N-dimethylformamide (1.00 ml) and warmed to 100° C., for 16 hours. The reaction mixture was then cooled to room temperature and then the solvents were evaporated in vacuo. The residue was then triturated with dichloromethane (20 ml) and the solid was removed by filtration. The filtrate was concentrated to give a brown residue (78 mg). Purification by column chromatography eluting with dichloromethane:methanol:aqueous ammonia (90:10:1) gave an off-white solid. This solid was triturated with diethyl ether (5 ml) and then the solid was filtered off and air dried to give the title compound (9 mg, 7%) as a white solid. ¹H NMR (400 MHz, CDCl₃) δ ppm 2.98 (t, 4H), 3.58 (t, 4H), 5.02 (s, 2H), 6.01-6.03 (m, 2H), 6.65 (d, 1H), 7.18-7.20 (m, 1H), 7.31-7.43 (m, 5H), 7.54 (dd, 1H), 8.09 (s, 1H). LRMS m/z (APCI) 363 [MH+].

### Example 3

### 4-(Benzyloxy)-6′-[(3aR,6aS)-5-methylhexahydropyrrolo[3,4-c]pyrrol-2(1H)-yl]-2H-1,3′-bipyridin-2-one

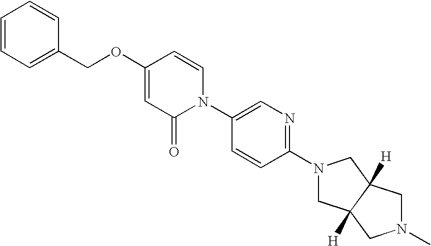

4-(Benzyloxy)-6′-fluoro-2H-1,3′-bipyridin-2-one from Preparation 2 (100 mg, 0.337 mmol), potassium carbonate (140 mg, 1.01 mmol) and 2-methyl-octahydro-pyrrolo[3,4-c]pyrrole (74 mg, 0.371 mmol; ChemStep) were suspended in dimethyl sulfoxide (1.00 ml) and warmed to 100° C. for 16 hours. Warming was ceased and the reaction mixture was diluted with ethyl acetate (30 ml). The reaction mixture was then washed with water (2×20 ml). The organic fraction was dried over anhydrous magnesium sulfate, filtered and then evaporated in vacuo to give a yellow oil. The oil was purified by column chromatography eluting with dichloromethane: methanol: aqueous ammonia (90:10:1) to afford the title compound (50 mg, 37%) as a white solid. ¹H NMR (400 MHz, CDCl₃) δ ppm 2.34 (s, 3H), 2.42 (dd, 2H), 2.77 (t, 2H), 2.98-3.03 (m, 2H), 3.42 (dd, 2H), 3.64 (t, 2H), 5.01 (s, 2H), 6.01-6.02 (m, 2H), 6.42 (d, 1H), 7.18 (d, 1H), 7.32-7.42 (m, 5H), 7.46 (dd, 1H), 8.02 (s, 1H). LRMS m/z (APCI) 403 [MH+]

### Example 4

### tert-Butyl (3aR,6aS)-5-[4-(benzyloxy)-2-oxo-2H-1,3′-bipyridin-6′-yl]hexahydropyrrolo[3,4-c]pyrrole-2(1H)-carboxylate

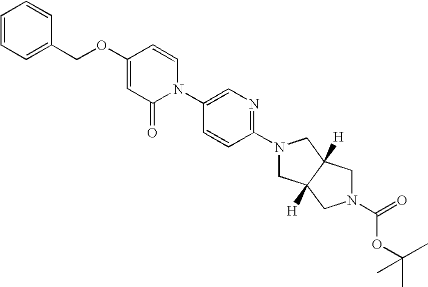

4-(Benzyloxy)-6′-fluoro-2H-1,3′-bipyridin-2-one from Preparation 2 (150 mg, 0.506 mmol), potassium carbonate (210 mg, 1.52 mmol) and hexahydro-pyrrolo[3,4-c]pyrrole-2-carboxylic acid tert-butyl ester (107 mg, 0.506 mmol; Chembasics) were suspended in dimethyl sulfoxide (1.00 ml) and warmed to 100° C. for 16 hours. Heating was ceased and the reaction was cooled. The reaction mixture was then diluted with water (20 ml). An off-white solid precipitated and was filtered and dried under air, then placed in the vacuum oven at 55° C. for 1 hour. The precipitate was purified by column chromatography eluting with dichloromethane: methanol: aqueous ammonia 95:5:0.5 to afford the title compound (111 mg, 45%) as a white solid. ¹H NMR (400 MHz, CDCl₃) δ ppm 1.43 (s, 9H), 2.99-3.04 (m, 2H), 3.22-3.78 (m, 8H), 5.02 (s, 2H), 6.01-6.04 (m, 2H), 6.41 (d, 1H), 7.19 (d, 1H), 7.34-7.42 (m, 5H), 7.50-7.55 (m, 1H), 8.03 (s, 1H). LCMS m/z (APCI) 489 [MH+].

### Example 5

### 4-(Benzyloxy)-6′-[4-(dimethylamino)piperidin-1-yl]-2H-1,3′-bipyridin-2-one

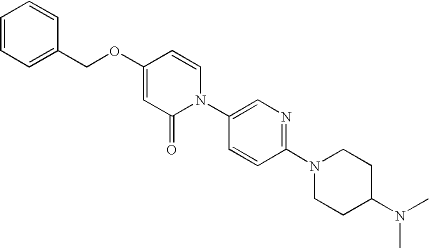

A mixture of 4-(benzyloxy)-6′-chloro-2H-1,3′-bipyridin-2-one from Preparation 3 (50 mg, 0.16 mmol), dimethyl-piperidine-4-yl-amine (62 mg, 0.48 mmol) and cesium fluoride (25 mg, 0.16 mmol) in DMSO (0.5 ml) was heated in microwave oven at 150° C. for 45 minutes. After cooling to room temperature EtOAc (10 ml) and water (10 ml) were added. The organic component was washed with water (2×5 ml), brine (5 ml), dried (MgSO₄), filtered and concentrated. The residue was purified by column chromatography (DCM:MeOH:0.880 NH₃, 95:5:0.5 then 90:10:1). The product-containing fractions were evaporated and the resulting solid was triturated (Et₂O), filtered, washed (pentane) and dried to give the desired product as a white solid (32 mg, 49%). ¹H NMR (400 MHz, CDCl₃,) δ ppm 1.51 (m, 2H), 1.93 (m, 2H), 2.34 (s, 6H), 2.43 (m, 1H), 2.88 (m, 2H), 4.38 (m, 2H), 5.02 (s, 2H), 6.03 (m, 2H), 6.70 (d, 1H), 7.17 (d, 1H), 7.32-7.43 (m, 5H), 7.51 (dd, 1H), 8.06 (d, 1H). LRMS m/z (APCI & ES) 405 [MH⁺].

### Example 6

### 4-(Benzyloxy)-6′-(4-methylpiperazin-1-yl)-2H-1,3′-bipyridin-2-one

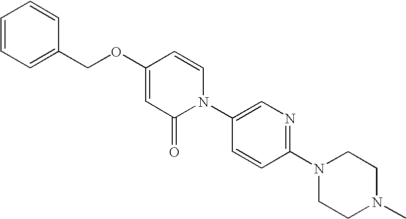

A mixture of the 4-(benzyloxy)-6′-chloro-2H-1,3′-bipyridin-2-one from Preparation 3 (53 mg, 0.16 mmol), 1-methyl piperazine (53 μl, 0.48 mmol) and cesium fluoride (25 mg, 0.16 mmol) in DMSO (0.5 ml) was heated in a microwave oven at 150° C. for 45 minutes. After cooling to room temperature ethyl acetate (10 ml) and water (10 ml) were added. The organic component was separated, washed water (2×5 ml) and then brine (5 ml), dried (MgSO₄), filtered and concentrated. The residue was purified by column chromatography (DCM:MeOH:0.880 NH₃, 95:5:0.5 then 90:10:1). The fractions containing product were concentrated and the resulting solid was triturated (Et₂O), filtered, washed (pentane) and dried to give the desired product as a white solid (25 mg, 42%). ¹H NMR (400 MHz, CDCl₃) δ ppm 2.35 (s, 3H), 2.53 (m, 4H), 3.62 (m, 4H), 5.02 (s, 2H), 6.03 (m, 2H), 6.70 (d, 1H), 7.17 (d, 1H), 7.32-7.44 (m, 5H), 7.53 (dd, 1H), 8.10 (d, 1H). LRMS m/z (APCI & ES) 377 [MH⁺].

### Example 7

### 4-(Benzyloxy)-6′-(4-ethylpiperazin-1-yl)-2H-1,3′-bipyridin-2-one

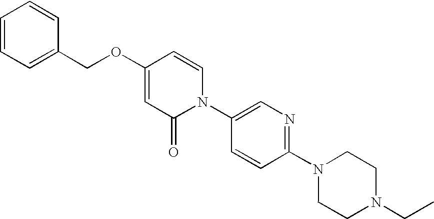

4-(Benzyloxy)-6′-fluoro-2H-1,3′-bipyridin-2-one from Preparation 2 (100 mg, 0.337 mmol), potassium carbonate (140 mg, 1.01 mmol) and N-ethylpiperazine (0.047 mL, 0.371 mmol) were suspended in N,N-dimethylformamide (0.50 ml) and warmed to 100° C. with stirring under N₂ for 16 hours. The reaction mixture was then cooled to room temperature and then the solvents were removed in vacuo. The residue was then triturated with dichloromethane (20 ml) and the insoluble solid was filtered off. The filtrate was then evaporated in vacuo to give a brown residue. This was purified by column chromatography eluting with dichloromethane: methanol: aqueous ammonia (90:10:1) to give an off-white solid (100 mg). The solid was triturated with diethyl ether (20 ml) then filtered to give the title compound (70 mg, 53%) as a white solid. ¹H NMR (400 MHz, CD₃OD) δ ppm 1.17 (t, 3H), 2.50 (q, 2H), 2.60 (t, 4H), 3.62 (t, 4H), 5.18 (s, 2H), 6.05 (s, 1H), 6.23 (dd, 1H), 6.90 (d, 1H), 7.32-7.44 (m, 5H), 7.50 (d, 1H), 7.55 (dd, 1H), 8.03 (s, 1H). LRMS m/z (APCI) 391 [MH+].

### Example 8

### 4-(Benzyloxy)-6′-[(3aR,6aS)-hexahydropyrrolo[3,4-c]pyrrol-2(1H)-yl]-2H-1,3′-bipyridin-2-one dihydrochloride

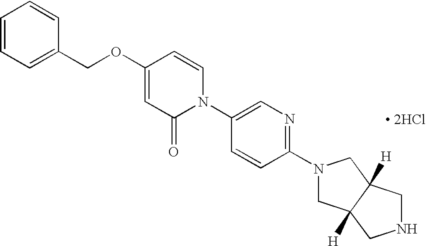

A solution of 4M hydrogen chloride in dioxane (1.09 ml, 4.38 mmol) was added to a solution of tert-butyl (3aR,6aS)-5-[4-(benzyloxy)-2-oxo-2H-1,3′-bipyridin-6′-yl]hexahydropyrrolo[3,4-c]pyrrole-2(1H)-carboxylate from Example 4 (107 mg, 0.22 mmol) in dichloromethane (2 ml) at room temperature. Immediately after addition a white precipitate appeared. The reaction mixture was stirred for 2 hours then the solvents were evaporated in vacuo. The resultant white solid was azeotroped with dichloromethane (2×20 ml) then dried in vacuo to afford the title compound (102 mg, 100%) as a white solid. ¹H NMR (400 MHz, CD₃OD) δ ppm 3.37-3.49 (m, 4H), 3.62-3.70 (m, 2H), 3.79 (dd, 2H), 3.98 (dd, 2H), 5.19 (s, 2H), 6.10 (d, 1H), 6.35 (dd, 1H), 7.22 (d, 1H), 7.33-7.44 (m, 5H), 7.59 (d, 1H), 8.09 (dd, 1H), 8.18 (s, 1H). LRMS m/z (APCI) 389 [MH+].

### Example 9

### 4-[(4-Chlorobenzyl)oxy]-6′-[3-(methylamino)pyrrolidin-1-yl]-2H-1,3′-bipyridin-2-one

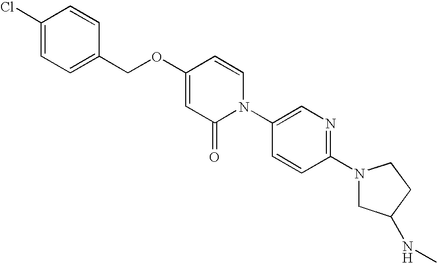

(3R)-1-{4-[(4-Chlorobenzyl)oxy]-2-oxo-2H-1,3′-bipyridin-6′-yl}pyrrolidin-3-yl methanesulfonate from Preparation 5 (20 mg, 0.04 mmol) and 2M methylamine in THF (0.53 ml, 1.1 mmol) were heated at 100° C. in a reacti-vial for 10 days. After cooling to room temperature the reaction mixture was concentrated and purified by preparative HPLC, which gave the desired product as a white solid (3 mg, 18%). ¹H NMR (CD₃OD, 400 MHz) δ ppm 1.93-2.02 (m, 1H), 2.27-2.35 (m, 1H), 2.43 (s, 3H), 3.32-3.38 (m, 1H), 3.41-3.58 (m, 2H), 3.60-3.66 (m, 1H), 3.71-3.78 (m, 1H), 5.14 (s, 2H), 6.07 (d, 1H), 6.22 (dd, 1H), 6.58 (d, 1H), 7.38-7.55 (m, 6H), 7.98 (d, 1H). LRMS m/z (APCI) 411 [MH⁺]

### Example 10

### 4-[(4-Chlorobenzyl)oxy]-6′-[(3R)-3-hydroxypyrrolidin-1-yl]-2H-1,3′-bipyridin-2-one

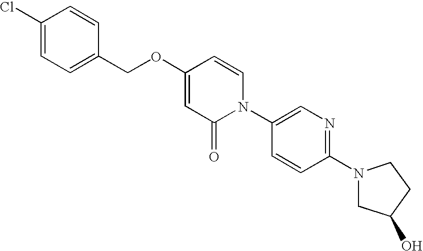

To a stirred solution of 4-hydroxy-6′-[(3R)-3-hydroxypyrrolidin-1-yl]-2H-1,3′-bipyridin-2-one from Preparation 4 (66 mg, 0.32 mmol) in acetonitrile (1 ml) was added 4-chlorobenzyl bromide (66 mg, 0.32 mmol) and cesium carbonate (105 mg, 0.32 mmol). The reaction mixture was heated at reflux for 6 hours. After cooling to room temperature, the reaction mixture was concentrated and the residue was partitioned between dichloromethane (5 ml) and water (5 ml). The organic component was separated using a phase separation cartridge and the resulting crude product mixture was purified by column chromatography (eluting with a gradient from EtOAc to 95:5:0.5 EtOAc:MeOH:NH₃) which gave the desired product as a white solid (51 mg, 44%). ¹H NMR (400 MHz, CD₃OD) δ ppm 2.01-2.09 (m, 1H), 2.12-2.21 (m, 1H), 3.41-3.48 (m, 1H), 3.58-3.63 (m, 3H), 4.50-4.52 (m, 1H), 5.12 (s, 2H), 6.05 (d, 1H), 6.22 (dd, 1H), 6.59 (d, 1H), 7.39-7.51 (m, 6H), 8.00 (d, 1H). LRMS m/z (APCI) 398 [MH⁺]

### Example 11

### 4-(Benzyloxy)-6′-[(3R)-3-hydroxypyrrolidin-1-yl]-2H-1,3′-bipyridin-2-one

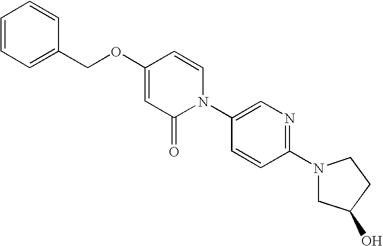

To a stirred solution of 4-(benzyloxy)-6′-fluoro-2H-1,3′-bipyridin-2-one from Preparation 2 (1.50 g, 5.06 mmol) in DMSO (5 ml) was added (R)-pyrrolidin-3-ol (1.32 g, 15.2 mmol; Aldrich). The reaction mixture was heated at 150° C. for 30 minutes. After cooling to room temperature the reaction mixture was diluted with water (20 ml) and a slight excess of saturated aqueous sodium bicarbonate solution was added. The solid was collected by filtration, washed with water (3×10 ml) and air-dried. The solid was heated at reflux in acetonitrile (40 ml), cooled to room temperature and the solid was collected by filtration. After washing with acetonitrile (10 ml) and air drying, the desired product was isolated as an off-white solid (1.66 g, 90%). ¹H NMR (400 MHz, CD₃OD) δ ppm 2.02-2.09 (m, 1H), 2.11-2.21 (m, 1H), 3.43-3.49 (m, 1H), 3.55-3.65 (m, 3H), 4.50-4.54 (m, 1H), 5.15 (s, 2H), 6.07 (d, 1H), 6.24 (dd, 1H), 6.58 (d, 1H), 7.31-7.53 (m, 7H), 7.98 (d, 1H). LRMS m/z (APCI) 364 [MH⁺].

### Example 12

### tert-Butyl {(3S)-1-[4-(benzyloxy)-2-oxo-2H-1,3′-bipyridin-6′-yl]pyrrolidin-3-yl}methylcarbamate

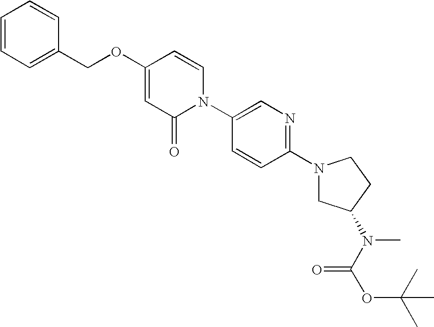

A mixture of 4-(benzyloxy)-6′-fluoro-2H-1,3′-bipyridin-2-one from Preparation 2 (500 mg, 1.69 mmol), (S)-methyl-pyrrolidinyl-3-yl-carbamic acid tert-butyl ester (372 mg, 1.86 mmol; ECA International) and potassium carbonate (700 mg, 5.06 mmol) in dimethylformamide (15 ml) was heated to 110° C. After 14 hours the reaction mixture was concentrated and the crude product mixture was purified by column chromatography eluting with ethyl acetate, followed by 9:1 ethyl acetate:methanol which gave the desired product as a white powder (693 mg, 86%). ¹H NMR (400 MHz, CD₃OD) δ ppm 1.45 (s, 9H), 2.21 (m, 2H), 2.81 (s, 3H), 3.43 (m, 2H), 3.84 (m, 2H), 4.82 (b, 1H), 5.15 (s, 2H), 6.08 (s, 1H), 6.25 (d, 1H), 6.05 (d, 1H), 7.31-7.45 (m, 7H), 8.02 (s, 1H). LRMS 477 [MH⁺].

### Example 13

### 4-(Benzyloxy)-6′-[(3S)-3-(methylamino)pyrrolidin-1-yl]-2H-1,3′-bipyridin-2-one dihydrochloride

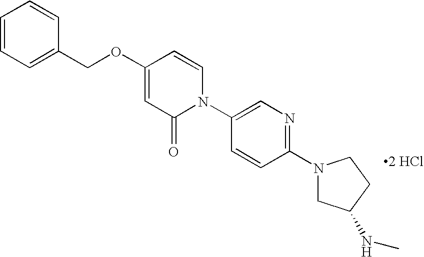

To a stirred solution of tert-butyl {(3S)-1-[4-(benzyloxy)-2-oxo-2H-1,3′-bipyridin-6′-yl]pyrrolidin-3-yl}methylcarbamate from example 12 (600 mg, 1.26 mmol) in dioxane (15 ml) was added 4M HCl in dioxane (20 ml, 80 mmol). After stirring for 15 hours at room temperature, the mixture was concentrated and air dried to give a light brown solid. The crude product was recrystallised from propan-2-ol/ethanol and dried to give the desired product as a light brown solid (350 mg, 50%). ¹H NMR (400 MHz, CD₃OD) δ ppm 2.45 (m, 1H), 2.65 (m, 1H), 2.85 (s, 3H), 3.85 (m, 1H), 3.94 (m, 2H), 4.14 (m, 2H), 5.18 (s, 2H), 6.08 (s, 1H), 6.31 (d, 1H), 7.21 (d, 1H), 7.31-7.45 (m, 5H), 7.58 (d, 1H), 8.08 (d, 1H), 8.19 (s, 1H). LRMS m/z (APCI) 377 [MH⁺]

### Example 14

### tert-Butyl {(3R)-1-[4-(benzyloxy)-2-oxo-2H-1,3′-bipyridin-6′-yl]pyrrolidin-3-yl}methylcarbamate

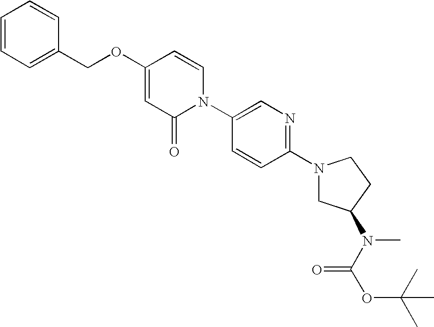

A mixture of 4-(benzyloxy)-6′-fluoro-2H-1,3′-bipyridin-2-one from Preparation 2 (500 mg, 1.69 mmol), (R)-methyl-pyrrolidinyl-3-yl-carbamic acid tert-butyl ester (372 mg, 1.86 mmol; see Example 34b in WO2003/106462) and potassium carbonate (700 mg, 5.06 mmol) in N,N-dimethylformamide (15 ml) was heated to 110° C. After 14 hours the reaction mixture was concentrated and the crude product mixture was purified by column chromatography eluting with ethyl acetate, followed by 9:1 ethyl acetate:methanol which gave the desired product as a white powder (693 mg, 86%). ¹H NMR (400 MHz, CD₃OD) δ ppm 1.45 (s, 9H), 2.21 (m, 2H), 2.81 (s, 3H), 3.43 (m, 2H), 3.84 (m, 2H), 4.82 (b, 1H), 5.15 (s, 2H), 6.08 ((s, 1H), 6.25 (d, 1H), 6.05 (d, 1H), 7.31-7.45 (m, 7H), 8.02 (s, 1H). LRMS m/z 477 [MH⁺]

### Example 15

### 4-(benzyloxy)-6′-[(3R)-3-(methylamino)pyrrolidin-1-yl]-2H-1,3′-bipyridin-2-one dihydrochloride

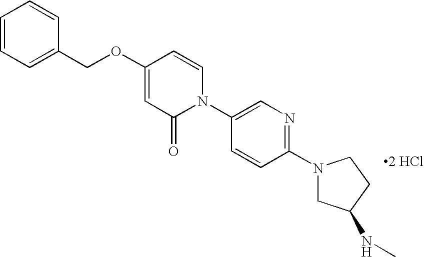

To a stirred solution of tert-butyl {(3R)-1-[4-(benzyloxy)-2-oxo-2H-1,3′-bipyridin-6′-yl]pyrrolidin-3-yl}methylcarbamate from Example 14 (650 mg, 1.36 mmol) in dioxane (15 ml) was added 4M HCl in dioxane (27 ml, 109 mmol). After stirring for 15 hours at room temperature, the mixture was concentrated and air dried to give a light brown solid. The crude product was recrystallised from propan-2-ol/ethanol and dried to give the desired product as a light brown solid (430 mg, 70%). ¹H NMR (400 MHz, CD₃OD) δ ppm 2.45 (m, 1H), 2.65 (m, 1H), 2.85 (s, 3H), 3.85 (m, 1H), 3.94 (m, 2H), 4.14 (m, 2H), 5.18 (s, 2H), 6.08 (s, 1H), 6.31 (d, 1H), 7.21 (d, 1H), 7.31-7.45 (m, 5H), 7.58 (d, 1H), 8.08 (d, 1H), 8.19 (s, 1H). LRMS m/z (APCI) 377 [MH⁺].

### Example 16

### 4-(benzyloxy)-6′-[(3S)-3-hydroxypyrrolidin-1-yl]-2H-1,3′-bipyridin-2-one

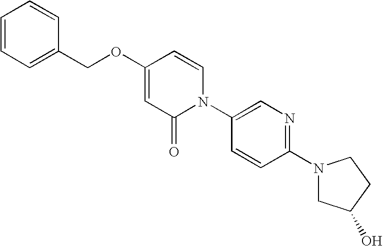

A stirred solution of 4-benzyloxy-1H-pyrid-2-one (25 mg, 0.13 mmol), (3S)-1-(5-Bromopyridin-2-yl)pyrrolidin-3-ol from Preparation 6 (30 mg, 0.13 mmol), trans-1,2-diaminocyclohexane (2 μl, 0.02 mmol), potassium carbonate (17 mg, 0.13 mmol) and copper(I) iodide (28 mg, 0.13 mmol) in N-methylpyrrolidinone (1 ml) was heated at 150° C. for 15 hours in a reacti-vial. After cooling to room temperature, the mixture was diluted with DCM (10 ml), filtered through celite and the filtrate was washed with water (5 ml). The aqueous phase was extracted with DCM (2×5 ml) and the combined organic layers were passed through a phase separation cartridge. The crude product mixture was purified by preparative HPLC (C₁₈ Phenomenex® luna 10μ C18(2) 100 Å; mobile phase, gradient using H₂O with 0.1% formic acid and MeCN with 0.1% formic acid) to give the desired product as a brown solid (5 mg, 17%). ¹H NMR (400 MHz, CD₃OD) δ ppm 2.01-2.09 (m, 1H), 2.12-2.21 (m, 2H), 3.41-3.48 (m, 1H), 3.58-3.63 (m, 3H), 4.50-4.52 (m, 1H), 6.05 (d, 1H), 6.15 (dd, 1H), 6.62 (d, 1H), 7.32-7.51 (m, 6H), 7.53 (dd, 1H), 8.00 (d, 1H). LRMS m/z (APCI) 364 [MH⁺]

### Example 17a

### 6′-(3-Amino-3-methyl pyrrolidin-1-yl)-4-(benzyloxy)-2H-1,3′-bipyridin-2-one (enantiomer 1)

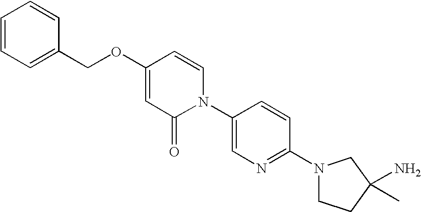

A solution of protected amine of Preparation 8a (29 mg, 0.06 mmol) in dichloromethane (5 ml) was cooled with an ice bath and treated with trifluoroacetic acid (2 ml). The cold bath was removed and the solution was stirred at room temperature for 4 hours. The solution was concentrated in vacuo, the residue was dissolved in methanol and loaded onto a SCX-2 cartridge. Non basic products were eluted with methanol and the desired product was eluted with 2N NH3/MeOH and concentrated in vacuo to give the title compound as a colourless solid 21 mg (91%). ¹H NMR (400 MHz, CD₃OD) δ ppm 1.35 (s, 3H) 1.99 (t, 2H) 3.40 (s, 2H) 3.53-3.59 (m, 1H) 3.63-3.69 (m, 1H) 5.14 (s, 2H) 6.07 (d, 1H) 6.24 (dd, 1H) 6.55 (d, 1H) 7.34-7.50 (m, 7H) 7.97 (d, 1H). LRMS m/z (APCI) 377 [MH⁺]

### Example 17b

### 6′-(3-Amino-3-methyl pyrrolidin-1-yl)-4-(benzyloxy)-2H-1,3′-bipyridin-2-one (enantiomer 2)

Prepared using the same method as 17a from Preparation 8b to give the title compound as a colourless solid. ¹H NMR (400 MHz, CD₃OD) δ ppm 1.35 (s, 3H) 1.99 (t, 2H) 3.40 (s, 2H) 3.53-3.59 (m, 1H) 3.63-3.69 (m, 1H) 5.14 (s, 2H) 6.07 (d, 1H) 6.24 (dd, 1H) 6.55 (d, 1H) 7.34-7.50 (m, 7H) 7.97 (d, 1H). LRMS m/z (APCI) 377 [MH⁺].

### Example 18a

### 4-(Benzyloxy)-6′-[3-methyl-3-(methylamino)pyrrolidin-1-yl]-2H-1,3′-bipyridin-2-one (enantiomer 1)

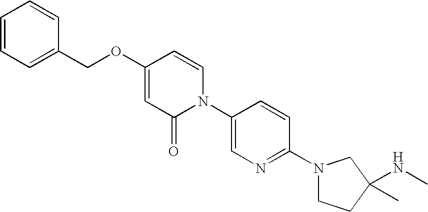

A solution of protected amine of Preparation 9a (50 mg, 0.1 mmol) in dichloromethane (3 ml) was cooled with an ice bath and treated with trifluoroacetic acid (2 ml). The cold bath was removed and the solution was stirred at room temperature for 4 hours. The solution was concentrated in vacuo and the residue was dissolved in methanol and loaded onto a SCX-2 cartridge. Non basic products were eluted with methanol and the desired product was eluted with 2N NH3/MeOH and concentrated in vacuo to give the title compound as a colourless solid 29 mg (72%). ¹H NMR (400 MHz, CD₃OD) δ ppm 1.30 (s, 3H) 1.95-2.00 (m, 1H) 2.06-2.13 (m, 1H) 2.37 (s, 3H) 3.36-3.55 (m, 3H) 3.60-3.66 (m, 1H) 5.14 (s, 2H) 6.07 (d, 1H) 6.24 (dd, 1H) 6.56 (d, 1H) 7.34-7.51 (m, 7H) 7.98 (d, 1H). LRMS m/z (APCI) 391 [MH⁺]

### Example 18b

### 4-(Benzyloxy)-6′-[3-methyl-3-(methylamino)pyrrolidin-1-yl]-2H-1,3′-bipyridin-2-one (enantiomer 2)

Prepared using the same method as 18a from preparation 9b to give the title compound as a colourless solid. ¹H NMR (400 MHz, CD₃OD) δ ppm 1.30 (s, 3H) 1.95-2.00 (m, 1H) 2.06-2.13 (m, 1H) 2.37 (s, 3H) 3.36-3.55 (m, 3H) 3.60-3.66 (m, 1H) 5.14 (s, 2H) 6.07 (d, 1H) 6.24 (dd, 1H) 6.56 (d, 1H) 7.34-7.51 (m, 7H) 7.98 (d, 1H). LRMS m/z (APCI) 391 [MH⁺]

### Example 19

### 4-(Benzyloxy)-6′-[(3R)-3-(ethylamino)pyrrolidin-1-yl]-2H-1,3′-bipyridin-2-one

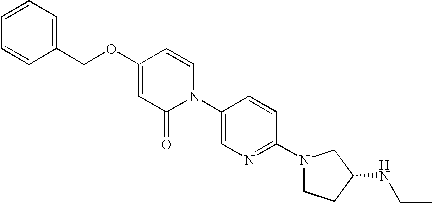

Hydrogen chloride (4M in Dioxane) (6.52 mL, 26.1 mmol) was added to a solution of the pyrrolidine of Preparation 11a (640 mg, 1.30 mmol) in dichloromethane (10 ml) at room temperature under N₂(g). The reaction mixture was stirred for 4 hours. Solvents were removed in vacuo to give a dark brown solid (760 mg). The residue was dissolved in methanol and loaded onto a SCX-2 cartridge. Non basic products were eluted with methanol and the desired product was eluted with 2N NH3/MeOH and concentrated in vacuo to give the title compound as a brown solid. Further purification by column chromatography (eluting with DCM:MeOH:NH3 99:1:0.1→95:5:0.5) gave the title compound as a pale brown solid 500 mg (98%). ¹H NMR (400 MHz, CDCl₃) δ ppm 1.17 (t, 3H), 1.86-1.94 (m, 1H), 2.20-2.29 (m, 1H), 2.78 (q, 2H), 3.31 (dd, 1H), 3.43-3.57 (m, 2H), 3.60-3.67 (m 1H), 3.75 (dd, 1H), 5.02 (s, 2H), 6.01-6.02 (m, 2H), 6.41 (d, 1H), 7.19 (d, 1H), 7.32-7.45 (m, 5H), 7.45 (dd, 1H), 8.02 (s, 1H). LRMS m/z (ESI)=391 [MH+]

### Example 20

### N-{(3S)-1-[4-(Benzyloxy)-2-oxo-2H-1,3′-bipyridin-6′-yl]pyrrolidin-3-yl}-N-methylacetamide

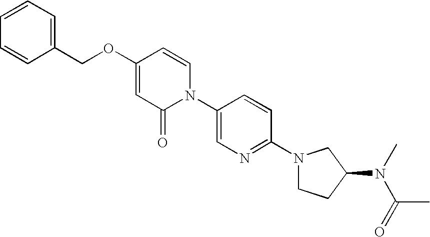

A solution of amine of Example 13 (7.84 g, 20.8 mmol) in dichloromethane (70 ml) was treated with triethylamine (8.7 ml, 62.5 mmol) and cooled with an ice bath. Acetic anhydride (2.95 ml, 31.2 mmol) was added and the resulting solution was stirred at room temperature overnight under nitrogen. The solution was diluted with saturated aqueous potassium carbonate (50 ml) and stirred at room temperature for 1 hour. The layers were separated, the organics were washed with brine (5 ml), dried (Na₂SO₄), filtered and concentrated in vacuo to a colourless oil that solidified to a white solid on standing. Trituration with ether gave the title compound as a colourless solid. ¹H NMR (400 MHz, CDCl₃) δ ppm ¹H-NMR (Variable temperature, 90° C., DMSO-d₆): 2.07 (s, 3H) 2.08-2.24 (m, 2H) 2.86 (br. s., 3H) 3.36-3.46 (m, 2H) 3.62-3.70 (m, 2H) 4.82-5.10 (br. m, weak, 1H), 5.15 (s, 2H), 5.94 (d, 1H), 6.07 (dd, 1H), 6.54 (d, 1H), 7.34-7.51 (m, 7H), 8.03 (d, 1H) LRMS m/z (ESI)=419 [MH+]

### Example 21, 21a, 21b

### =Preparation 8, 8a, 8b

### tert-Butyl {1-[4-(benzyloxy)-2-oxo-2H-1,3′-bipyridin-6′-yl]-3-methyl pyrrolidin-3-yl}carbamate

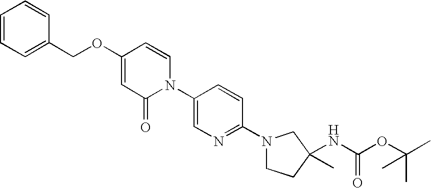

A solution of 4-benzyloxy-2(1H)-pyridone (186 mg, 0.92 mmol), the iodide of preparation 7 (373 mg, 0.92 mmol), copper iodide (176 mg, 0.92 mmol), potassium carbonate (256 mg, 1.85 mmol) and rac-trans-N,N′-dimethyl cyclohexane-1,2-diamine (146 ml, 0.92 mmol) in 1,4-dioxan (5 ml) was degassed via a nitrogen/vacuum cycle. The resulting mixture was the heated to 110° C. for 16 hours. The reaction was cooled to room temperature and concentrated in vacuo. The residue was treated with 0.880NH₃/H₂o (10 ml, 1:1 ratio) and stirred for 15 min. The resulting solid was extracted with ethyl acetate (2×20 ml). The combined organics were washed with brine (20 ml), dried (Na₂SO₄), filtered and concentrated in vacuo to give a brown oil. Purification by column chromatography (eluting with 100% DCM→95:5:0.5) gave the title compound (Prep8/Ex. 21 as racemate) as a pale yellow solid, 307 mg (69%). ¹H NMR (400 MHz, CD₃OD) δ ppm 1.43 (s, 9H), 1.48 (s, 3H), 1.96-2.04 (m, 1H), 2.33-2.41 (m, 1H), 3.42-3.58 (bm, 3H), 3.81-3.84 (m, 1H), 5.15 (s, 2H), 6.08 (d, 1H), 6.25 (dd, 1H), 6.55 (d, 1H), 7.32-7.51 (bm, 7H), 7.97 (d, 1H). LRMS m/z (APCI) 477 [MH⁺].

Chiral prep HPLC (Chiralcel OJ-H, 250×21.2 mm id, 100% MeOH, 20 ml/min rt) enabled separation of the individual enantiomers

Peak 1: Retention time 9.72 minutes Prep. 8a/Ex. 21a

Peak 2: Retention time 15.14 minutes Prep. 8b/Ex. 21b

### Example 22a; 22b

### =Preparation 9a; 9b

### tert-Butyl {1-[4-(benzyloxy)-2-oxo-2H-1,3′-bipyridin-6′-yl]-3-methylpyrrolidin-3-yl}methyl carbamate—single enantiomer

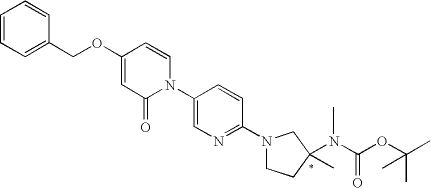

A solution of the pyrrolidinone of preparation 8a (57 mg, 0.12 mmol) in DMF (2 ml) was cooled in an ice bath and treated with sodium hydride (7.2 mg of a 60% dispersion in mineral oil, 0.12 mmol) under a nitrogen atmosphere. The mixture was allowed to warm gradually to room temperature over 2 hours before iodomethane (9 [symbol] l, 0.14 mmol) was added. Once added, the solution was stirred at room temperature overnight. The reaction mixture was quenched with a few drops of water, diluted with methanol and loaded onto a SCX-2 cartridge, rinsing with methanol. The desired product was eluted with 2N NH3/MeOH and concentrated in vacuo to give the title compound (Prep. 9a/Ex. 22a) as a colourless oil, 50 mg (85%). ¹H NMR (400 MHz, CD₃OD) δ ppm 1.34 (s, 3H) 1.50 (s, 9H) 2.23-2.37 (m, 2H) 2.93 (s, 3H) 3.39-3.45 (m, 1H) 3.56 (d, 1H) 3.62 (t, 1H) 4.00 (d, 1H) 5.14 (s, 2H) 6.07 (d, 1H) 6.24 (dd, 1H) 6.54 (d, 1H) 7.32-7.52 (m, 7H) 8.00 (d, 1H). LRMS m/z (APCI) 491 [MH⁺].

The opposite enantiomer Prep. 9b (Example 22b) was prepared via the same method with preparation 8b as starting material to give the title compound as a colourless oil. ¹H NMR (400 MHz, CD₃OD) δ ppm 1.34 (s, 3H) 1.50 (s, 9H) 2.23-2.37 (m, 2H) 2.93 (s, 3H) 3.39-3.45 (m, 1H) 3.56 (d, 1H) 3.62 (t, 1H) 4.00 (d, 1H) 5.14 (s, 2H) 6.07 (d, 1H) 6.24 (dd, 1H) 6.54 (d, 1H) 7.32-7.52 (m, 7H) 8.00 (d, 1H). LRMS m/z (APCI) 491 [MH⁺].

### Example 23a; 23b

### =Preparation 11a; 11b

### tert-Butyl {(3R)-1-[4-(benzyloxy)-2-oxo-2H-1,3′-bipyridin-6′-yl]pyrrolidin-3-yl}ethylcarbamate

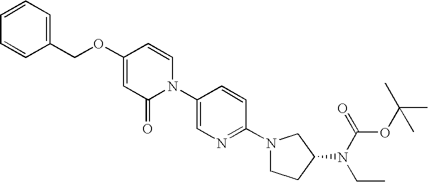

A solution of 4-benzyloxy-2(1H)-pyridone (274 mg, 1.36 mmol), the iodide of preparation 10 (567 mg, 1.36 mmol), copper iodide (53 mg, 0.27 mmol), potassium carbonate (376 mg, 2.72 mmol) and rac-trans-N,N′-dimethyl cyclohexane-1,2-diamine (86 ml, 0.54 mmol) in 1,4-dioxan (10 ml) was degassed via a nitrogen/vacuum cycle. The resulting mixture was then heated to 100° C. under a nitrogen atmosphere for 16 hours. The reaction was cooled to room temperature and concentrated in vacuo. The residue was treated with 0.880NH₃/H₂O (10 ml, 1:1 v/v ratio) and stirred for 15 min. The resulting solid was filtered off and dried in vacuo giving the title compound (Prep. 11a/Ex. 23a) as a brown solid 550 mg (82%). ¹H NMR (400 MHz, CDCl₃) δ ppm 1.13 (t, 3H), 1.43 (s, 9H). 2.09-2.25 (m, 2H), 3.12-3.30 (m, 2H), 3.31 (t, 1H), 3.40 (q, 1H), 3.65 (q, 2H), 4.60-4.77 (m, 1H), 5.01 (s, 2H), 6.00-6.01 (m, 2H), 6.40 (d, 1H), 7.18 (d, 1H), 7.30-7.41 (m, 5H), 7.48 (dd, 1H), 8.03 (d, 1H). LRMS m/z (ESI) 491 [MH+].

The opposite enantiomer Prep. 11b (Example 23b) was prepared via the same method with preparation 10b as starting material to give the title compound as a brown solid. 1H NMR (400 MHz, CDCl₃) δ ppm 1.13 (t, 3H), 1.43 (s, 9H). 2.09-2.25 (m, 2H), 3.12-3.30 (m, 2H), 3.31 (t, 1H), 3.40 (q, 1H), 3.65 (q, 2H), 4.60-4.77 (m, 1H), 5.01 (s, 2H), 6.00-6.01 (m, 2H), 6.40 (d, 1H), 7.18 (d, 1H), 7.30-7.41 (m, 5H), 7.48 (dd, 1H), 8.03 (d, 1H). LRMS m/z (ESI) 491 [MH+]

### Example 25, 25a, 25b

### 1-[4-(Benzyloxy)-2-oxo-2H-1,3′-bipyridin-6′-yl]-N,N-dimethylpyrrolidine-3-carboxamide

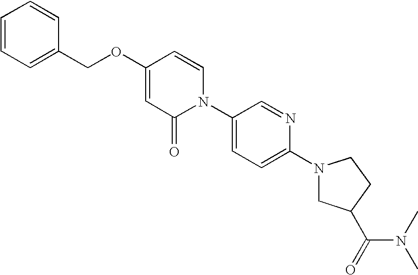

Prepared according to Preparation 28 from 1-[4-(Benzyloxy)-2-oxo-2H-1,3′-bipyridin-6′-yl]pyrrolidine-3-carboxylic acid from Preparation 31 and dimethylamine-hydrochloride to to give the title compound (Ex. 25 as a racemate) as a colourless solid. ¹H NMR (400 MHz, CDCl₃) δ ppm 2.19-2.26 (m, 1H), 2.33-2.44 (m, 1H), 2.98 (s, 3H), 3.10 (s, 3H), 3.36-3.43 (m, 1H), 3.46 (q, 1H), 3.65-3.73 (m, 2H), 3.81 (t, 1H), 5.01 (s, 2H), 6.01-6.02 (m, 2H), 6.42 (d, 1H), 7.19 (d, 1H), 7.33-7.43 (m, 5H), 7.48 (dd, 1H), 8.03 (d, 1H). LRMS m/z (ESI) 419 [MH⁺]

Chiral prep HPLC (Chiralpak OJ-H, 250×21.2 mm id, 100% MeOH, 18 ml/min r.t.) enabled separation of the individual enantiomers.

Peak 1: Retention time 17.41 minutes, Ex. 25a Rotation: [α]D25=−15.16o (3.10 mg/mL MeOH)

Peak 2: Retention time 28.94 minutes, Ex. 25b Rotation: [α]_{D}²⁵=19.62° (3.15 mg/mL MeOH)

### Example 26

### 4-(Benzyloxy)-6′-[(3aR,6aS)-5-ethyl hexahydropyrrolo[3,4-c]pyrrol-2(1H)-yl]-2H-1,3′-bipyridin-2-one

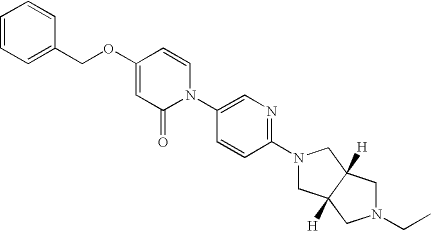

4-(Benzyloxy)-6′-[(3aR,6aS)-hexahydropyrrolo[3,4-c]pyrrol-2(1H)-yl]-2H-1,3′-bipyridin-2-one dihydrochloride from Example 8 (95 mg, 0.206 mmol) was suspended in dichloromethane (2.00 ml) and triethylamine (0.057 ml, 0.41 mmol) was added. The solution was stirred at r.t. under N₂ for a few minutes until dissolution was effective. Acetaldehyde (0.115 mL, 2.06 mmol) was then added and the reaction mixture stirred for 10 minutes before adding sodium triacetoxyborohydride (66 mg, 0.31 mmol) in one portion. The opaque yellow solution was then stirred overnight. MS suggested product formation. K₂CO₃ (aq) (20 ml) was added and the reaction mixture was stirred for 30 minutes. The resultant layers were then separated and the organic layer was dried (MgSO₄), filtered and evaporated to give crude product as an orange residue. The residue was purified by ISCO chromatography (4 g cartridge) eluting with DCM to DCM:MeOH:NH₃ 95:5:0.5 over 15 minutes to give the title compound as an orange. (16 mg, 19%) ¹H NMR (400 MHz, CDCl₃) δ ppm 0.09 (t, 3H), 2.40 (dd, 2H), 2.49 (q, 2H), 2.87-3.03 (m, 4H), 3.43 (dd, 2H), 3.61 (dd, 2H), 5.01 (s, 2H), 6.01-6.02 (m, 2H), 6.43 (d, 1H), 7.19 (d, 1H), 7.31-7.42 (m, 5H), 7.49 (dd, 1H), 8.05 (s, 1H). LRMS m/z (APCI & ESI) 417 [MH⁺]

### Example 27

### 4-(Benzyloxy)-6′-[(3′S)-(2-oxo-1,3′-bipyrrolidin-1′-yl]-2H-1,3′bipyridin-2-one

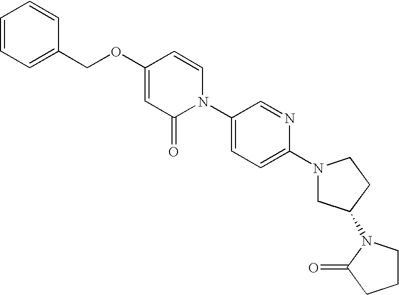

N-{(3S)-1-[4-(Benzyloxy)-2-oxo-2H-1,3′-bipyridin-6′-yl]pyrrolidin-3-yl}-4-chlorobutanamide from Preparation 54 (120 mg, 0.26 mmol) was added to a solution of sodium hydride (60% dispersion in mineral oil, 25 mg, 0.65 mmol) in NMP (5 ml) and the RM was stirred at r.t. for 16 hours. MS showed evidence for product and no SM. TLC (EtOAc:MeOH:NH₃ 90:10:1) showed both product and SM remaining. A further 0.5 eq of NaH (60% disp in oil, 10 mg) was added and the solution was warmed to 40° C. for 4 hours then cooled to r.t. overnight. The reaction mixture was quenched with sat NH₄Cl (aq) (20 ml) then diluted with EtOAc (30 ml). Layers were separated and the organic layer was washed with brine (30 ml) before drying (MgSO₄), filtered and evaporated to give a yellow oil. The oil was purified by cartridge chromatography (20 g cartridge) eluting with EtOAc:MeOH:NH₃ 98:2:0.2 (100 mL) to 96:4:0.4 (100 mL) to 96:5:0.5 (200 mL) to 93:7:0.7 (300 mL) which gave the title compound as a colorless oil which crystallised on standing (44.0 mg, 39%) ¹H NMR (400 MHz, CDCl₃) δ ppm 2.00-2.07 (m, 2H), 2.09-2.18 (m, 1H), 2.25-2.35 (m, 1H), 2.42 (t, 2H), 3.40 (t, 2H), 3.45-3.56 (m, 2H), 3.63-3.73 (m, 2H), 4.89-4.95 (m, 1H), 5.03 (s, 2H), 6.02-6.03 (m, 2H), 6.42 (d, 1H), 7.19-7.21 (d, 1H), 7.32-7.42 (m, 5H), 7.52 (dd, 1H), 8.03 (d, 1H). LRMS m/z (ESI) 431 [MH+]

### Example 28

### (3S)-1-{4-[(4-chlorobenzyl)oxy]-2-oxo-2H-1,3′-bipyridin-6′-yl}pyrrolidin-3-yl acetate

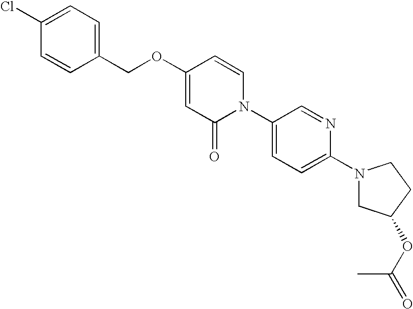

(3R)-1-{4-[(4-chlorobenzyl)oxy]-2-oxo-2H-1,3′-bipyridin-6′-yl}pyrrolidin-3-yl methanesulfonate from Preparation 5 (20 mg, 0.042 mmol) and potassium acetate (4.12 mg, 0.042 mmol) in THF (3 ul, 0.042 mmol) was stirred in a reactive vial at 100° C. for 64 hours. MS indicated still some starting material remaining. The mixture was stirred at r.t. over a week. The mixture was evaporated. LC/MS and MS indicated completion of the reaction. The residue was partitioned between DCM (5 mL) and 10% K₂CO₃(aq) (5 mL). The organics were dried over a separating phase cartridge and columned using ISCO (4 g column) to give the title compound (7 mg, 38%) assume to be this stereochemistry (via SN2) ¹H NMR (400 MHz, CD₃OD) δ ppm 2.04 (s, 3H), 2.16-2.40 (m, 2H), 3.52-3.70 (m, 3H), 3.75 (dd, 1H), 5.15 (s, 2H), 5.39-5.47 (m, 1H), 6.08 (d, 1H), 6.25 (d, 1H), 6.63 (d, 1H), 7.22-7.64 (m, 6H), 8.01 (d, 1H). LRMS m/z (APCI & ESI) 440 [MH⁺]

### Example 29

### 4-[(4-Chlorobenzyl)oxy]-6′-[(3S)-3-hydroxypyrrolidin-1-yl]-2H-1,3′-bipyridin-2-one

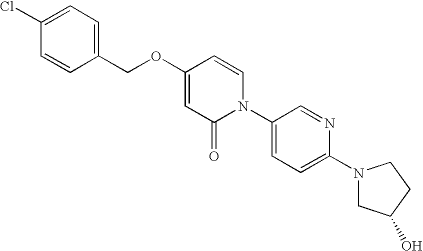

(3S)-1-{4-[(4-chlorobenzyl)oxy]-2-oxo-2H-1,3′-bipyridin-6′-yl}pyrrolidin-3-yl acetate from Example 28 (7 mg, 0.0159 mmol) and K₂CO₃ (11 mg, 0.796 mmol) were stirred in methanol at r.t. overnight. TLC, MS and LCMS indicated a bit of starting material not consumed. 5 Eq of K₂CO₃ were added and the mixture was stirred at r.t. for 2 h. TLC and MS indicated completion of the reaction. The mixture was diluted with DCM (2 ml). The organics were washed with water (1 ml) and dried over a separating phase cartridge to give the title compound as a white solid (5 mg, 79%). ¹H NMR (400 MHz, CD₃OD) δ ppm 2.06 (m, 1H), 2.14 (m, 1H), 3.46 (d, 1H), 3.53-3.64 (m, 3H), 4.53 (m, 1H), 5.15 (s, 2H), 6.07 (d, 1H), 6.23 (dd, 1H), 6.58 (d, 1H), 7.31-7.55 (m, 7H), 7.98 (d, 1H). LRMS m/z (APCI & ESI) 398 [MH⁺]

### Example 30

### 4-(Benzyloxy)-6′-(3-oxopyrrolidin-1-yl)-2H-1,3′-bipyridin-2-one

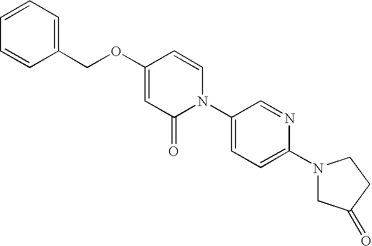

To a stirred solution of oxalyl chloride (622 ul, 7.40 mmol) in dichloromethane (anhydrous, 25 ml) at −78° C. was added anhydrous dimethyl sulphoxide (788 ul, 11.1 mmol). After 20 min a solution of 4-(Benzyloxy)-6′-[(3R)-3-hydroxypyrrolidin-1-yl]-2H-1,3′-bipyridin-2-one from Example 11 (1.92 g, 5.28 mmol) in anhydrous dichloromethane (25 ml) was added (the alcohol was added as a fine suspension after sonication). After 45 min triethylamine (2.95 ml, 21.1 mmol)) was added and the reaction was allowed to warm up to room temperature. The reaction was quenched with water and extracted with dichloromethane (2×50 ml). The combined organics were washed with sat. sodium bicarbonate solution (50 ml), brine (2×50 ml), dried (Na₂SO₄) and concentrated in vacuo. The residue was purified by column chromatography (Redisep 40 g) eluting with DCM/MeOH/NH₃ (100/0/0->97/3/0.3) to give the title compound as a white solid (800 mg, 42%) ¹H NMR (400 MHz, d₆-DMSO) δ ppm 2.67-2.74 (m, 2H), 3.76-3.86 (m, 4H), 5.12 (s, 2H), 5.94 (m, 1H), 6.04-6.10 (m, 1H), 6.62-6.68 (m, 1H), 7.31-7.68 (m, 7H), 8.07 (m, 1H). LC-MS RT=2.58 min m/z (APCI & ESI) 362 [MH⁺] (6 min acidic run)

### Example 32

### =Preparation 53

### tert-Butyl methyl{(3S)-1-[2-oxo-4-(2-phenylethyl)-2H-1,3′-bipyridin-6′-yl]pyrrolidin-3-yl}-carbamate

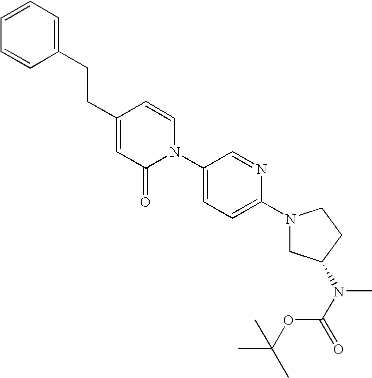

To a stirred solution of 6′-{(3S)-3-[(tert-Butoxycarbonyl)(methyl)amino]pyrrolidin-1-yl}-2-oxo-2H-1,3′-bipyridinyl-4-yl trifluoromethanesulfonate from Preparation 52 (50 mg, 0.096 mmol) in THF (784 uL) was added NMP (139 uL), phenethylzinc bromide (289 uL, 0.145 mmol) and bis(tri-t-butylphosphine)palladium(0) (1.01 mg, 0.002 mmol) and the reaction mixture was stirred at 50° C. overnight. The reaction mixture was cooled to r.t., diluted with EtOAc (5 mL), washed with water (3×5 ml). The organics were dried (Na₂SO₄) and evaporated in vacuo. The residue was purified by column chromatography using ISCO (4 g column) and eluant EtOAc/MeOH/NH₃ from EtOAc only to 95/5/0.5 to give the title compound as a colourless solid (12 mg, 27%). ¹H-NMR (400 MHz, CD₃OD) δ ppm 1.42 (s, 9H), 2.17-2.21 (m, 2H), 2.81-2.86 (m, 5H), 2.90-2.95 (m, 2H), 3.39-3.42 (m, 2H), 3.62-3.69 (m, 2H), 4.78-4.82 (m, 1H), 6.37-6.39 (m, 2H), 6.59 (d, 1H), 7.11-7.26 (m, 5H), 7.42 (d, 1H), 7.51 (dd, 1H), 7.98 (d, 1H). LRMS m/z (APCI & ESI) 475 [MH⁺]

### Example 33

### =Preparation 56

### 4-(Benzyloxy)-5′-[(3R)-3-(methylamino)pyrrolidin-1-yl]-2H-1,2′-bipyridin-2-one

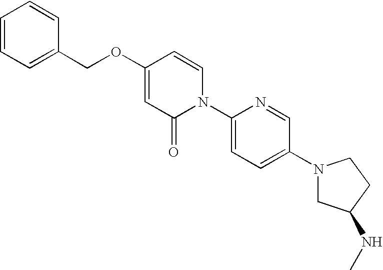

Prepared according to Example 17a from tert-Butyl {(3R)-1-[4-(benzyloxy)-2-oxo-2H-1,2′-bipyridin-5′-yl]pyrrolidin-3-yl}methylcarbamate from Preparation 55 to give the title compound as a brownish solid. ¹H NMR (400 MHz, d₆-DMSO) δ ppm 1.90-1.95 (m, 1H), 2.16-2.21 (m, 1H), 2.41 (s, 3H), 3.18-3.52 (m, 5H), 5.13 (s, 2H), 5.94 (d, 1H), 6.10 (dd, 1H), 7.04 (dd, 1H), 7.36-7.46 (m, 6H), 7.68 (d, 1H), 7.80 (d, 1H). LRMS m/z (FIA) 377 [MH⁺]

Examples 34-124 were prepared according to the methods described above for examples 1, 3 and 11, or routine variation thereof, starting from the appropriate 2-fluoropyridine¹ and the appropriate amine²
1. 2-Fluoropyridines are described for example in preparations 2, 13 and 15.

2. The required amines are either commercially available, described in the literature or in preparations 17, 18, 20 and 21, or represent a routine modification thereof. “De-Bocylation” is generally carried out where necessary according to the procedure of Example 13.

| | |
|---|---|
| | I |
| 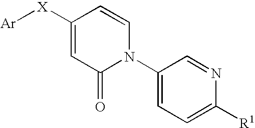 | |

| Example | Ar—X | R¹ | Analytical Data | Preparation |
|---|---|---|---|---|
| 34 | 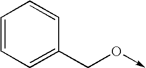 | 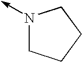 | LC-MS RT = 2.17 min m/z (ESI) 348 [MH⁺](6 min run) | As Example 1 in n-butanol at 80° C. |
| 35 | 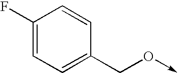 | 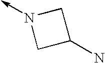 | LC-MS RT = 2.57 min m/z (APCI & ESI) 367 [MH⁺](6 min basic run) | As Example 1 but with Et₃N, NMP followed by Example 13 |
| 36 | 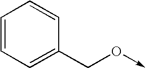 | 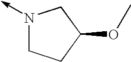 | LC-MS RT = 3.04 min m/z (APCI) 378 [MH⁺](6 min run) | As Example 3 |
| 37 | 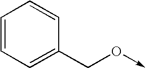 | 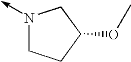 | ¹H NMR (400 MHz, CDCl₃) δ ppm 2.01-2.18 (m, 2 H), 3.32 (s, 3 H), 3.46-3.60 (m, 4 H), 4.01-4.04 (m, 1 H), 4.99 (s, 2 H), 6.00- 6.02 (m, 2 H), 6.39 (d, 1 H), 7.17 (dd, 1 H), 7.23- 7.40 (m, 5 H), 7.53 (dd, 1 H), 8.01 (s, 1 H). LRMS m/z (APCI) 378 [MH⁺] | As Example 3 |
| 38a Enantiomer a | 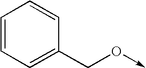 | 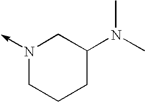 | ¹H NMR (400 MHz, CDCl₃) δ ppm 1.43-1.61 (m, 2 H), 1.80-1.85 (m, 1 H), 2.01-2.07 (m, 1 H), 2.49-2.53 (m, 1 H) 2.40 (s, 6 H), 2.63 (t, 2 H), 4.21 (d, 1 H), 4.43 (d, 1 H), 5.02 (s, 2 H), 6.02 (d, s, 2 H), 6.69 (d, 1 H), 7.18 (dd, 1 H), 7.35-7.42 (m, | |
| # 5 H), 7.50 (dd, 1 H), 8.03 (s, 1 H). LRMS m/z (APCI) 405 [MH⁺], [α]_{D}²⁵ +28.00, 2.00 mg/ml MeOH) | As Example 3 | | | |
| 38b Enantiomer b | 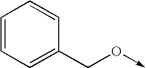 | 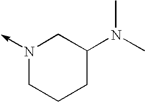 | ¹H NMR (400 MHz, CDCl₃) δ ppm 1.43-1.61 (m, 2 H), 1.80-1.85 (m, 1 H), 2.01-2.07 (m, 1 H), 2.49-2.53 (m, 1 H) 2.40 (s, 6 H), 2.63 (t, 2 H), 4.21 (d, 1 H), 4.43 (d, 1 H), 5.02 (s, 2 H), 6.02 (d, s, 2 H), 6.69 (d, 1 H), 7.18 (dd, 1 H), 7.35-7.42 (m, | |
| # 5 H), 7.50 (dd, 1 H), 8.03 (s, 1 H). LRMS m/z (APCI) 405 [MH⁺], [α]_{D}²⁵ −26.41, (1.95 mg/ml MeOH) | As Example 3 | | | |
| 39 | 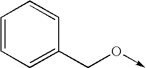 | 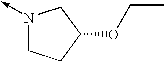 | ¹H NMR (400 MHz, CDCl₃) δ ppm 1.20 (t, 3 H), 2.05- 2.18 (m, 2 H), 3.42-4.61 (m, 6 H), 4.17-4.21 (m, 1 H), 5.01 (s, 2 H), 6.01- 6.03 (m, 2 H), 6.40 (d, 1 H), 7.19 (dd, 1 H), 7.33- 7.42 (m, 5 H), 7.49 (dd, 1 H), 8.03 (s, 1 H). LRMS m/z (APCI) 392 [MH⁺], | |
| # [α]_{D}²⁵ −24.00, (2.5 mg/mL MeOH) | As Example 3 | | | |
| 40 | 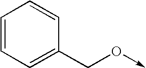 | 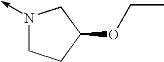 | ¹H NMR (400 MHz, CDCl₃) δ ppm 1.20 (t, 3 H), 2.05-2.18 (m, 2 H), 3.42-4.61 (m, 6 H), 4.17- 4.21 (m, 1 H), 5.01 (s, 2 H), 6.01-6.03 (m, 2 H), 6.40 (d, 1 H), 7.19 (dd, 1 H), 7.33-7.42 (m, 5 H), 7.49 (dd, 1 H), 8.03 (s, 1 H). LRMS m/z (APCI) 392 [MH⁺] | As Example 3 |
| 41a Enantiomer a | 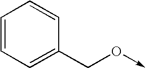 | 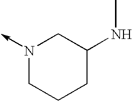 | LC-MS RT = 2.05 min m/z (APCI) 391 [MH⁺](6 min run) | As Example 3 |
| 41b Enantiomer b | 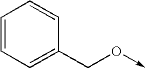 | 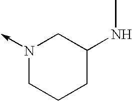 | LC-MS RT = 2.04 min m/z (APCI) 391 [MH⁺](6 min run) | As Example 3 |
| 42a Enantiomer a | 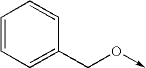 | 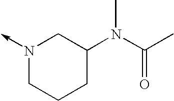 | LC-MS RT = 2.62 min m/z (APCI) 433 [MH⁺](6 min run) | As Example 3 |
| 42b Enantiomer b | 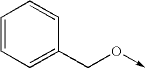 | 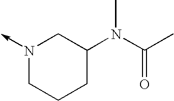 | LC-MS RT = 2.63 min m/z (APCI) 433 [MH⁺](6 min run) | As Example 3 |
| 43 | 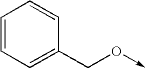 | 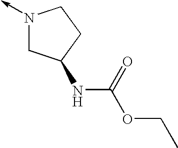 | ¹H NMR (400 MHz, CDCl₃) δ ppm 1.23 (t, 3 H), 1.97-2.06 (m, 1 H), 2.26-2.37 (m, 1 H), 3.42 (dd, 1 H), 3.51-3.63 (m, 2 H), 3.78 (dd, 1 H), 4.08- 4.19 (m, 2 H), 4.38-4.43 (br. m, 1 H), 4.75-4.82 (br. m, 1 H), 5.02 (s, 2 H), 6.01-6.02 (m 2 H), 6.41 (d, 1 H), 7.20 (d, 1 H), | |
| # 7.35-7.43 (m, 5 H), 7.52 (dd, 1 H), 8.04 (s, 1 H). LRMS m/z (ESI) 435 [MH⁺] | As Example 3 | | | |
| 44 | 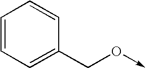 | 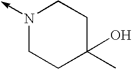 | LC-MS RT = 0.98 min m/z (ESI) 392 [MH⁺](2 min run) | As Example 3 |
| 45 | 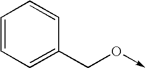 | 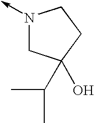 | LC-MS RT = 0.97 min m/z (ESI) 406 [MH⁺](2 min run) | As Example 3 |
| 46 | 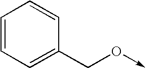 | 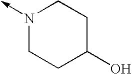 | LC-MS RT = 2.59 min m/z (APCI) 378 [MH⁺](6 min run) | As Example 3 |
| 47 | 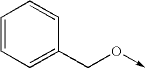 | 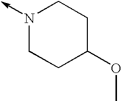 | LC-MS RT = 3.05 min m/z (APCI) 392 [MH⁺](6 min run) | As Example 3 |
| 48 | 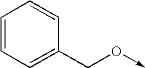 | 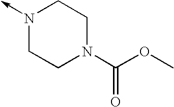 | LC-MS RT = 2.83 min m/z (APCI) 421 [MH⁺](6 min run) | As Example 3 |
| 49 | 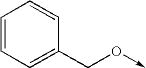 | 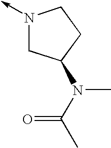 | ¹H NMR (400 MHz, CDCl₃) δ ppm 2.02-2.29 (m, 5 H), 2.88-2.95 (2 × s, 3 H), 3.35-3.51 (m, 2 H), 3.62-3.80 (m, 2 H), 4.55- 5.43 (m, 3 H), 6.00-6.02 (m, 2 H), 6.40-6.43 (m, 1 H), 7.18-7.42 (m, 5 H), 7.48-7.53 (m, 1 H), 8.02- 8.06 (m, 1 H) LRMS m/z (APCI) 419 [MH⁺] | As Example 3 |
| 50 Racemic | 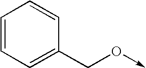 | 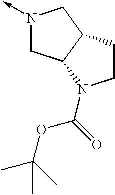 | LC-MS 2.86 min m/z (APCI & ESI) 489 [MH⁺](6 min run) | As Example 3 |
| 51 Racemic | 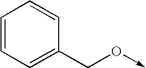 | 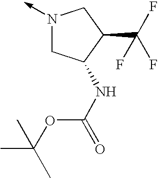 | LC-MS RT = 3.32 min m/z (ESI) 531 [MH⁺](6 min run) | As Example 3 |
| 52 Racemic | 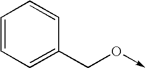 | 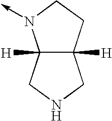 | ¹H NMR (400 MHz CH₃OD) δ ppm 1.70-1.84 (m, 1 H), 2.00-2.14 (m, 1 H), 2.65-2.71 (m, 1 H), 2.80-2.91 (m, 3 H), 2.91- 3.01 (m, 1 H), 3.34-3.46 (m, 2 H), 3.47-3.56 (m, 1 H), 4.22-4.30 (m, 1 H), 5.03 (s, 2 H), 6.04 (d, 1 H), 6.22 (dd, 1 H), 6.61 (d, 1 H), 7.29-7.50 (m, | |
| # 7 H), 7.98 (d, 1 H). LRMS m/z (APCI & ESI) 389 [MH⁺] | As Example 3 followed by Example 8 but in MeOH | | | |
| 53a Enantiomer a | 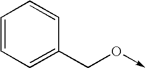 | 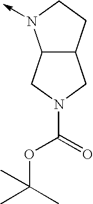 | LC-MS RT = 3.13 min m/z (APCI & ESI) 489 [MH⁺](6 min run) | As Example 3 |
| 53b Enantiomer b | 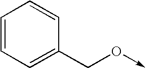 | 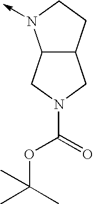 | LC-MS RT = 3.13 min m/z (APCI & ESI) 489 [MH⁺](6 min run) | As Example 3 |
| 54 | 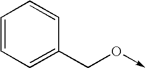 | 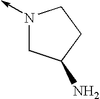 | ¹H NMR (400 MHz, CD₃OD) δ ppm 2.31-2.41 (br. m, 1 H), 2.60-2.64 (br. m, 1 H), 3.41-3.99 (m, 3 H), 4.02-4.18 (m, 1 H), 4.20- 4.25 (m, 1 H), 5.19 (s, 2 H), 6.09 (s, 1 H), 6.30 (m, 1 H), 7.21 (d, 1 H), 7.31-7.45 (m, 5 H), 7.60 (d, 1 H), 8.00 (d, 1 H), 8.20 (s, 1 H) | As Example 3 |
| # followed by Example 13 | | | | |
| 55 | 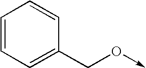 | 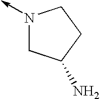 | LC-MS RT= 1.77 min m/z (APCI & ESI) 363 [MH⁺](6 min run) | As Example 3 followed Example 8 but in MeOH then free base obtained |
| 56 | 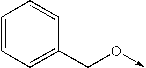 | 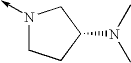 | ¹H NMR (400 MHz, CD₃OD) δ ppm 1.83-1.99 (m, 1 H), 2.32 (m, 1 H), 2.32 (s, 6 H), 2.90-3.01 (m, 1 H), 3.28-3.32 (m, 1 H), 3.40- 3.52 (m, 1 H), 3.62-3.69 (m, 1 H), 3.80-3.83 (m, 1 H), 5.19 (s, 2 H), 6.09 (s, 1 H), 6.22 (d, 1 H), 6.59 (d, 1 H), 7.35-7.60 (m, 7 H), 8.02 (s, 1 H) | |
| # LRMS m/z (APCI & ESI) 391 [MH⁺] | As Example 3 | | | |
| 57 | 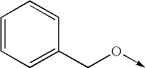 | 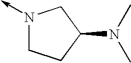 | ¹H NMR (400 MHz, CD₃OD) δ ppm 1.83-1.99 (m, 1 H), 2.32 (m, 1 H), 2.32 (s, 6 H), 2.90-3.01 (m, 1 H), 3.28-3.32 (m, 1 H), 3.40- 3.52 (m, 1 H), 3.62-3.69 (m, 1 H), 3.80-3.83 (m, 1 H), 5.19 (s, 2 H), 6.09 (s, 1 H), 6.22 (d, 1 H), 6.59 (d, 1 H), 7.35-7.60 (m, 7 H), 8.02 (s, 1 H) | |
| # LRMS m/z (APCI & ESI) 391 [MH⁺] | As Example 3 | | | |
| 58 | 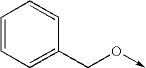 | 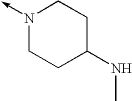 | ¹H NMR (400 MHz, CD₃OD) δ ppm 1.80-1.91 (br. m, 2 H), 2.25-2.31 (br. m, 2 H), 2.78 (s, 3 H), 3.40-3.82 (m, 3 H), 4.32- 4.42 (m, 2 H), 5.19 (s, 2 H), 6.12-6.18 (br. m, 1 H), 6.36-6.39 (br. m, 1 H), 7.31-7.47 (m, 5 H), 7.47-7.70 (m, 1 H), 8.13 (br. d, 1 H), 8.20 (s, 1 H) | |
| # LRMS m/z (APCI & ESI) 391 [MH⁺] | As Example 3 followed by Example 13 | | | |
| 59 | 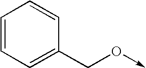 | 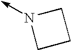 | ¹H NMR (400 MHz, d₆- DMSO) δ ppm 2.22-2.38 (m, 2 H), 3.91-4.05 (m, 4 H), 5.19 (s, 2 H), 5.95 (s, 1 H), 6.10 (d, 1 H), 6.41 (d, 1 H) 7.51-7.62 (m, 7 H), 7.91 (s, 1 H) | As Example 3 |
| 60 | 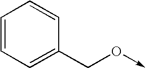 | 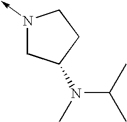 | ¹H NMR (400 MHz, d₆- DMSO) δ ppm 0.95 (d, 6 H), 1.71-1.82 (m, 1 H), 2.05-2.21 (m, 4 H), 2.87- 3.38 (m, 4 H), 3.57-3.60 (m, 1 H), 3.61-3.70 (m, 1 H), 5.18 (s, 2 H), 5.91 (s, 1 H), 6.20 (d, 1 H), 6.51 (d, 1 H), 7.35-7.59 (m, 7 H), 7.98 (s, 1 H) | As Example 3 |
| 61 | 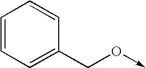 | 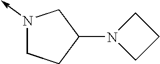 | ¹H NMR (400 MHz, CD₃OD) δ ppm 1.85-1.90 (m, 1 H), 2.01-2.19 (m, 3 H), 3.18-3.20 (m, 1 H), 3.30-3.39 (m, 5 H), 3.43- 3.61 (m, 3 H), 5.18 (s, 2 H), 6.21 (s, 1 H), 6.51 (d, 1 H), 6.57 (d, 1 H), 7.33-7.57 (m, 5 H), 7.98 (s, 1 H) LRMS m/z (APCI & ESI) 403 [MH⁺] | As Example 3 |
| 62a Enantiomer a | 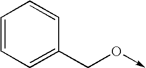 | 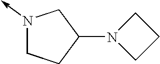 | ¹H NMR (400 MHz, CDCl₃) δ ppm 1.82-1.89 (m, 1 H), 1.93-1.97 (m, 1 H), 2.04-2.11 (m, 2 H), 3.00-3.05 (m, 1 H), 3.24 (t, 4 H), 3.30-3.33 (m, 1 H), 3.43-3.49 (m, 2 H), 3.52-3.58 (m, 1 H), 5.03 (s, 2 H), 6.02 (d, 2 H), 6.38 (d, 1 H), 7.18 (d, 1 H), 7.34-7.41 (m, 5 H), | |
| # 7.50 (d, 1 H), 8.03 (s, 1 H) LRMS m/z (APCI & ESI) 403 [MH⁺] | As Example 3 | | | |
| 62b Enantiomer b | 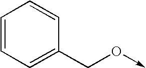 | 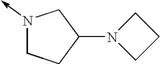 | ¹H NMR (400 MHz, CDCl₃) δ ppm 1.82-1.89 (m, 1 H), 1.93-1.97 (m, 1 H), 2.04-2.11 (m, 2 H), 3.00-3.05 (m, 1 H), 3.24 (t, 4 H), 3.30-3.33 (m, 1 H), 3.43-3.49 (m, 2 H), 3.52-3.58 (m, 1 H), 5.03 (s, 2 H), 6.02 (d, 2 H), 6.38 (d, 1 H), 7.18 (d, 1 H), 7.34-7.41 (m, 5 H), | |
| # 7.50 (d, 1 H), 8.03 (s, 1 H) LRMS m/z (APCI & ESI) 403 [MH⁺] | As Example 3 | | | |
| 63 | 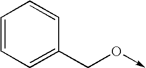 | 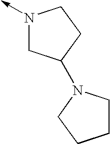 | ¹H NMR (400 MHz, CD₃OD) 1.82-1.86 (m, 4 H), 1.95-2.04 (m, 1 H), 2.24-2.31 (m, 1 H), 2.63- 2.68 (m, 4 H), 2.93-3.02 (m, 1 H), 3.39-3.46 (m, 1 H), 3.63-3.68 (m, 1 H), 3.73-3.78 (m, 1 H), 5.12 (s, 2 H), 6.05 (s, 1 H), 6.22 (dd, 1 H), 6.56 (d, 1 H), 7.29-7.50 (m, 8 H), 7.97 (s, 1 H) | |
| # LRMS m/z (APCI & ESI) 417 [MH⁺] | As Example 3 | | | |
| 64 racemic | 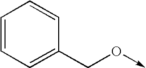 | 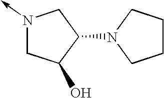 | ¹H NMR (400 MHz, CD₃OD) δ ppm 1.80- 1.88 (m, 4 H), 2.70-2.80 (m, 4 H), 2.92 (dd, 1 H), 3.35 (dd, 1 H), 3.51 (dd, 1 H), 3.80-3.88 (m, 2 H), 4.43 (dd, 1 H), 5.16 (s, 2 H), 6.08 (d, 1 H), 6.26 (dd, 1 H), 6.60 (d, 1 H), 7.33-7.54 (m, 7 H), 8.01 (d, 1 H) LRMS m/z (APCI & ESI) | |
| # 433 [MH⁺] | As Example 3 | | | |
| 65 racemic | 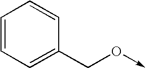 | 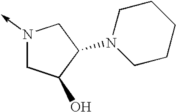 | ¹H NMR (400 MHz, CD₃OD) δ ppm 1.48-1.53 (m, 2 H), 1.61-1.67 (m, 4 H), 2.54-2.59 (m, 2 H), 2.72-2.77 (m, 2 H), 3.01 (dd, 1 H), 3.32 (dd, 1 H), 3.44 (dd, 1 H), 3.81-3.89 (m, 2 H), 4.47 (dd, 1 H), 5.16 (s, 2 H), 6.08 (d, 1 H), 6.26 (dd, 1 H), 6.60 (d, 1 H), 7.33-7.55 (m, | |
| # 7 H), 8.02 (d, 1 H) LRMS m/z (APCI & ESI) 447 [MH⁺] | As Example 3 | | | |
| 66 racemic | 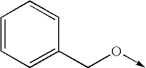 | 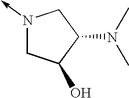 | ¹H NMR (400 MHz, CD₃OD) δ ppm 2.40 (s, 6 H), 2.92 (dd, 1 H), 3.34 (dd, 1 H), 3.44 (dd, 1 H), 3.80-3.89 (m, 2 H), 4.42 (dd, 1 H), 5.15 (s, 2 H), 6.08 (d, 1 H), 6.28 (dd, 1 H), 6.60 (d, 1 H), 7.33- 7.55 (m, 7 H), 8.02 (d, 1 H) LRMS m/z (APCI & ESI) 407 [MH⁺] | As Example 3 |
| 67 Racemic | 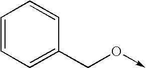 | 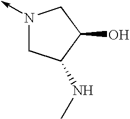 | LC-MS RT = 1.93 min m/z (APCI & ESI) 393 [MH⁺](6 min run) | As Example 3 |
| 68 S, S | 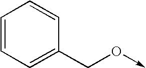 | 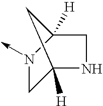 | LC-MS RT = 1.98 min m/z (APCI & ESI) 375 [MH⁺](6 min run) | Example 3 but with DIPEA followed by Example 8 |
| 69 | 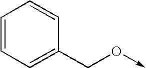 | 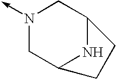 | LC-MS RT = 2.06 min m/z (APCI & ESI) 389 [MH⁺](6 min run) | Example 3 but with DIPEA followed by Example 8 |
| 70 | 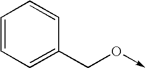 | 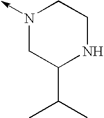 | LC-MS RT = 2.15 min m/z (APCI & ESI) 405 [MH⁺](6 min run) | Example 3 but with DIPEA followed by Example 8 |
| 71 | 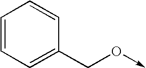 | 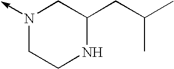 | LC-MS RT = 2.26 min m/z (APCI & ESI) 419 [MH⁺](6 min run) | Example 3 but with DIPEA followed by Example 8 |
| 72 | 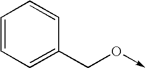 | 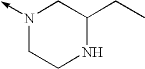 | LC-MS RT = 2.08 min m/z (APCI & ESI) 391 [MH⁺](6 min run) | Example 3 but with DIPEA followed by Example 8 |
| 73 | 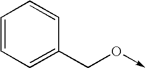 | 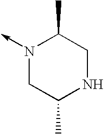 | LC-MS RT = 2.10 min m/z (APCI & ESI) 391 [MH⁺](6 min run) | Example 3 but with DIPEA followed by Example 8 |
| 74 Racemic | 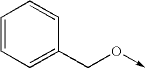 | 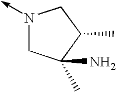 | LC-MS RT = 2.00 min m/z (APCI & ESI) 391 [MH⁺](6 min run) | Example 3 but with DIPEA followed by Example 8 |
| 75 Racemic | 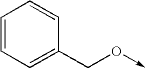 | 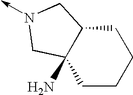 | LC-MS RT = 2.12 min m/z (APCI & ESI) 417 [MH⁺](6 min run) | Example 3 but with DIPEA followed by Example 8 |
| 76 | 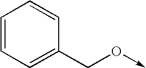 | 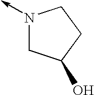 | LC-MS RT = 2.65 min m/z (APCI & ESI) 364 [MH⁺](6 min basic run) | As Example 3 but with Et₃N |
| 77 S | 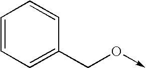 | 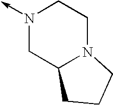 | LC-MS RT = 3.14 min m/z (APCI & ESI) 403 [MH⁺](6 min basic run) | As Example 3 but with Et₃N |
| 78 | 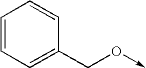 | 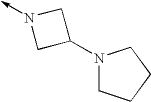 | LC-MS RT = 3.01 min m/z (APCI & ESI) 403 [MH⁺](6 min basic run) | As Example 3 but with Et₃N |
| 79 | 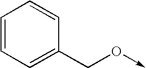 | 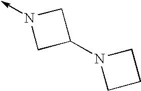 | LC-MS RT = 0.18 min m/z (APCI & ESI) 389 [MH⁺](2 min run) | As Example 3 but with Et₃N |
| 80 Racemic | 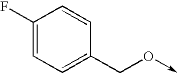 | 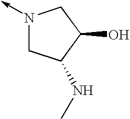 | LC-MS RT = 2.02 min m/z (APCI & ESI) 411 [MH⁺](6 min run) | As Example 3 |
| 81 | 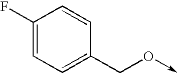 | 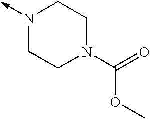 | ¹H NMR (400 MHz, CDCl₃) δ ppm 3.60 (s, 8 H), 3.78 (s, 3 H), 5.00 (s, 2 H), 6.02 (s, d, 2 H), 6.70 (d, 1 H), 7.10 (t, 1 H), 7.19 (dd, 1 H), 7.40 (dd, | |
| # 1 H), 7.57 (dd, 1 H), 8.10 (s, 1 H). LRMS m/z (APCI) 477 [M + K]⁺ | As Example 3 | | | |
| 82a Enantiomer a | 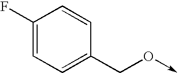 | 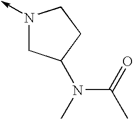 | ¹H NMR (400 MHz, CDCl₃) δ ppm 2.12-2.21 (2 × s, 3 H), 2.21-2.30 (m, 2 H), 2.88-2.93 (2 × s, 3 H), 3.39-3.50 (m, 2 H), | |
| # 3.63-3.79 (m, 2 H), 4.57- 5.43 (m, 3 H), 6.01 (s, d, 2 H), 6.41 (d, 1 H), 7.09 (t, 2 H), 7.19 (dd, 1 H), 7.38 (dd, 2 H), 7.52 (dd, 1 H), 8.10 (s, 1 H). LRMS m/z (APCI) 437 [MH⁺] | | | | |
| # [α]_{D}²⁵ +44.72, (2.65 mg/mL, MeOH) | As Example 3 | | | |
| 82b Enantiomer b | 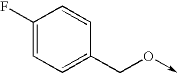 | 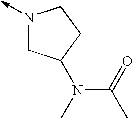 | ¹H NMR (400 MHz, CDCl₃) δ ppm 2.12-2.21 (2 × s, 3 H), 2.21-2.30 (m, 2 H), 2.88-2.93 (2 × s, 3 H), 3.39-3.50 (m, 2 H), | |
| # 3.63-3.79 (m, 2 H), 4.57- 5.43 (m, 3 H), 6.01 (s, d, 2 H), 6.41 (d, 1 H), 7.09 (t, 2 H), 7.19 (dd, 1 H), 7.38 (dd, 2 H), 7.52 (dd, 1 H), 8.10 (s, 1 H). LRMS m/z (APCI) 437 [MH⁺] | | | | |
| # α]_{D}²⁵ +44.72, (2.65 mg/mL, MeOH) | As Example 3 | | | |
| 83 | 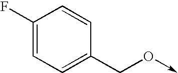 | 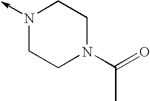 | LC-MS RT = 2.63 m/z (APCI) 423 [MH⁺](6 min run) | As Example 3 |
| 84 | 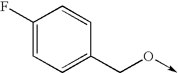 | 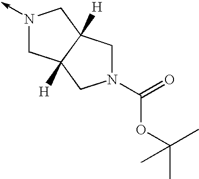 | LC-MS RT = 2.80 m/z (APCI) 407 [MH⁺ (−BOC)](6 min run) | As Example 3 |
| 85 S, S | 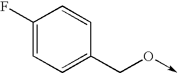 | 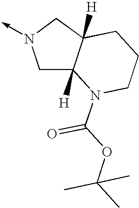 | LC-MS RT = 3.09 m/z (APCI & ESI) 521 [MH⁺](6 min run) | As Example 3 |
| 86 R, R | 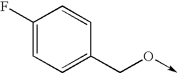 | 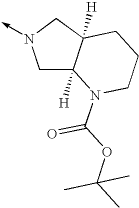 | LC-MS RT = 3.02 m/z (APCI & ESI) 521 [MH⁺](6 min run) | As Example 3 |
| 87 | 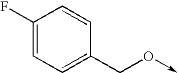 | 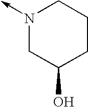 | LC-MS RT = 2.42 m/z (APCI & ESI) 396 [MH⁺](6 min run) | As Example 3 |
| 88 | 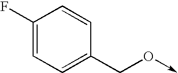 | 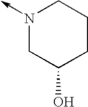 | LC-MS RT = 245 m/z (APCI & ESI) 396 [MH⁺](6 min run) | As Example 3 |
| 89a Enantiomer a | 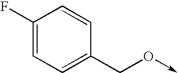 | 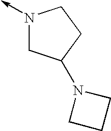 | ¹H NMR (400 MHz, CD₃OD) δ ppm 1.82-1.95 (m, 1 H), 2.01-2.19 (m, 3 H), 3.19-3.21 (m, 1 H), 3.27-3.39 (m, 5 H), 3.41- 3.60 (m, 3 H), 5.18 (s, | |
| # 2 H), 6.09 (s, 1 H), 6.21 (d, 1 H), 6.57 (d, 1 H), 7.17 (t, 2 H), 7.43-7.57 (m, 4 H), 7.98 (s, 1 H) LRMS m/z (APCI & ESI) 421 [MH⁺] | As Example 3 | | | |
| 89b Enantiomer b | 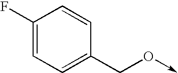 | 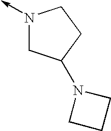 | ¹H NMR (400 MHz, CD₃OD) δ ppm 1.82-1.95 (m, 1 H), 2.01-2.19 (m, 3 H), 3.19-3.21 (m, 1 H), 3.27-3.39 (m, 5 H), 3.41- 3.60 (m, 3 H), 5.18 (s, | |
| # 2 H), 6.09 (s, 1 H), 6.21 (d, 1 H), 6.57 (d, 1 H), 7.17 (t, 2 H), 7.43-7.57 (m, 4 H), 7.98 (s, 1 H) LRMS m/z (APCI & ESI) 421 [MH⁺] | As Example 3 | | | |
| 90 | 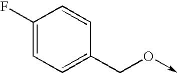 | 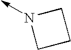 | LC-MS RT = 2.20 m/z (APCI & ESI) 352 [MH⁺](6 min run) | As Example 3 |
| 91 | 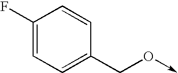 | 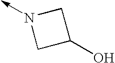 | LC-MS RT = 2.17 m/z (APCI & ESI) 368 [MH⁺](6 min run) | As Example 3 |
| 92 | 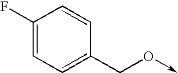 | 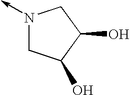 | LC-MS RT = 2.34 m/z (ESI) 795 [M₂H]⁺(HPLC) | As Example 3 |
| 93 | 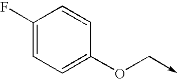 | 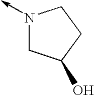 | LC-MS RT = 2.68 m/z (APCI & ESI) 382 [MH⁺](6 min basic run) | As Example 3 but with Et₃N |
| 94 S | 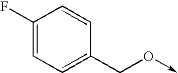 | 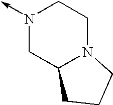 | LC-MS RT = 3.16 m/z (APCI & ESI) 421 [MH⁺](6 min basic run) | As Example 3 but with Et₃N |
| 95 | 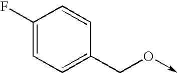 | 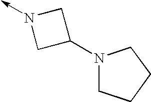 | LC-MS RT = 3.03 min m/z (APCI & ESI) 421 [MH⁺](6 min basic run) | As example 3 but with Et₃N |
| 96 | 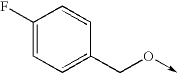 | 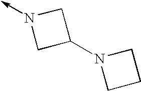 | LC-MS RT = 0.57 min m/z (APCI & ESI) 407 [MH⁺](2 min run) | As Example 3 but with Et₃N |
| 97 Racemic | 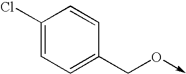 | 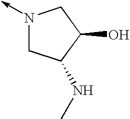 | LC-MS RT = 2.14 m/z (APCI & ESI) 427/429 [MH⁺](6 min run) | As Example 3 |
| 98 S, S enatiomer | 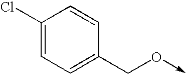 | 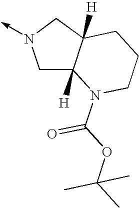 | LC-MS RT = 3.24 m/z (APCI & ESI) 537 [MH⁺](6 min run) | As Example 3 |
| 99 R, R enatiomer | 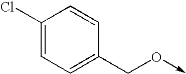 | 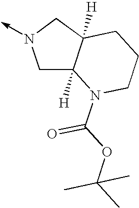 | LC-MS RT = 3.25 m/z (APCI & ESI) 537 [MH⁺](6 min run) | As Example 3 |
| 100a Enantiomer a | 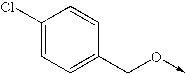 | 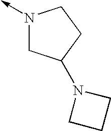 | ¹H NMR (400 MHz, CD₃OD) δ ppm 1.82-1.95 (m, 1 H), 2.01-2.19 (m, 3 H), 3.10-3.20 (m, 1 H), 3.27-3.39 (m, 5 H), 3.41- 3.60 (m, 3 H), 5.18 (s, | |
| # 2 H), 6.09 (s, 1 H), 6.21 (d, 1 H), 6.57 (d, 1 H), 7.38-7.57 (m, 6 H), 7.98 (s, 1 H) LRMS m/z (APCI & ESI) 437 [MH⁺] | As Example 3 | | | |
| 100b Enantiomer b | 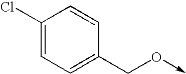 | 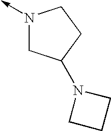 | ¹H NMR (400 MHz, CD₃OD) δ ppm 1.82-1.95 (m, 1 H), 2.01-2.19 (m, 3 H), 3.13-3.20 (m, 1 H), 3.27-3.39 (m, 5 H), 3.41- 3.60 (m, 3 H), 5.18 (s, | |
| # 2 H), 6.09 (s, 1 H), 6.21 (d, 1 H), 6.57 (d, 1 H), 7.38-7.57 (m, 6 H), 7.98 (s, 1 H) | As Example 3 | | | |
| 101 | 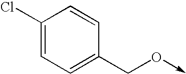 | 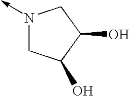 | LC-MS RT = 2.28 m/z (APCI & ESI) 414 [MH⁺](6 min run) | As Example 3 |
| 102 Racemic | 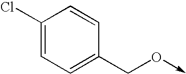 | 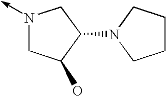 | ¹H NMR (400 MHz, CD₃OD) δ ppm 1.82-1.86 (m, 4 H), 2.68-2.78 (m, 4 H), 2.91 (dd, 1 H), 3.35 (dd, 1 H), 3.51 (dd, 1 H), 3.80-3.89 (m, 2 H), 4.43 | |
| # (dd, 1 H), 5.15 (s, 2 H), 6.08 (d, 1 H), 6.26 (dd, 1 H), 6.61 (d, 1 H), 7.41- 7.55 (m, 6 H), 8.02 (d, 1 H) LRMS m/z (APCI & ESI) 467/469 [MH⁺] | As Example 3 | | | |
| 103 Racemic | 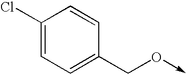 | 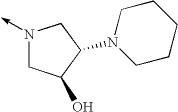 | ¹H NMR (400 MHz, CD₃OD) δ ppm 1.47-1.53 (m, 2 H), 1.61-1.67 (m, 4 H), 2.54-2.59 (m, 2 H), 2.71-2.77 (m, 2 H), 3.01 (dd, 1 H), 3.33 (dd, 1 H), | |
| # 3.43 (dd, 1 H), 3.81-3.89 (m, 2 H), 4.47 (dd, 1 H), 5.14 (s, 2 H), 6.07 (d, 1 H), 6.25 (dd, 1 H), 6.60 (d, 1 H), 7.40-7.54 (m, 6 H), 8.02 (d, 1 H) LRMS m/z (APCI & ESI) 481/483 [MH⁺] | As Example 3 | | | |
| 104 Racemic | 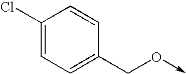 | 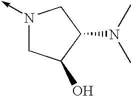 | ¹H NMR (400 MHz, CD₃OD) δ ppm 2.39 (s, 6 H), 2.91 (dd, 1 H), 3.33 (dd, 1 H), 3.44 (dd, 1 H), 3.80-3.89 (m, 2 H), 4.42 (dd, 1 H), 5.14 (s, 2 H), | |
| # 6.07 (d, 1 H), 6.26 (dd, 1 H), 6.60 (d, 1 H), 7.40- 7.55 (m, 6 H), 8.02 (d, 1 H) LRMS m/z (APCI & ESI) 441/443 [MH⁺] | As Example 3 | | | |
| 105 | 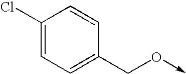 | 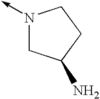 | ¹H NMR (400 MHz, CDCl₃) δ ppm 1.60-1.84 (m, 1 H + NH2 under H2O), 2.20-2.26 (m, 1 H), 3.22 (dd, 1 H), 3.48-3.53 (m, 1 H), 3.61-3.78 (m, 3 H), 5.00 (s, 2 H), 6.00-6.02 (m, | |
| # 2 H), 6.04 (dd, 1 H), 7.20 (d, 1 H), 7.38 (q, 4 H), 7.46 (dd, 1 H), 8.03 (d, 1 H). | As Example 3, followed by Example 13 (re. de- Bocylation) | | | |
| 106 | 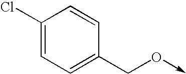 | 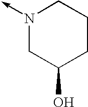 | LC-MS RT = 3.13 min m/z (ESI) 412 [MH⁺](HPLC) | Example 3 but with Et₃N |
| 107 | 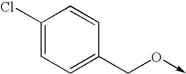 | 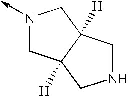 | LC-MS RT = 2.93 min m/z (APCI & ESI) 423/425 [MH⁺](6 min acidic run) | As Example 3 followed by Example 13 |
| 108 S, S | 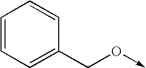 | 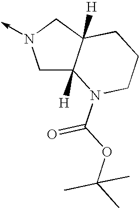 | LC-MS RT = 2.99 min m/z (APCI & ESI) 503 [MH⁺](6 min run) | As Example 11 |
| 109 R, R | 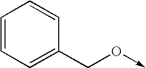 | 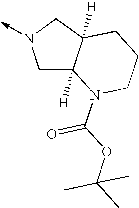 | LC-MS RT = 3.01 min m/z (APCI & ESI) 503 [MH⁺](6 min run) | As Example 11 |
| 110 | 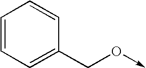 | 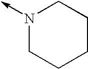 | ¹H NMR (400 MHz, d₆- DMSO) δ ppm 1.45-1.68 (m, 6 H), 3.59- 3.64 (m, 4 H), 5.19 (s, 2 H), 5.98 (s, 1 H), 6.10 (d, 1 H), 6.91 (d, 1 H) 7.35-7.59 (m, 7 H), 8.10 | |
| # (s, 1 H) | As Example 11 but at 110° C. | | | |
| 111 | 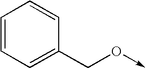 | 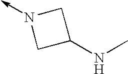 | LC-MS RT = 2.65 min m/z (APCI & ESI) 363 [MH⁺](6 min basic run) | As Example 11 followed by Example 13 |
| 112 | 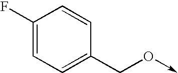 | 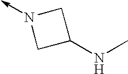 | LC-MS RT = 2.69 min m/z (APCI & ESI) 381 [MH⁺](6 min basic run) | As Example 11 followed by Example 13 |
| 113 | 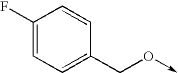 | 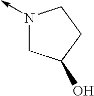 | LC-MS RT = 2.12 min m/z (APCI & ESI) 382 [MH⁺](6 min run) | As Example 11 but in n-butanol |
| 114 | 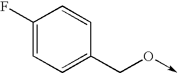 | 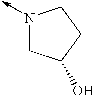 | LC-MS RT = 2.12 min m/z (APCI & ESI) 382 [MH⁺](6 min run) | As Example 11 |
| 115 | 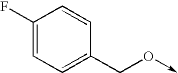 | 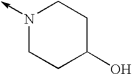 | LC-MS RT = 2.39 min m/z (APCI & ESI) 396 [MH⁺](6 min run) | As Example 11 |
| 116 | 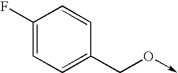 | 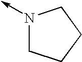 | LC-MS RT = 2.22 min m/z (APCI & ESI) 366 [MH⁺](6 min run) | As Example 11 |
| 117 | 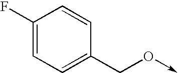 | 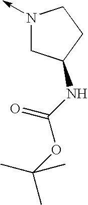 | LC-MS RT = 2.65 min m/z (APCI & ESI) 425 [MH⁺](6 min run) | As Example 11 |
| 118 | 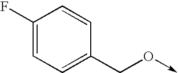 | 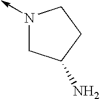 | ¹H NMR (400 MHz CH₃OD) δ ppm 1.93 (br. s, 1 H), 2.29 (br. s, 1 H), 3.44-3.85 (m, 5 H), 5.13 (s, 2 H), 6.09 (d, 1 H), 6.24 (d, 1 H), 6.58 (d, | |
| # 1 H), 7.15 (t, 2 H), 7.37- 7.58 (m, 4 H), 8.00 (s, 1 H). LRMS m/z (APCI & ESI) 381 [MH⁺] | As Example 3 but with Et₃N followed by Example 8 but MeOH | | | |
| 119 | 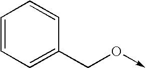 | 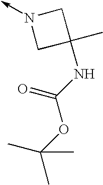 | LC-MS RT = 1.39 min m/z (APCI & ESI) 463 [MH⁺](2 min run) | As Example 3 but with Et₃N at 120° C. |
| 120 | 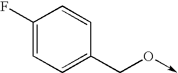 | 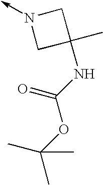 | LC-MS RT = 1.41 min m/z (APCI & ESI) 481 [MH⁺](2 min run) | As Example 3 but with Et₃N at 120° C. |
| 121 | 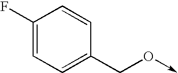 | 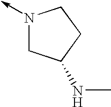 | LC-MS RT = 0.95 min m/z (APCI & ESI) 395 [MH⁺](2 min run) | As Example 3 but with Et₃N at 120° C. followed by Example 13 |
| 122 | 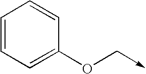 | 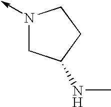 | LC-MS RT = 0.93 min m/z (APCI & ESI) 377 [MH⁺](2 min run) | As Example 3 but with Et₃N at 120° C. followed by Example 13 |
| 123a enriched <89% ee | 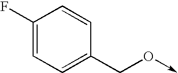 | 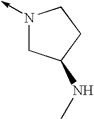 | LC-MS RT = 1.86 min m/z (APCI & ESI) 395 [MH⁺](6 min acidic run) | As Example 3 but in IPA at 120° C. |
| # followed by Example 13 | | | | |
| 123b | 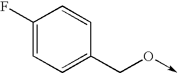 | 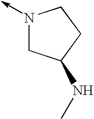 | ¹HNMR (400 MHz CD₃OD) δ ppm 2.35-2.71 (m, 2 H), 2.82 (s, 3 H), 3.82-3.90 (m, 1 H), 3.97- 4.07 (m, 2 H), 4.11-4.20 (m, 2 H), 5.20 (s, 2 H), | |
| # 6.23 (d, 1 H), 6.47 (dd, 1 H), 7.15 (t, 2 H), 7.30 (d, 1 H), 7.50 (dd, 2 H), 7.76 (d, 1 H), 8.18 (dd, 1 H), 8.23 (s, 1 H) LRMS m/z (APCI & ESI) 395 [MH⁺] | As Example 3 but in IPA | | | |
| # at 110° C. followed by Example 13 | | | | |
| 124 | 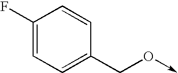 | 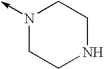 | LC-MS RT = 2.04 min m/z (APCI & ESI) 381 [MH⁺](6 min run) | As Example 3 but in IPA at 100° C. followed by Example 8 |

Examples 125-143 were prepared according to the methods described above for examples 8, 13 and 17, starting from the appropriate Boc protected compound¹.
1. The appropriate Boc protected starting materials are listed in table below.

| | |
|---|---|
| | I |
| 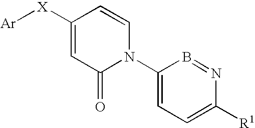 | |

| | | | | | Preparation & |
|---|---|---|---|---|---|
| Example | Ar—X | B | R¹ | Analytical Data | Starting Material |
| 125 | 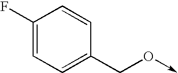 | CH | 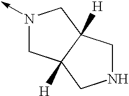 | ¹H NMR (400 MHz, CD₃OD) δ ppm 3.37-3.46 (m, 4 H), 3.63-3.67 (m, 2 H), 3.69 (dd, 2 H), 3.98 (dd, 2 H), 5.17 (s, 2 H), 6.10 (d, 1 H), 6.32 (dd, 1 H), 7.15 (t, | |
| # 2 H), 7.23 (d, 1 H), 7.46 (dd, 2 H), 7.59 (d, 1 H), 8.09 (dd, 1 H), 8.18 (d, 1 H). LRMS m/z (APCI) 407 [MH⁺] | As Example 8 from Example 84 | | | | |
| 126 | 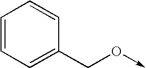 | CH | 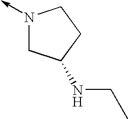 | ⁺NMR (400 MHz, CDCl₃) δ ppm 1.17 (t, 3 H), 1.86-1.94 (m, 1 H), 2.20-2.29 (m, 1 H), 2.78 (q, 2 H), 3.31 (dd, 1 H), 3.43-3.57 (m, 2 H), 3.60-3.67 (m | |
| # 1 H), 3.75 (dd, 1 H), 5.02 (s, 2 H), 6.01- 6.02 (m 2 H), 6.41 (d, 1 H), 7.19 (d, 1 H), 7.32-7.45 (m, 5 H), 7.45 (dd, 1 H), 8.02 (s, 1 H). LRMS m/z (ESII) 391 [MH⁺] | As Example 8 from Example 23b | | | | |
| 127 | 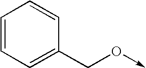 | N | 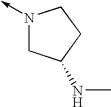 | LC-MS RT = 2.48 min m/z (APCI & ESI) 378 [MH⁺](6 min run) | As Example 8 from Example 156 |
| 128 | 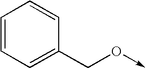 | N | 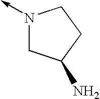 | LC-MS RT = 2.17 min m/z (APCI & ESI) 364 [MH⁺](6 min run) | As Example 8 from Example 155 |
| 129 | 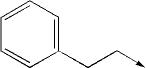 | CH | 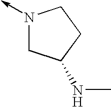 | ¹H NMR (400 MHz CH₃OD) δ ppm 1.87- 1.97 (m, 1 H), 2.21- 2.31 (m, 1 H), 2.42 (s, 3 H), 2.81-2.89 (m, 2 H), 2.91-3.00 (m, 2 H), 3.32-3.37 (m, 1 H), 3.38-3.42 (m, | |
| # 1 H), 3.43-3.51 (m, 1 H), 3.66-3.72 (m, 1 H), 3.85 (s, 2 H), 6.31 (dd, 1 H), 6.40 (s, 1 H), 6.58 (d, 1 H), 7.18-7.36 (m, 5 H), 7.42 (d, 1 H), 7.51 (dd, 1 H), 7.99 (d, 1 H). LRMS m/z (APCI & | | | | | |
| # ESI) 375 [MH⁺] | Example 8 but in MeOH from Example 32 | | | | |
| 130 S, S | 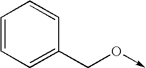 | CH | 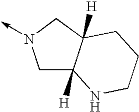 | ¹H NMR (400 MHz CH₃OD) δ ppm 1.44- 1.53 (m, 1 H), 1.58- 1.71 (m, 1 H), 1.75- 1.82 (m, 2 H), 2.36- 2.47 (m, 1 H), 2.57- 2.67 (m, 1 H), 2.88- 2.95 (m, 1 H), 3.39- | |
| # 3.60 (m, 5 H), 5.12 (s, 2 H), 6.04 (d, 1 H), 6.22 (dd, 1 H), 6.54 (d, 1 H), 7.29-7.50 (m, 7 H), 7.94 (d, 1 H). LRMS m/z (APCI & ESI) 403 [MH⁺] | Example 8 but in MeOH from Example 108 | | | | |
| 131 R, R | 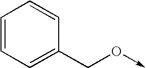 | CH | 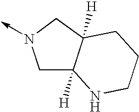 | LC-MS RT = 1.88 min m/z (APCI & ESI) 403 [MH⁺](6 min run) | Example 8 but in MeOH from Example 109 |
| 132 Racemic | 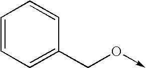 | CH | 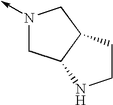 | LC-MS RT = 1.89 min m/z (APCI & ESI) 364 [MH⁺](6 min run) | Example 8 but in MeOH from Example 50 |
| 133 S, S | 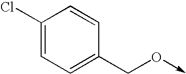 | CH | 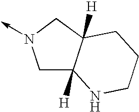 | LC-MS RT = 1.95 min m/z (APCI & ESI) 437 [MH⁺](6 min run) | Example 8 but in MeOH from Example 98 |
| 134 R, R | 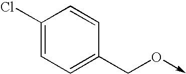 | CH | 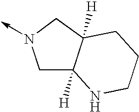 | LC-MS RT = 1.95 min m/z (APCI & ESI) 437 [MH⁺](6 min run) | Example 8 but in MeOH from Example 99 |
| 135 S, S | 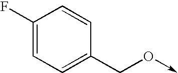 | CH | 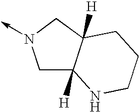 | LC-MS RT = 1.86 min m/z (APCI & ESI) 421 [MH⁺](6 min run) | Example 8 but in MeOH from Example 85 |
| 136 R, R | 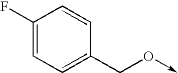 | CH | 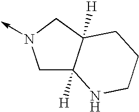 | LC-MS RT = 2.05 min m/z (APCI & ESI) 421 [MH⁺](6 min run) | Example 8 but in MeOH from Example 86 |
| 137 Racemic | 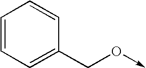 | CH | 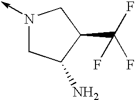 | LC-MS RT = 2.20 min m/z (APCI & ESI) 431 [MH⁺](6 min run) | Example 8 but in MeOH from Example 51 |
| 138a Enantiomer a | 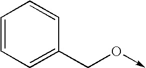 | CH | 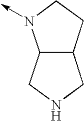 | LC-MS RT = 1.86 min m/z (APCI & ESI) 389 [MH⁺](6 min run) | Example 8 but in MeOH from Example 53a |
| 138b Enantiomer b | 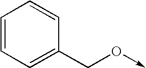 | CH | 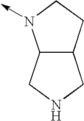 | LC-MS RT = 1.87 min m/z (APCI & ESI) 389 [MH⁺](6 min run) | Example 8 but in MeOH from Example 53b |
| 139 | 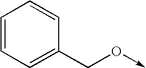 | CH | 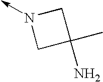 | LC-MS RT = 0.96 min m/z (APCI & ESI) 363 [MH⁺](2 min run) | As Example 13 from Example 119 |
| 140 | 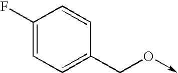 | CH | 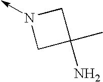 | LC-MS RT = 1.00 min m/z (APCI & ESI) 381 [MH⁺](2 min run) | As Example 13 from Example 120 |
| 141 | 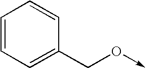 | CH | 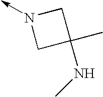 | LC-MS RT = 0.97 min m/z (APCI & ESI) 377 [MH⁺](2 min run) | As Example 13 from Preparation 22 |
| 142 | 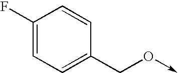 | CH | 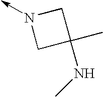 | LC-MS RT = 1.00 min m/z (APCI & ESI) 395 [MH⁺](2 min run) | As Example 13 from Preparation 23 |
| 143 | 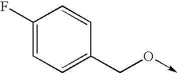 | CH | 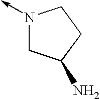 | ¹H NMR (400 MHz, CD₃OD) δ ppm 3.40-4.15 (m, 7 H), 5.21 (s, 2 H), 6.21 (s, 1 H), 6.41 (d, 1 H), 7.12 (t, 2 H), 7.24 (d, 1 H), 7.50 (m, 2 H), 7.69 (d, 1 H), 8.15 (d, | |
| # 1 H), 8.23 (s, 1 H) LRMS m/z (APCI & ESI) 381 [MH⁺] | As Example 13 from Example 117 | | | | |

Examples 144-154 were prepared according to the method described above for Example 10, starting from the appropriate 4-hydroxypyridinone¹ and the appropriate benzyl bromide².
1. 4-Hydroxy-6′-[(3R)-3-hydroxypyrrolidin-1-yl]-2H-1,3′-bipyridin-2-one from Preparation 4 and tert-Butyl[(3S)-1-(4-hydroxy-2-oxo-2H-1,3′-bipyridin-6′-yl)pyrrolidin-3-yl]methylcarbamate from Preparation 24

2. Benzyl bromides were commercially available.

| | |
|---|---|
| | I |
| 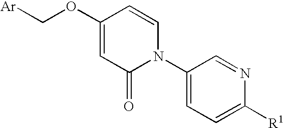 | |

| Example | Ar | R¹ | Analytical Data | Preparation |
|---|---|---|---|---|
| 144 | 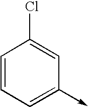 | 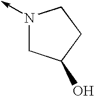 | LC-MS RT = 2.23 min m/z (APCI & ESI) 398/400 [MH⁺](6 min run) | 50° C. |
| 145 | 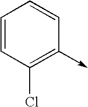 | 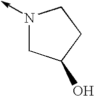 | LC-MS RT = 2.18 min m/z (APCI & ESI) 398/400 [MH⁺](6 min run) | 50° C. |
| 146 | 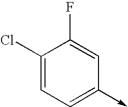 | 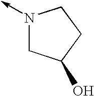 | LC-MS RT = 2.27 min m/z (APCI & ESI) 416/418 [MH⁺](6 min run) | 50° C. |
| 147 | 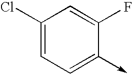 | 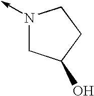 | LC-MS RT = 2.26 min m/z (APCI & ESI) 416/418 [MH⁺](6 min run) | 50° C. |
| 148 | 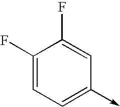 | 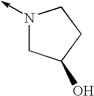 | LC-MS RT = 2.16 min m/z (APCI & ESI) 400 [MH⁺](6 min run) | 50° C. |
| 149 | 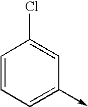 | 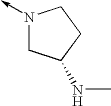 | LC-MS RT = 2.09 min m/z (APCI & ESI) 411/413 [MH⁺](6 min run) | RT followed by Example 8 |
| 150 | 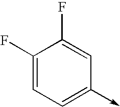 | 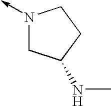 | LC-MS RT = 2.03 min m/z (APCI & ESI) 413 [MH⁺](6 min run) | RT followed by Example 8 |
| 151 | 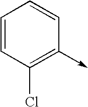 | 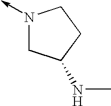 | LC-MS RT = 2.04 min m/z (APCI & ESI) 411/413 [MH⁺](6 min run) | RT followed by Example 8 |
| 152 | 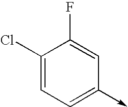 | 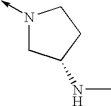 | LC-MS RT = 2.14 min m/z (APCI & ESI) 429/431 [MH⁺](6 min run) | RT followed by Example 8 |
| 153 | 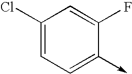 | 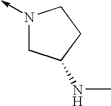 | LC-MS RT = 2.12 min m/z (APCI & ESI) 429/431 [MH⁺](6 min run) | RT followed by Example 8 |
| 154 | 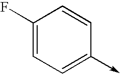 | 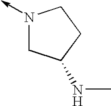 | LC-MS RT = 1.95 min m/z (APCI & ESI) 395 [MH⁺](6 min run) | RT followed by Example 8 |

Examples 155-157 were prepared according to the methods described above for example 16, starting from the appropriate pyridone¹ and the bromide from preparation 25.
1. The pyridones are either commercially available or known in the literature.

| | |
|---|---|
| | I |
| 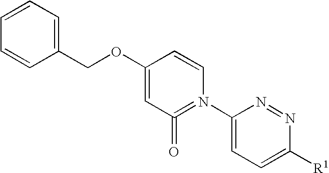 | |

| Exam- | | | |
|---|---|---|---|
| ple | R¹ | Analytical Data | Preparation |
| 155 | 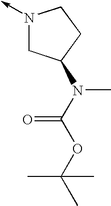 | LC-MS RT = 2.97 min m/z (APCI & ESI) 464 [MH⁺](6 min run) | Dioxane 110° C. + KI |
| 156 | 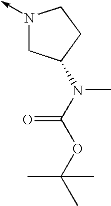 | LC-MS RT = 3.22 min m/z (APCI & ESI) 478 [MH⁺](6 min run) | Dioxane 110° C. + KI |
| 157 | 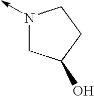 | LC-MS RT = 2.25 min m/z (APCI & ESI) 365 [MH⁺](6 min run) | Dioxane 110° C. + KI |

Examples 158-195 were prepared according to the methods described above for example 20 starting from the appropriate amine¹ and the appropriate acid chloride or chloroformate².
1. The starting amines are listed in the table below.

2. Acid chloride or chloroformate are commercially available.

| | |
|---|---|
| | I |
| 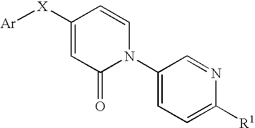 | |

| | | | | Preparation & |
|---|---|---|---|---|
| Example | Ar—X | R¹ | Analytical Data | Starting amine |
| 158 | 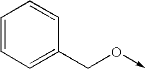 | 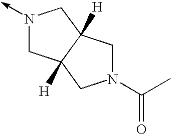 | ¹H NMR (400 MHz CDCl₃) δ ppm 2.13 (s, 3 H), 3.02-3.19 (m, 2 H), 3.37-3.41 (m, 2 H), 3.48-3.54 (m, 2 H), 3.74-3.82 (m, 4 H), 5.02 (s, 2 H), 6.01- 6.03 (m, 2 H), 6.41 (d, 1 H), 7.19 (dd, 1 H), 7.34-7.42 (m, | |
| # 5 H), 7.52 (dd, 1 H), 8.03 (s, 1 H). LRMS m/z (APCI & ESI) 431 [MH⁺] | As Example 20 from Example 8 | | | |
| 159 Racemic | 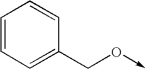 | 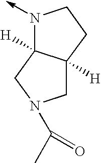 | LC-MS RT = 2.41 min m/z (APCI & ESI) 431 [MH⁺](6 min acidic run) | As Example 20 (Acetyl chloride) from Example 218 |
| 160 | 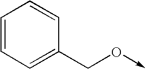 | 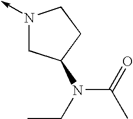 | ¹H NMR (400 MHz, CDCl₃) δ ppm 1.15-1.25 (m, 3 H), 2.07- 2.30 (br. m, 5 H). 3.23-3.40 (m, 2 H), 3.42 (q, 2 H), 3.66- 3.79 (m, 2 H), 4.42-5.10 (m, 3 H), 6.00-6.01 (m, 2 H), 6.40 (d, 1 H), 7.18 (dd, 1 H), 7.30- | |
| # 7.40 (m, 5 H), 7.49 (d, 1 H), 8.02 (s, 1 H). LRMS m/z (ESI) 433 [MH⁺] | As Example 20 (Acetyl Chloride) from Example 19 | | | |
| 161 | 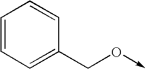 | 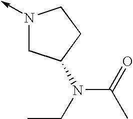 | ¹H NMR (400 MHz, CDCl₃) δ ppm 1.15-1.25 (m, 3 H), 2.07- 2.30 (br. m, 5 H). 3.23-3.40 (m, 2 H), 3.42 (q, 2 H), 3.66-3.79 (m, 2 H), 4.42-5.10 (m, 3 H), 6.00-6.01 (m, 2 H), 6.40 (d, 1 H), 7.18 (dd, 1 H), 7.30-7.40 | |
| # (m, 5 H), 7.49 (d, 1 H), 8.02 (s, 1 H). LRMS m/z (ESI) 433 [MH⁺] | As Example 20 (Acetyl Chloride) from Example 126 | | | |
| 162 | 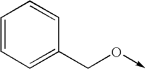 | 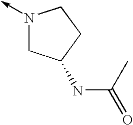 | LC-MS RT = 2.06 min m/z (APCI & ESI) 405 [MH⁺](6 min run) | As Example 20 (Acetyl Chloride) from Example 55 |
| 163 S, S | 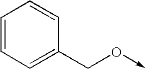 | 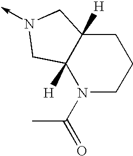 | LC-MS RT = 2.37 min m/z (ESI) 445 [MH⁺](6 min run) | As Example 20 (Acetyl Chloride) from Example 130 |
| 164 | 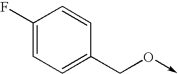 | 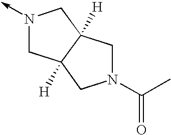 | LC-MS RT = 2.19 min m/z (APCI & ESI) 449 [MH⁺](6 min acidic run) | As Example 20 (Acetyl Chloride/DMF) from Example 125 |
| 165 | 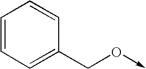 | 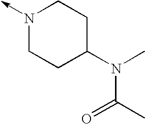 | LC-MS RT = 2.56 min m/z (APCI & ESI) 433 [MH⁺](6 min acidic run) | As Example 20 (Acetyl Chloride/DIPEA) from Example 230 |
| 166 | 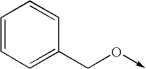 | 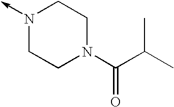 | LC-MS RT = 3.08 min m/z (ESI) 433 [MH⁺](6 min run) | As Example 20 (iPrCOCl) from Example 2 |
| 167 | 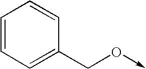 | 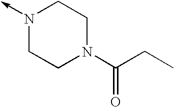 | LC-MS RT = 2.92 min m/z (ESI) 419 [MH⁺](6 min run) | As Example 20 (EtCOCl) from Example 2 |
| 168 | 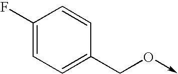 | 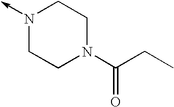 | LC-MS RT = 2.77 min m/z (ESI) 437 [MH⁺](6 min run) | As Example 20 (EtCOCl) from Example 124 |
| 169 | 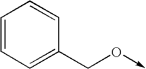 | 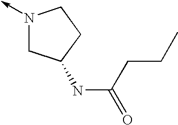 | LC-MS RT = 2.26 min m/z (APCI & ESI) 433 [MH⁺](6 min run) | As Example 20 (PrCOCl) from Example 55 |
| 170 | 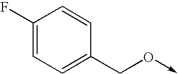 | 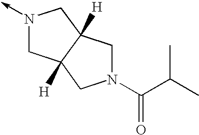 | LC-MS RT = 2.41 min m/z (APCI & ESI) 477 [MH⁺](6 min acidic run) | As Example 20 (iPrCOCl/DMF) from Example 125 |
| 171 | 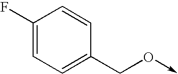 | 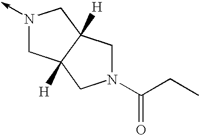 | LC-MS RT = 2.31 min m/z (APCI & ESI) 463 [MH⁺](6 min acidic run) | As Example 20 (EtCOCl/DMF) from Example 125 |
| 172 | 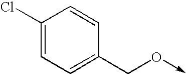 | 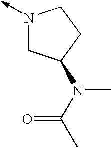 | LC-MS RT = 1.02 m/z (APCI) 453 [MH⁺](2 min run) | As Example 20 (Acetyl chloride) from Example 9 |
| 173 | 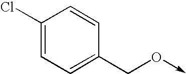 | 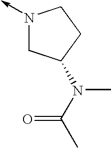 | ¹H NMR (400 MHz, CDCl₃) rotomers δ ppm 2.01-2.30 (m, 5 H), 2.93 (2 × s, 3 H), 3.38- 3.52 (m, 2 H), 3.64-3.79 (m, 2 H), 4.57-5.56 (m, 3 H), 6.01- 6.02 (m, 2 H), 6.41-6.43 (m, 1 H), 7.20 (d, 1 H), 7.39 (q, 4 H), 7.50-7.58 (m, 1 H), 8.06- | |
| # 8.10 (m, 1 H). LRMS m/z (ESI) 453 [MH⁺] | As Example 20 (Acetyl chloride) from Example 9 | | | |
| 174 | 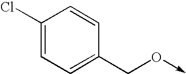 | 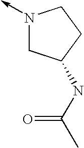 | ¹H NMR (400 MHz, CDCl₃) δ ppm 1.99 (s, 3 H), 2.00- 2.02 (m, 1 H), 2.29-2.37 (m, 1 H), 3.40 (dd, 1 H), 3.57-3.61 (m, 2 H), 3.75 (dd, 1 H), 4.59- 4.66 (m, 1 H), 5.00 (s, 2 H), 5.64 (d, 1 H), 6.00-6.02 (m, 2 H), 6.42 (d, 1 H), 7.20 (d, 1 H), 7.38 (q, 4 H), 7.50 (dd, | |
| # 1 H), 8.03 (d, 1 H). LRMS m/z (ESI) 439 [MH⁺] | As Example 20 (Acetyl chloride) from Example 203 | | | |
| 175 | 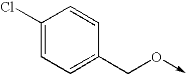 | 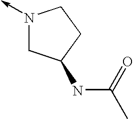 | ¹H NMR (400 MHz, CDCl₃) δ ppm 1.99-2.04 (m, 4 H), 2.29-2.37 (m, 1 H), 3.40 (dd, 1 H), 3.57-3.61 (m, 2 H), 3.75 (dd, 1 H), 4.59-4.66 (m, 1 H), 5.00 (s, 2 H), 5.64 (d, 1 H), 6.00-6.02 (m, 2 H), 6.42 (d, 1 H), 7.20 (d, 1 H), 7.38 (q, 4 H), 7.50 (dd, 1 H), 8.03 (d, 1 H). | |
| # LRMS m/z (ESI) 439 [MH⁺] | As Example 20 (Acetyl chloride) from Example 105 | | | |
| 176 | 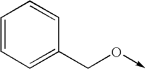 | 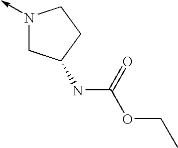 | ¹H NMR (400 MHz, CDCl₃) δ ppm 1.25 (t, 3 H), 1.97-2.05 (m, 1 H), 2.28-2.39 (m, 1 H), 3.41 (dd, 1 H), 3.52-3.62 (m, 2 H), 3.78 (dd, 1 H), 4.18 (q, 2 H), 4.40 (br. s, 1 H), 4.79 (br. s, 1 H), 5.02 (s, 2 H), 6.01- 6.02 (m, 2 H), 6.42 (d, 1 H), 7.20 (dd, 1 H), 7.33-7.43 (m, | |
| # 5 H), 7.50 (dd, 1 H), 8.06 (s, 1 H). LRMS m/z (ESI) 435 [MH⁺] | As Example 20 (EtOCOCl) from Example 55 | | | |
| 177 | 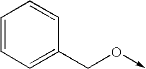 | 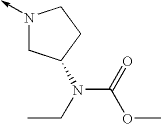 | LC-MS RT = 1.03 min m/z (ESI) 449 [MH⁺](2 min run) | As Example 20 (MeOCOCl) from Example 126 |
| 178 | 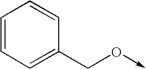 | 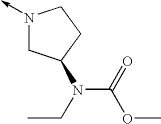 | LC-MS RT = 1.03 min m/z (ESI) 449 [MH⁺](2 min run) | As Example 20 (MeOCOCl) from Example 19 |
| 179 | 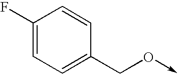 | 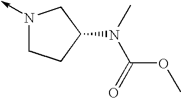 | LCMS RT = 2.58 min m/z (APCI) 453 [MH⁺](6 min run) | As Example 20 (MeOCOCl) from Example 123b |
| 180 | 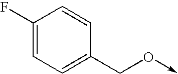 | 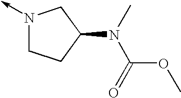 | LC-MS RT = 2.51 min m/z (APCI) 453 [MH⁺](6 min run) | As Example 20 (MeOCOCl) from Example 154 |
| 181 | 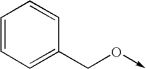 | 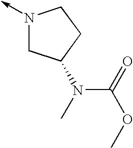 | LC-MS RT = 2.46 min m/z (APCI & ESI) 435 [MH⁺](6 min run) | As Example 20 (MeOCOCl) from Example 13 |
| 182 S, S | 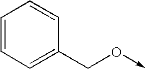 | 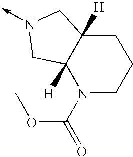 | LC-MS RT = 2.52 min m/z (ESI) 461 [MH⁺](6 min run) | As Example 20 (MeOCOCl) from Example 130 |
| 183 R, R | 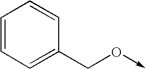 | 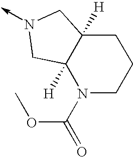 | LC-MS RT = 2.52 min m/z (ESI) 461 [MH⁺](6 min run) | As Example 20 (MeOCOCl) from Example 131 |
| 184 S, S | 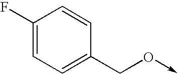 | 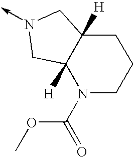 | LC-MS RT = 2.57 min m/z (ESI) 479 [MH⁺](6 min run) | As Example 20 (MeOCOCl) from Example 135 |
| 185 R, R | 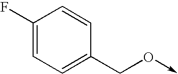 | 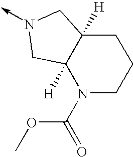 | LC-MS RT = 2.57 min m/z (ESI) 479 [MH⁺](6 min run) | As Example 20 (MeOCOCl) from Example 136 |
| 186 | 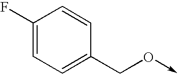 | 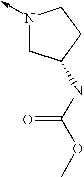 | LC-MS RT = 2.52 min m/z (ESI) 439 [MH⁺](6 min run) | As Example 20 (MeOCOCl) from Example 118 |
| 187 | 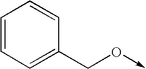 | 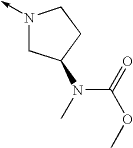 | LC-MS RT = 2.51 min m/z (APCI & ESI) 435 [MH⁺](6 min acidic run) | As Example 20 (MeOCOCl) from Example 15 |
| 188 | 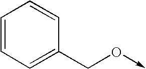 | 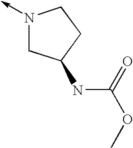 | LC-MS RT = 2.25 min m/z (APCI & ESI) 421 [MH⁺](6 min acidic run) | As Example 20 (MeOCOCl) from Example 54 |
| 189 Racemic | 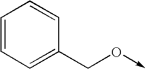 | 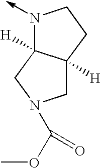 | LC-MS RT = 2.72 min m/z (APCI & ESI) 447 [MH⁺](6 min acidic run) | As Example 20 (MeOCOCl) from Example 218 |
| 190a Enantiomer a | 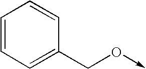 | 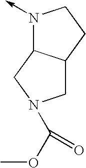 | LC-MS RT = 2.60 min m/z (APCI & ESI) 447 [MH⁺](6 min acidic run) | As Example 20 (MeOCOCl) from Example 218 |
| 190b Enantiomer b | 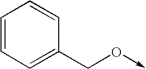 | 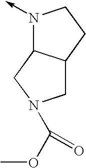 | LC-MS RT = 2.63 min m/z (APCI & ESI) 447 [MH⁺](6 min acidic run) | As Example 20 (MeOCOCl) from Example 218 |
| 191 | 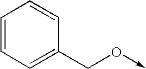 | 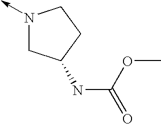 | LC-MS RT = 2.20 min m/z (APCI & ESI) 421 [MH⁺](6 min run) | As Example 20 (MeOCOCl) from Example 55 |
| 192 | 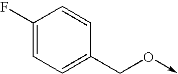 | 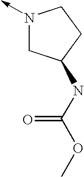 | ¹H NMR (400 MHz, CDCl₃) δ ppm 1.95-2.02 (m, 1 H), 2.22- 2.31 (m, 1 H), 3.35-3.61 (m, 7 H), 3.65-3.74 (m, 4 H), 4.32- 4.40 (m, 1 H), 4.98 (s, 2 H), 5.30-5.36 (m, 1 H), 6.01-6.05 (m, 2 H), 6.40 (d, 1 H), 7.07 | |
| # (t, 2 H), 7.17 (d, 1 H), 7.35- 7.39 (m, 2 H), 7.45-7.48 (m, 1 H), 8.02 (m, 1 H). LRMS m/z (APCI & ESI) 439 [MH⁺] | As Example 20 (MeOCOCl) from Example 143 | | | |
| 193 | 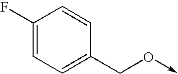 | 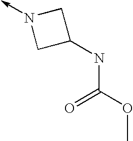 | ¹H NMR (400 MHz, CDCl₃) δ ppm 3.63 (s, 3 H) 3.78 (dd, 5.46 Hz, 2 H) 4.28 (q, 2 H) 4.58 (br. s., 1 H) 4.92 (s, 2 H) 5.38 (br. s., 1 H) 5.93-5.97 (m, 2 H) 6.27 (d, 1 H) 6.99-7.05 (m, 2 H) 7.10 (d, 1 H) 7.33 (m, 2 H) | |
| # 7.43 (dd, 1 H) 7.98 (d, 1 H) | As Example 20 (MeOCOCl) from Example 35 | | | |
| 194 | 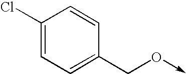 | 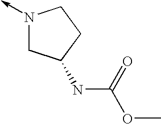 | ¹H NMR (400 MHz, CD₃OD) δ ppm 1.93-2.05 (m, 1 H), 2.21- 2.31 (m, 1 H), 3.28 (s, 3 H), 3.31-3.38 (m, 1 H), 3.44-3.65 (m, 2 H), 3.72 (dd, 1 H), 4.23- 4.31 (m, 1 H), 5.11 (s, 2 H), 6.04 (d, 1 H), 6.22 (dd, 1 H), | |
| # 6.54 (dd, 1 H), 7.36-7.50 (m, 6 H), 7.96 (m, 1 H) LRMS m/z (ESI) 455/457 [MH⁺] | As Example 20 (MeOCOCl) from Example 203 | | | |
| 195 | 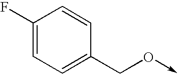 | 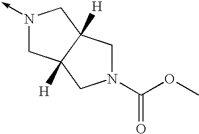 | LC-MS RT = 2.38 min m/z (APCI & ESI) 465 [MH⁺](6 min acidic run) | As Example 20 (MeOCOCl/DMF) from Example 125 |

### Examples 196-203

Examples 196-203 were prepared according to the methods described above for examples 21 & 23, starting from the appropriate Pyridone¹ and the appropriate iodide²
1. The pyridones are either commercially available or known in the literature.

2. The iodides are either known in the literature or described in Preparations 32, 33, 34, 35, 36, 39 or 42.

| | |
|---|---|
| | I |
| 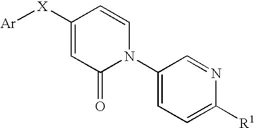 | |

| Example | Ar—X | R¹ | Analytical Data | Preparation |
|---|---|---|---|---|
| 196 | 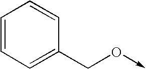 | 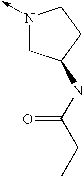 | LC-MS RT = 0.87 min m/z (APCI & ESI) 419 [MH⁺](2 min run) | As example 23 |
| 197 | 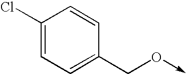 | 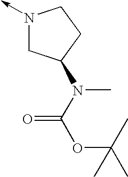 | ¹H NMR (400 MHz, CDCl₃) δ ppm 1.50 (s, 9 H), 2.06-2.26 (m, 2 H), 2.82 (s, 3 H), 3.40 (dd, 1 H), 3.45 (dd, 1 H), 3.66-3.71 (m, 2 H), 4.80-4.98 (br. s, 1 H), 5.00 (s, 2 H), 6.01-6.02 (m, 2 H), 6.42 (d, 1 H), 7.20 (d, 1 H), 7.38 | |
| # (q, 4 H), 7.51 (dd, 1 H), 8.03 (d, 1 H). LRMS m/z (ESI) 511 [MH⁺] | As example 23 | | | |
| 198 | 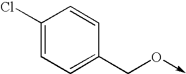 | 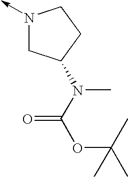 | ¹H NMR (400 MHz, CDCl₃) δ ppm 1.62 (s, 9 H), 2.06-2.24 (m, 2 H), 2.80 (s, 3 H), 3.37 (dd, 1 H), 3.45 (dd, 1 H), 3.64-3.71 (m, 2 H), 4.80-4.98 (br. s, 1 H), 5.00 (s, 2 H), 6.01-6.02 (m, 2 H), 6.41 (d, 1 H), 7.20 (d, 1 H), 7.38 | |
| # (q, 4 H), 7.50 (dd, 1 H), 8.03 (d, 1 H). LRMS m/z (ESI) 511 [MH⁺] | As example 23 | | | |
| 199 Racemic | 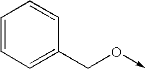 | 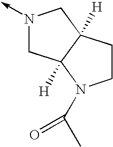 | LC-MS RT = 2.26 min m/z (APCI & ESI) 431 [MH⁺](6 min acidic run) | As example 21 |
| 200a Enantiomer a | 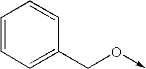 | 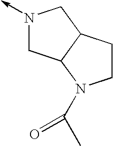 | LRMS m/z (APCI & ESI) 431 [MH⁺]Chiral prep HPLC (Chiralpak OJ-H, 250 × 21.2 mm id, 1:1 EtOH:MeOH, 15 ml/min r.t.) − RT = 9.18 min | As Example 21 |
| 200b Enantiomer b | 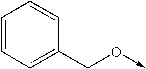 | 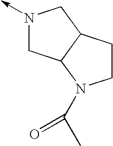 | LRMS m/z (APCI & ESI) 431 [MH⁺]Chiral prep HPLC (Chiralpak OJ-H, 250 × 21.2 mm id, 1:1 EtOH:MeOH, 15 ml/min r.t.) − RT = 11.30 min | As Example 21 |
| 201 Racemic | 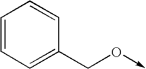 | 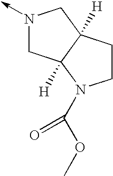 | LC-MS RT = 2.46 min m/z (APCI & ESI) 447 [MH⁺](6 min acidic run) | As example 21 but at 120° C. |
| 202a Enantiomer a | 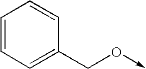 | 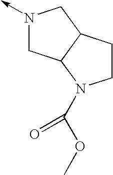 | LRMS m/z (APCI & ESI) 447 [MH⁺]Chiral prep HPLC (Chiralpak OJ-H, 250 × 21.2 mm id, 1:1 EtOH:MeOH, 15 ml/min r.t.) − RT = 13.09 min | As example 21 but at 120° C. |
| 202b Enantiomer b | 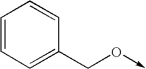 | 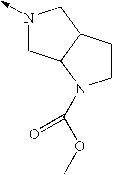 | LRMS m/z (APCI & ESI) 447 [MH⁺]Chiral prep HPLC (Chiralpak OJ-H, 250 × 21.2 mm id, 1:1 EtOH:MeOH, 15 ml/min r.t.) − RT = 16.51 min | As example 21 but at 120° C. |
| 203 | 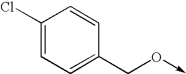 | 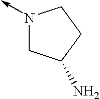 | ¹H NMR (400 MHz, CDCl₃) δ ppm 1.80-1.84 (m, 1 H), 2.20- 2.26 (m, 1 H), 3.22 (dd, 2 H), 3.48-3.53(m, 1 H), 3.61-3.78 (m, 2 H), 5.00 (s, 2 H), 6.00-6.02 (m, 2 H), 6.38-6.40 (m, 1 H), 7.20 (d, 1 H), 4.38 (q, 4 H), 7.46 (dd, 1 H), | |
| # 8.03 (d, 1 H). LRMS m/z (APCI) 397 [MH⁺] | As Example 23 followed by Example 8 | | | |

Examples 204-206 were prepared according to the method described below: 6′-[(3R)-3-{[tert-Butyl(dimethyl)silyl]oxy}pyrrolidin-1-yl]-2-oxo-2H-1,3′-bipyridin-4-yl 4-bromobenzenesulfonate from Preparation 47 (197 mg, 0.324 mmol), the appropriate benzyl alcohol (0.982 mmol) and potassium hydroxide (55 mg) in DMSO (3 ml) were heated at 130° C. under nitrogen for 1 hour then allowed to stand at r.t. overnight. The reactions were diluted with methanol (3 ml) and passed down a SCX column, washed with methanol and the product eluted with 2M NH₃ in methanol, evapourated to dryness. The residue was chromatographed on Biotage 12×150 mm silica column eluting with DCM/MeOH/NH₃ 98/2/0 to 93/7/1. Solvent removed in vacuo to give the title compound as solids.

| | |
|---|---|
| | I |
| 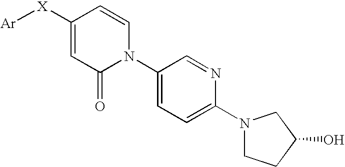 | |

| Ex- | | | |
|---|---|---|---|
| am- | | | Prepar- |
| ple | Ar—X | Analytical Data | ation |
| 204 | 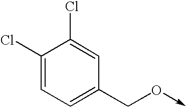 | LC-MS RT = 1.20 min m/z (APCI & ESI) 432 [MH⁺](2 min run) | As above |
| 205 | 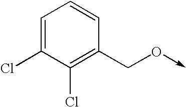 | LC-MS RT = 2.22 min m/z (APCI & ESI) 432/434 [MH⁺](6 min acidic run) | As above |
| 206 | 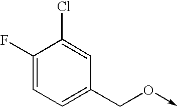 | ¹H NMR (400 MHz, CDCl₃) δ ppm 2.04- 2.20 (m, 2 H), 3.48- 3.65 (m, 5 H), 4.56- 4.63 (m, 1 H), 4.97 (s, 2 H), 5.99-6.05 (m, 2 H), 6.42 (d, 1 H), 7.15-7.21 (m, 2 H), 7.27-7.28 (m, 1 H), 7.46-7.50 (m, | |
| # 2 H), 8.05 (m, 1 H) | As above but using 60% NaH followed by HCl depro- tection of silyl group | | |

Examples 207-212 were prepared according to the methods described above for example 26 starting from the appropriate amine¹ and ketone or aldehyde².
1. The amines are described in Examples 8, 125, 130 and 131.

2. The ketones and aldehydes are commercial available

| | |
|---|---|
| | I |
| 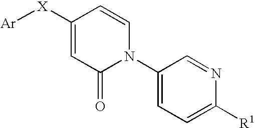 | |

| Example | Ar—X | R¹ | Analytical Data | Preparation |
|---|---|---|---|---|
| 207 | 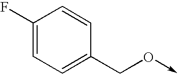 | 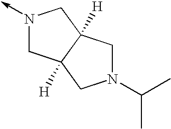 | LC-MS RT = 2.00 min m/z (APCI & ESI) 449 [MH⁺](6 min run) | As Example 26 |
| 208 | 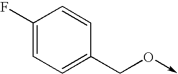 | 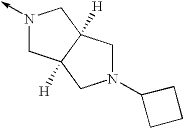 | LC-MS RT = 2.05 min m/z (APCI & ESI) 461 [MH⁺](6 min run) | As Example 26 |
| 209 | 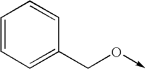 | 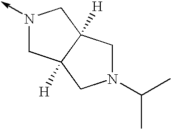 | LC-MS RT = 1.96 min m/z (APCI & ESI) 431 [MH⁺](6 min run) | As Example 26 |
| 210 | 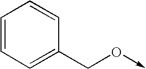 | 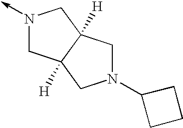 | LC-MS RT = 2.00 min m/z (APCI & ESI) 443 [MH⁺](6 min run) | As Example 26 |
| 211 S, S | 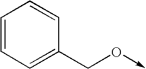 | 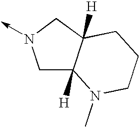 | ¹H NMR (400 MHz, CD₃OD) δ ppm 1.56- 1.64 (m, 1 H), 1.66- 1.85 (m, 3 H), 2.29 (td, 1 H), 2.33 (s, 3 H), 2.49-2.57 (m, 1 H), 2.72-2.79 (m, 1 H), 2.88-2.96 (m, 1 H), 3.47-3.53 (m, | |
| # 3 H), 3.80 (d, 1 H), 5.48 (s, 2 H), 6.07 (d, 1 H), 6.27 (dd, 1 H), 6.57 (d, 1 H), 7.30- 7.53 (m, 7 H), 7.97 (d, 1 H). | As Example 26 with AcOH instead of base | | | |
| 212 R, R | 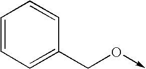 | 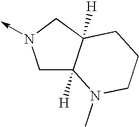 | ¹H NMR (400 MHz, CD₃OD) δ ppm 1.56- 1.64 (m, 1 H), 1.66- 1.85 (m, 3 H), 2.29 (td, 1 H), 2.33 (s, 3 H), 2.49-2.57 (m, 1 H), 2.72-2.79 (m, 1 H), 2.88-2.96 (m, 1 H), 3.47-3.53 (m, | |
| # 3 H), 3.80 (d, 1 H), 5.48 (s, 2 H), 6.07 (d, 1 H), 6.27 (dd, 1 H), 6.57 (d, 1 H), 7.30- 7.53 (m, 7 H), 7.97 (d, 1 H) LRMS m/z (APCI & ESI) 417 [MH⁺] | As Example 26 with AcOH instead of base | | | |

Examples 213-240 were prepared using the methods indicated in the table below starting from the fluoropyridine of preparation 2.

| | |
|---|---|
| | I |
| 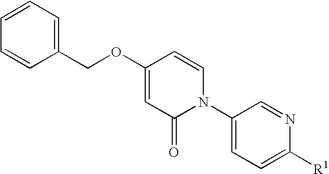 | |

| Ex- | | | |
|---|---|---|---|
| am- | | | |
| ple | R¹ | Analytical Data | Preparation |
| 213 | 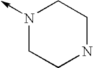 | LC-MS RT =2.402 min 363.1 [MH⁺]Method A | Example 3 at 140° C. followed by Example 17 |
| 214 | 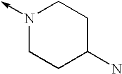 | LC-MS RT =2.228 min 377.2 [MH⁺]Method A | Example 3 at 140° C. followed by Example 17 |
| 215 S, S | 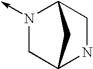 | LC-MS RT =2.356 min 375.2 [MH⁺]Method A | Example 3 at 140° C. followed by Example 17 |
| 216 | 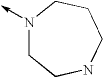 | LC-MS RT =2.405 min 377.2 [MH⁺]Method A | Example 3 at 140° C. followed by Example 17 |
| 217 | 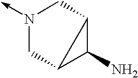 | LC-MS RT =2.104 min 375.2 [MH⁺]Method A | Example 3 at 140° C. followed by Example 17 |
| 218 R, R | 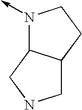 | LC-MS RT =2.397 min 389.2 [MH⁺]Method A | Example 3 at 140° C. followed by Example 17 |
| 219 | 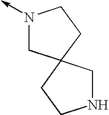 | LC-MS RT =1.884 min 403.3 [MH⁺]Method B | Example 3 at 140° C. followed by Example 17 |
| 220 | 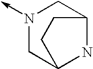 | LC-MS RT =2.223 min 389.1 [MH⁺]Method B | Example 3 at 140° C. followed by Example 17 |
| 221 | 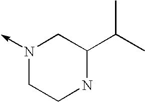 | LC-MS RT =2.333 min 405.3 [MH⁺]Method B | Example 3 at 140° C. followed by Example 17 |
| 222 | 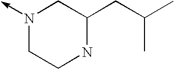 | LC-MS RT =2.455 min 419.3 [MH⁺]Method B | Example 3 at 140° C. followed by Example 17 |
| 223 | 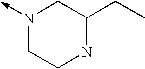 | LC-MS RT =2.302 min 391.3 [MH⁺]Method B | Example 3 at 140° C. followed by Example 17 |
| 224 Race- mic | 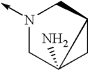 | LC-MS RT =2.203 min 375.2 [MH⁺]Method A | Example 3 at 140° C. followed by Example 17 |
| 225 Race- mic | 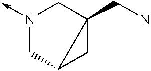 | LC-MS RT =2.159 min 389.2 [MH⁺]Method A | Example 3 at 140° C. followed by Example 17 |
| 226 Race- mic | 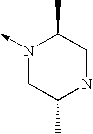 | LC-MS RT =2.254 min 391.1 [MH⁺]Method B | Example 3 at 140° C. followed by Example 17 |
| 227 Race- mic | 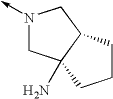 | LC-MS RT =2.113 min 403.3 [MH⁺]Method B | As Exam- ple 3 at 140° C. followed by Example 17 |
| 228 Race- mic | 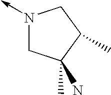 | LC-MS RT =2.07 min 391.3 [MH⁺]Method B | Example 3 at 140° C. followed by Example 17 |
| 229 Race- mic | 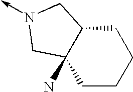 | LC-MS RT =2.174 min 417.1 [MH⁺]Method B | Example 3 at 140° C. followed by Example 17 |
| 230 | 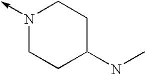 | LC-MS RT =2.37 min 391.3 [MH⁺]Method A | Example 3 at 140° C. followed by Example 17 |
| 231 | 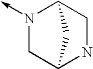 | LC-MS RT =2.267 min 375.2 [MH⁺]Method A | Example 3 at 140° C. followed by Example 17 |
| 232 | 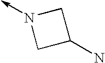 | LC-MS RT =2.148 min 349.2 [MH⁺]Method A | Example 3 at 140° C. followed by Example 17 |
| 233 | 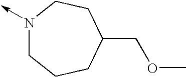 | LC-MS RT =2.304 min 420.2 [MH⁺]Method B | As Exam- ple 3 at 140° C. |
| 234 | 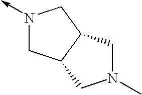 | LC-MS RT =2.259 min 403.2 [MH⁺]Method A | As Exam- ple 3 at 140° C. |
| 235 | 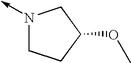 | LC-MS RT =2.078 min 378.2 [MH⁺]Method B | As Exam- ple 3 at 140° C. |
| 236 Race- mic | 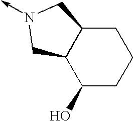 | LC-MS RT =2.058 min 418.2 [MH⁺]Method B | As Exam- ple 3 at 140° C. |
| 237 Race- mic | 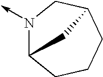 | LC-MS RT =2.226 min 388.2 [MH⁺]Method B | As Exam- ple 3 at 140° C. |
| 238 Race- mic | 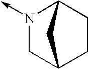 | LC-MS RT =2.094 min 374.2 [MH⁺]Method B | As Exam- ple 3 at 140° C. |
| 239 | 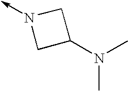 | LC-MS RT =2.077 min 377.3 [MH⁺]Method B | As Exam- ple 3 at 140° C. |
| 240 | 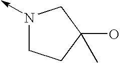 | LC-MS RT =1.926 min 378.2 [MH⁺]Method B | As Exam- ple 3 at 140° C. |

### Example 241

### =Preparation 23

### tert-Butyl (1-{4-[(4-fluorobenzyl)oxy]-2-oxo-2H-1,3′-bipyridin-6′-yl}-3-methylazetidin-3-yl) methylcarbamate

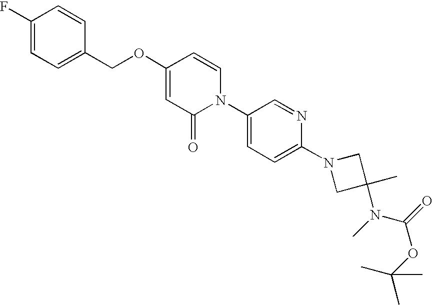

Prepared according to Preparation 22 from tert-butyl (1-{4-[(4-fluorobenzyl)oxy]-2-oxo-2H-1,3′-bipyridin-6′-yl}-3-methylazetidin-3-yl)carbamate from Example 120 to give the title compound as a solid. LC-MS RT=1.52 min m/z (APCI & ESI) 439 [MH⁺] (2 min run).

### Example 242

### =Preparation 22

### tert-Butyl {1-[4-(benzyloxy)-2-oxo-2H-1,3′-bipyridin-6′-yl]-3-methylazetidin-3-yl}methyl carbamate

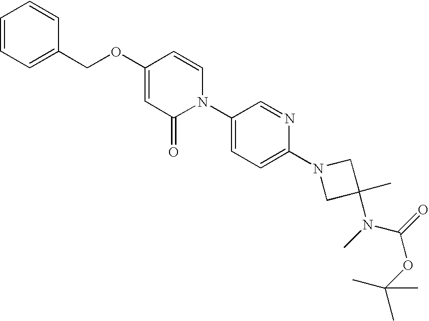

A solution of tert-butyl {1-[4-(benzyloxy)-2-oxo-2H-1,3′-bipyridin-6′-yl]-3-methylazetidin-3-ylcarbamate from Example 119 (100 mg, 2.16 mmol) in dry DMF (2 ml) was treated with sodium hydride (60% dispersion in oil, 17.3 mg, 0.432 mmol) at r.t. and stirred for 2 hours. Methyl iodide (26.9 ul, 0.432 mmol) was added and stirring continued for 2 hours. The reaction was carefully diluted with methanol (2 ml) and passed down a SCX column, washed with methanol and the product eluted with 2M NH3 in methanol. The solution was evaporated to dryness to give the title compound as a solid (91 mg, 88%) LC-MS RT=1.50 min m/z (APCI & ESI) 421 [MH⁺] (2 min run)

### Example 243

### =Preparation 55

### tert-Butyl {(3R)-1-[4-(benzyloxy)-2-oxo-2H-1,2′-bipyridin-5′-yl]pyrrolidin-3-yl}methylcarbamate

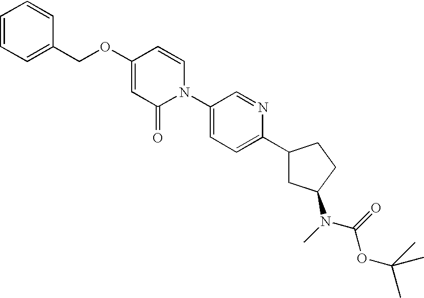

A suspension of 4-(Benzyloxy)-5′-iodo-2H-1,2′-pyridin-2-one from Preparation 2a (100 mg, 0.24 mmol), tert-butyl methyl[(3R)-pyrrolidin-3-yl]-carbamate (60 mg, 0.29 mmol; see Example 34b in WO2003/106462), Pd(OAc)₂ (2.5 mg, 0.009 mmol), BINAP (6.1 mg, 0.009 mmol) and NaOtBu (33 mg, 0.35 mmol) in toluene (5 ml) was purged with nitrogen for 30 mins. Reaction mixture was heated under reflux condition for 12 h. After cooling to room temperature, water was added and reaction mixture was extracted with ethyl acetate (3×20 ml). Organic layer was washed with water (30 ml), brine (30 ml) and dried (Na₂SO₄). Concentration and column purification (silica, 30% EtOAc/hexane) provided the title compound as a light yellow solid (100 mg, 84%) ¹H NMR (400 MHz, CDCl₃) δ ppm 1.47 (s, 9H), 2.13-2.22 (m, 2H), 2.80 (s, 3H), 3.23-3.33 (m, 2H), 3.46-3.52 (m, 2H), 5.02 (s, 2H), 6.02 (d, 1H), 6.06 (dd, 1H), 6.94 (dd, 1H), 7.34-7.40 (m, 5H), 7.59-7.65 (m, 2H), 7.80 (d, 1H). LRMS m/z (FIA) 477 [MH⁺]

### Preparation 1

### 6′-Amino-4-(benzyloxy)-2H-1,3′-bipyridin-2-one

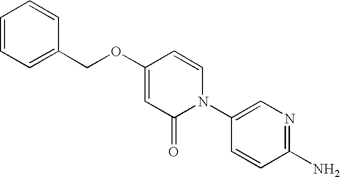

A stirred solution of 4-benzyloxy-1H-pyrid-2-one (5.0 g, 24.8 mmol; Aldrich), 5-iodo-pyridin-2-ylamine (5.7 g, 26.1 mmol; Aldrich), trans-N,N′-dimethylcyclohexane-1,2-diamine (0.71 g, 4.97 mmol), potassium carbonate (10.3 g, 74.5 mmol) and copper(I) iodide (0.95 g, 4.97 mmol) in toluene (100 ml) was purged with nitrogen for 30 minutes. The mixture was then heated at reflux for 3 hours. The toluene was then evaporated and the residue was stirred with water (500 ml) for 30 minutes. The solids were collected by filtration and washed two more times with water (2×500 ml), collecting the solids by filtration after each wash. The solids were slurried in toluene/methanol (300 ml of a 3:7 mixture) and then concentrated, which was then repeated a further two times to give the desired product as a dark grey powder (7.06 g, 97%) which was used without further purification. ¹H NMR (400 MHz, d₆-DMSO) δ ppm 5.13 (s, 2H), 6.00 (d, 1H), 6.14 (dd, 1H), 7.30-7.43 (m, 6H), 7.65 (d, 1H), 8.07 (m, 1H), 8.28 (d, 1H). LRMS m/z (APCI/ES) 294 [MH⁺].

### Preparation 2, 2a

### 4-(Benzyloxy)-6′-fluoro-2H-1,3′-bipyridin-2-one (Prep2)

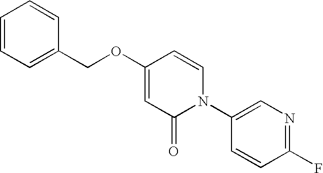 Method A

Pyridine (5 ml) was added dropwise to pyridine-HF solution (70% HF, 20 ml) CAUTION EXOTHERMIC. 6′-Amino-4-(benzyloxy)-2H-1,3′-bipyridin-2-on from Preparation 1 (3.5 g, 12.0 mmol) was added in one portion and the solution cooled to −78° C. under nitrogen. Sodium nitrite (1.0 g, 14.4 mmol) was added in one portion and the solution was allowed to warm to room temperature with stirring under nitrogen. Evolution of nitrogen began at −20° C. The mixture was poured into 10% aqueous potassium carbonate solution (200 ml) and the mixture was extracted with DCM (30×50 ml). The combined extracts were dried (MgSO₄), filtered and concentrated to give the desired product (Prep2) as a pink/brown amorphous powder (3.4 g, 96%). ¹H NMR (400 MHz, CD₃OD)₆ ppm 5.18 (s, 2H), 6.16 (s, 1H), 6.35 (d, 1H), 7.21 (d, 1H), 7.35-7.45 (m, 5H), 7.61 (d, 1H), 8.04 (m, 1H), 8.27 (s, 1H). LRMS m/z (APCI/ES) 297 [MH⁺].

Method B

A stirred solution of 4-benzyloxy-1H-pyrid-2-one (1.8 g, 9.0 mmol; Aldrich), 2-fluoro-5-iodo-pyridine (2.0 g, 9.0 mmol; Aldrich), trans-N,N′-dimethylcyclohexane-1,2-diamine (0.26 g, 1.79 mmol), potassium carbonate (3.70 g, 26.9 mmol) and copper(I) iodide (0.17 g, 0.90 mmol) in DMF (100 ml) was purged with nitrogen for 30 minutes then heated at 110° C. for 12 hours. The mixture was cooled to ambient temperature and filtered. The filtrate was evaporated and the residue was vigorously stirred with water (500 ml) for 3 hours. The solids were filtered off and dried by azeotroping with methanol/toluene (3:1, 3×400 ml) on a rotary evaporator. The crude residue was then purified by column chromatography eluting with a gradient from DCM to 9:1 DCM:MeOH to give the desired product (Prep2) as a white powder (1.18 g, 44%)

### 4-(Benzyloxy)-5′-iodo-2H-1,2′-pyridin-2-one (Prep2a)

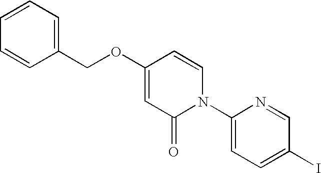

4-(Benzyloxy)-5′-iodo-2H-1,2′-bipyridin-2-one (Prep2a) was obtained as a by product from Method B as a colourless solid (3.6 g) ¹H NMR (400 MHz, d₆-DMSO) δ ppm 5.14 (s, 2H), 5.98 (d, 1H), 6.15 (dd, 1H), 7.35-7.46 (m, 5H), 7.61 (d, 1H), 7.82 (d, 1H), 8.29 (dd, 1H), 8.79 (d, 1H). LRMS m/z (APCI & ES) 405 [MH⁺]

### Preparation 3

### 4-(Benzyloxy)-6′-chloro-2H-1,3′-bipyridin-2-one

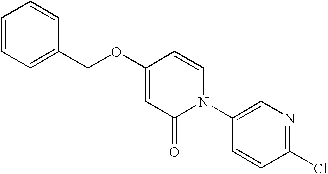

A stirred solution of 4-benzyloxy-1H-pyrid-2-one (200 mg, 1.0 mmol; Aldrich), 2-chloro-5-iodo-pyridine (239 mg, 1.0 mmol; Aldrich), trans-N,N′-dimethylcyclohexane-1,2-diamine (28 mg, 0.2 mmol), potassium carbonate (276 mg, 2.0 mmol) and copper(I) iodide (38 mg, 0.2 mmol) in toluene (1 ml) was heated at 110° C. for 18 hours in a reacti-vial. After cooling to room temperature, the mixture was stirred with DCM for 10 minutes. The mixture was filtered and the solids were washed with DCM. The combined organic components were concentrated and the residue was purified by column chromatography eluting with a gradient from 4:1 heptane:EtOAc to EtOAc, which gave the desired product as a white solid (125 mg, 40%). ¹H NMR (400 MHz, CDCl₃) [symbol] ppm 5.06 (s, 2H), 6.06 (d, 1H), 6.13 (dd, 1H), 7.20 (d, 1H), 7.35-7.47 (m, 6H), 7.80 (dd, 1H), 8.38 (dd, 1H). LRMS m/z (APCI & ES) 313 [M(³⁵Cl)H⁺].

### Preparation 4

### 4-Hydroxy-6′-[(3R)-3-hydroxypyrrolidin-1-yl]-2H-1,3′-bipyridin-2-one

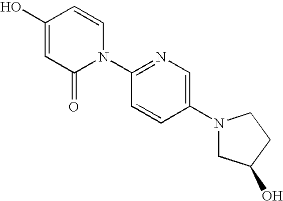

To a stirred solution of 4-(benzyloxy)-6′-[(3R)-3-hydroxypyrrolidin-1-yl]-2H-1,3′-bipyridin-2-one from Example 11 (1.65 g, 4.54 mmol) in ethanol (15 ml) was added 1-methyl-1,4-cyclohexadiene (2.20 g, 23.20 mmol) and 20% palladium hydroxide on carbon (190 mg). After heating at 70° C. for 3 hours the mixture was further diluted with ethanol (approximately 100 ml) until all of the reaction mixture other than the catalyst was in solution. The hot mixture was then filtered through arbocel under nitrogen, the catalyst was washed with ethanol (2×25 ml) and the filtrates were combined. The solution was concentrated and the residue was slurried with ethanol (10 ml). The solid was collected by filtration, washed with ethanol (10 ml) then diethyl ether (10 ml) and air-dried to give the desired product as an off-white solid (1.10 g, 89%). ¹H NMR (400 MHz, CD₃OD) δ ppm 2.00-2.09 (m, 1H), 2.10-2.21 (m, 1H), 3.43-3.49 (m, 1H), 3.55-3.65 (m, 3H), 4.51-4.55 (m, 1H), 5.85 (d, 1H), 6.12 (dd, 1H), 6.58 (d, 1H), 7.42 (d, 1H), 4.46 (dd, 1H), 7.98 (d, 1H). LRMS m/z (APCI) 275 [MH⁺]

### Preparation 5

### (3R)-1-{4-[(4-Chlorobenzyl)oxy]-2-oxo-2H-1,3′-bipyridin-6′-yl}pyrrolidin-3-yl methanesulfonate

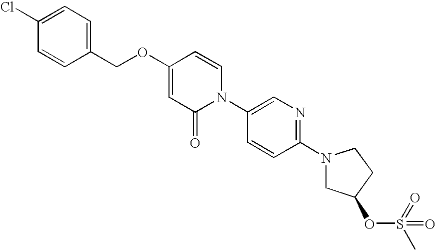

To a stirred solution of 4-[(4-chlorobenzyl)oxy]-6′-[(3R)-3-hydroxypyrrolidin-1-yl]-2H-1,3′-bipyridin-2-one from Example 10 (40 mg, 0.10 mmol) in dichloromethane (1 ml) was added triethylamine (28 μl, 0.20 mmol), followed by methanesulfonyl chloride (9 μl, 0.11 mmol). After stirring at room temperature for 15 hours, second portions of triethylamine (28 μl, 0.20 mmol) and methanesulfonyl chloride (9 μl, 0.11 mmol) were added. After 5 hours the reaction mixture was quenched by addition of water (2 ml) and extracted with dichloromethane (2 ml) using a phase separation cartridge. The organic component was concentrated to give a clear oil (50 mg). ¹H NMR analysis suggested the presence of some unreacted starting material. The crude product mixture was dissolved in dichloromethane (1 ml), then triethylamine (28 μl, 0.20 mmol) followed by methanesulfonyl chloride (9 μl, 0.11 mmol) were added. After 2 hours the mixture was diluted with water, the organic phase was separated and the aqueous layer was extracted with dichloromethane (2 ml). The organic components were combined, dried using a phase separation cartridge and concentrated to give a clear oil (48 mg, 100%) which solidified on standing. ¹H NMR (CDCl₃, 400 MHz) δ ppm 2.37-2.46 (m, 1H), 2.57-2.65 (m, 1H), 3.09 (s, 3H), 4.85-5.32 (m, 4H), 5.01 (s, 2H), 5.52 (br s, 1H), 6.01 (d, 1H), 6.12 (dd, 1H), 6.86 (d, 1H), 7.26-7.42 (m, 5H), 8.03 (dd, 1H), 8.25 (s, 1H). LRMS m/z (APCI) 476 [MH⁺]

### Preparation 6

### (3S)-1-(5-Bromopyridin-2-yl)pyrrolidin-3-ol

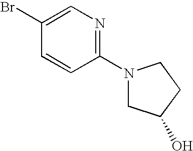

To a stirred solution of 2,5-dibromopyridine (1.0 g, 4.2 mmol) in tert-butanol (5 ml) was added (S)-3-hydroxypyrrolidine (0.74 g, 8.44 mmol; Aldrich) and sodium carbonate (1.34 g, 12.70 mmol). The mixture was heated at 140° C. for 3 hours in a reacti-vial. After cooling to room temperature the mixture was diluted with water (20 ml) and extracted with ethyl acetate (20 ml). The aqueous component was separated and extracted with ethyl acetate (20 ml). The combined organic components were dried (Na₂SO₄), filtered and concentrated to give a brown oil. The crude product mixture was purified by column chromatography (eluting with 100% DCM→90:10:1 DCM:MeOH:NH₃) to give the desired product as a white solid (1.0 g, 97%). ¹H NMR (400 MHz, CDCl₃) δ ppm 2.01-2.20 (m, 3H), 3.41-3.60 (m, 4H), 4.58-4.61 (m, 1H), 6.23 (d, 1H), 7.45 (dd, 1H), 8.11 (d, 1H). LRMS m/z (APCI) 377 [MH⁺].

### Preparation 7

### tert-Butyl[1-(5-iodopyridin-2-yl)-3-methylpyrrolidin-3-yl]carbamate

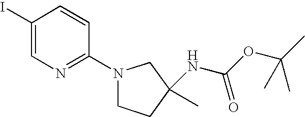

A solution of 2-fluoro-5-iodo pyridine (590 mg, 2.65 mmol) in DMSO (3 ml) was treated with potassium carbonate (731 mg, 5.3 mmol) and tert-butyl (3-methylpyrrolidin-3-yl)carbamate (Chem. Pharm. Bull 1996, 44(7) 1376, 583 mg, 2.91 mmol). The reaction was heated to 100° C. for 16 hours and then cooled to room temperature. Water (20 ml) was added and the product was extracted with ethyl acetate (2×20 ml). The combined organics were washed with brine (3×20 ml), dried (Na₂SO₄) and concentrated in vacuo to a brown oil. Purification on an SCX column (non-basic impurities with methanol, basic products eluted with 2M ammonia in methanol) gave the title compound as a pale brown oil (790 mg, 74%). ¹H NMR (400 MHz, CD₃OD) δ ppm 1.42 (s, 9H), 1.45 (s, 3H), 1.93-2.01 (m, 1H), 2.30-2.38 (m, 1H), 3.33-3.36 (m, 1H), 3.43-3.51 (m, 2H), 3.71-3.74 (d, 1H), 6.33-6.35 (d, 1H), 7.67-7.70 (dd, 1H), 8.14 (d, 1H) LRMS m/z (APCI) 404 [MH⁺].

### Preparation 10a; 10b

### tert-Butyl ethyl[(3R)-1-(5-iodopyridin-2-yl)pyrrolidin-3-yl]carbamate

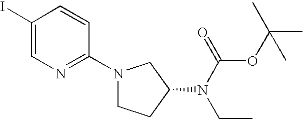

Potassium carbonate (1.45 g, 10.5 mmol) was added to a mixture of 2-fluoro-5-iodopyridine (780 mg, 3.50 mmol) and tert-butyl ethyl[(3R)-pyrrolidin-3-yl]carbamate (900 mg, 4.20 mmol) in DMSO (25 ml) at room temperature. The reagents were then heated at 100° C. for 5 hours and then cooled to room temperature. The reaction was diluted with water (20 ml) and extracted with EtOAc (50 ml). The organic phase was separated and the aqueous phase re-extracted with EtOAc (2×30 ml). All organic fractions were combined then dried (MgSO₄), filtered and evaporated to give a yellow oil Purification by chromatography (eluting with heptane→heptane:EtOAc (70:30)) gave the title compound (Prep. 10a) as a colourless solid 567 mg (39%). ¹H NMR (400 MHz, CDCl₃) δ ppm 1.11 (t, 3H), 1.42 (s, 9H), 2.07-2.21 (m, 2H), 3.10-3.38 (m, 4H), 3.59-3.66 (m, 2H), 4.60-4.71 (bm, 1H), 6.19 (d, 1H), 7.60 (dd, 1H), 8.25 (s, 1H). LRMS m/z (APCI) 418 [MH⁺]

The opposite enantiomer Prep. 10b was prepared via the same method with tert-butyl ethyl[(3S)-pyrrolidin-3-yl]carbamate to give the title compound as a colourless solid. ¹H NMR (400 MHz, CDCl3) δ ppm 1.11 (t, 3H), 1.42 (s, 9H), 2.07-2.21 (m, 2H), 3.10-3.38 (m, 4H), 3.59-3.66 (m, 2H), 4.60-4.71 (bm, 1H), 6.19 (d, 1H), 7.60 (dd, 1H), 8.25 (s, 1H). LRMS m/z (APCI) 418 [MH+]

### Preparation 12

### 6′-Amino-4-[(4-fluorobenzyl)oxy]-2H-1,3′-bipyridin-2-one

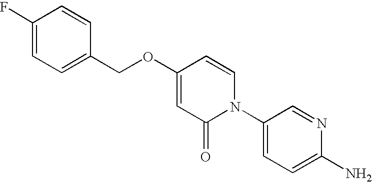

Prepared according to Preparation 1 from 4-[(4-fluorobenzyl)oxy]pyridin-2(1H)-one (WO2005018557) to give the title compound as a dark brown solid. ¹HNMR (400 MHz DMSO) [symbol] ppm 5.18 (s, 2H), 5.95 (s, 1H), 6.05 (d, 1H), 6.12-6.22 (br. m, 1H), 7.18-7.25 (m, 3H), 7.31-7.38 (m, 1H), 7.41-7.52 (m, 3H) LC-MS RT=1.89 m/z (APCI & ESI) 312 [MH⁺] (6 min acidic run)

### Preparation 13

### 6′-Fluoro-4-[(4-fluorobenzyl)oxy]-2H-1,3′-bipyridin-2-one

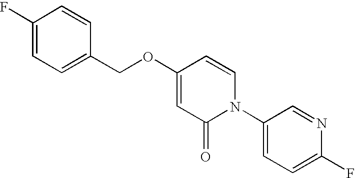

Prepared according to Preparation 2 Method A from 6′-Amino-4-[(4-fluorobenzyl)oxy]-2H-1,3′-bipyridin-2-one from Preparation 12 to give the title compound as a pale brown solid ¹HNMR (400 MHz d₆-DMSO) [symbol] ppm 5.12 (s, 2H), 6.01 (s, 1H), 6.14 (d, 1H), 7.23-7.35 (t, 2H), 7.31 (d, 1H), 7.49-7.52 (m, 2H), 7.64 (d, 1H) 8.04-8.08 (m, 1H), 8.28 (s, 1H) LC-MS RT=1.74 m/z (APCI & ESI) 315 [MH⁺] (6 min acidic run)

### Preparation 14

### 6′-Amino-4-[(4-chlorobenzyl)oxy]-2H-1,3′-bipyridin-2-one

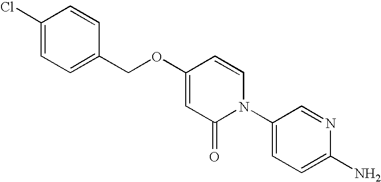

Prepared according to Preparation 1 from 4-[(4-chlorobenzyl)oxy]pyridin-2(1H)-one (WO2007018248) to give the title compound as a dark brown solid. ¹HNMR (400 MHz d₆-DMSO) [symbol] ppm 5.15 (s, 2H), 5.91 (s, 1H), 6.01 (d, 1H), 6.12-6.18 (br. m, 1H), 6.45-7.51 (br. m, 1H), 7.30-7.36 (d, 1H), 7.42-7.53 (m, 5H) LC-MS RT=1.99 m/z (APCI & ESI) 328 [MH⁺] (6 min acidic run)

### Preparation 15

### 4-[(4-chlorobenzyl)oxy]-6′-fluoro-2H-1,3′bipyridin-2-one

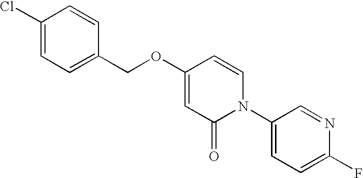

Prepared according to Preparation 2 Method A from 6′-Amino-4-[(4-chlorobenzyl)oxy]-2H-1,3′-bipyridin-2-one from Preparation 14 to give the title compound as a pale brown solid ¹HNMR (400 MHz d6-DMSO) [symbol] ppm 5.18 (s, 2H), 6.01 (s, 1H), 6.21 (d, 1H), 7.35 (d, 1H), 7.45-7.51 (m, 4H), 7.69 (d, 1H), 8.00-8.10 (m, 1H), 8.31 (s, 1H)

### Preparation 16

### 1-Benzyl-3-isopropylpyrrolidin-3-ol

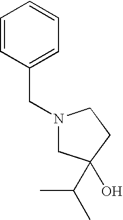

Cerium (III) chloride (4.4 g, 18.6 mmol) was suspended in anhydrous THF (62 ml) and stirred at r.t. overnight. The reaction was then cooled to 0° C.-5° C. and isopropylmagnesium chloride (2.0M in THF, 9.34 ml, 18.6 mmol) was added and the reaction was stirred at 0° C. for 1½ hours. 1-Benzyl-3-pyrrolidone (2.17 g/2 ml, 12.4 mmol; Aldrich) was added and the reaction was stirred over night allowing to warm to r.t. The reaction was poured into sat. NH₄Cl_{(aq)} (300 ml) and extracted with ethyl acetate (2×200 ml). The combined extracts were dried (MgSO₄) and concentrated to give the title compound as an oil (2.705 g, 99%). ¹H NMR (400 MHz, CDCl₃) δ ppm 0.91 (d, 3H), 0.95 (d, 3H), 1.65-1.72 (m, 1H), 1.74-1.81 (m, 1H), 1.87-1.94 (m, 1H), 2.28 (d, 1H), 2.30-2.34 (m, 1H), 2.70 (d, 1H), 2.96-3.05 (m, 1H), 3.65 (s, 2H), 7.21-7.34 (m, 5H). LRMS m/z (ESI) 220 [MH⁺]

### Preparation 17

### 3-Isopropylpyrrolidin-3-ol hydrochloride

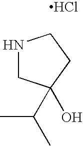

The hydrochloride salt of 1-Benzyl-3-isopropylpyrrolidin-3-ol from Preparation 16 (944 mg, 3.7 mmol) and 10% palladium on carbon (900 mg) were placed in a flask under N₂. Absolute ethanol (20 ml) was carefully added followed by acetic acid (0.63 ml, 11.1 mmol) and 1,4-cyclohexadiene (1.75 ml, 18.5 mmol). The mixture was heated to 50° C., once the reaction was complete by LCMS the reaction was cooled and filtered through celite, the filtrate was concentrated to give the title compound. LRMS m/z (ESI) 130 [MH⁺]

### Preparation 18

### (3S)—N-Isopropyl-N-methylpyrrolidin-3-amine hydrochloride

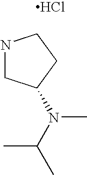

(S)-(−)-N-Boc-3-aminopyrrolidine (500 mg, 2.68 mmol; Lancaster) and acetone (10 ml) were added to 4:1 methanol:toluene (500 ml), the solution was then evaporated to dryness over a period of 5 hours at 35° C. The residue was taken up in DCM (100 ml), sodium triacetoxyborohydride (1.14 g) was added and the mixture stirred for 10 minutes. Acetic acid (20 ml) was added and the mixture stirred for 1 hour at r.t. MS gave [MH⁺] 228 as single component, corresponding to mono isopropyl secondary amine derivative.

Formaldehyde solution (4 ml) was added and the mixture stirred overnight. Aqueous potassium carbonate 10% (150 ml) was added and the mixture stirred for 1 hour. The DCM layer was separated, dried (MgSO4), then evaporated to give as a colourless viscous oil which partially solidified on standing to a waxy solid. The compound was then added to 4N HCl/dioxane (100 ml) and stirred overnight. The dioxane/HCl was evaporated and the residue dried overnight in vacuo at 50° C. to give the title compound as a solid (460 mg, 70%) ¹H NMR (400 MHz, CD₃OD) δ ppm 1.31 (s, 3H), 1.45 (s, 3H), 2.38-2.51 (m, 1H), 2.59-2.70 (m, 1H), 2.84 (s, 3H), 3.21-3.49 (m, 1H), 3.60-3.91 (m, 4H), 4.21-4.38 (m, 1H)

### Preparation 19

### tert-Butyl 3-azetidin-1-yl pyrrolidine-1-carboxylate

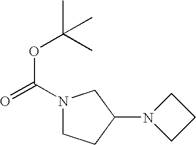

1-N-Boc-3-pyrrolidinone (400 mg, 2.16 mmol; Aldrich), azetidine hydrochloride (337 mg, 2.59 mmol; Aldrich) and sodium triacetoxyborohydride (915 mg, 4.32 mmol) were added to DCM (4 ml), acetic acid (40 ml) was then added dropwise over a period of 5 minutes with stirring. The mixture was then stirred overnight at r.t. 10% aqueous potassium carbonate solution (50 ml) was then added and the mixture stirred for 30 minutes. The DCM layer was separated, dried (MgSO4), then evaporated to give the title compound as an oil (451 mg, 92%). ¹H NMR (400 MHz, CD₃OD) δ ppm 1.42 (s, 9H), 1.62-1.75 (br. m, 1H), 1.80-1.93 (br. m, 1H), 2.09-2.19 (m, 2H), 2.58-2.62 (m, 1H), 3.25-3.40 (m, 8H) LRMS-m/z (APCI & ESI) 227 [MH⁺]

### Preparation 20

### 3-Azetidin-1-ylpyrrolidine hydrochloride

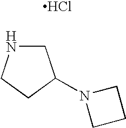

Prepared according to Example 8 from tert-Butyl 3-azetidin-1-ylpyrrolidine-1-carboxylate from Preparation 19 to give the title compound as colourless plates. ¹H NMR (400 MHz, CD₃OD) δ ppm ¹HNMR (400 MHz CD₃OD) [symbol] ppm 2.20-2.40 (m, 2H), 2.40-2.60 (m, 2H), 3.20-3.37 (m, 2H), 3.40-3.52 (m, 2H), 3.60-3.80 (m, 2H), 4.10-4.40 (m 3H)

### Preparation 21

### tert-Butyl (3-methylazetidin-3-yl)carbamate

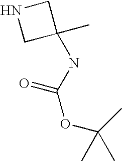

A solution of tert-butyl[1-(diphenylmethyl)-3-methylazetidin-3-yl]carbamate (1.397 g, 3.963 mmol) in a mixture of ethanol/ethyl acetate (25 ml/5 ml) was hydrogenated over pearlmans catalyst (300 mg) at 50 psi and 60° C. overnight. The catalyst removed by filtration and the filtrate reduced in vacuo to give a dark oil. The oil was taken up in methanol and passed down a SCX column, washed with methanol and the product washed off the column with 2M NH₃ in methanol. The solvent was evaporated to dryness to give a reddish oil which slowly solidified (730 mg, 99%) ¹H-NMR (400 MHz, CDCl₃) δ ppm 1.4 (s, 9H), 1.47 (s, 3H), 3.3 (d, 2H), 3.85 (br. s, 2H), 4.87

### Preparation 24

### tert-Butyl[(3S)-1-(4-hydroxy-2-oxo-2H-1,3′-bipyridin-6′-yl)pyrrolidin-3-yl]methylcarbamate

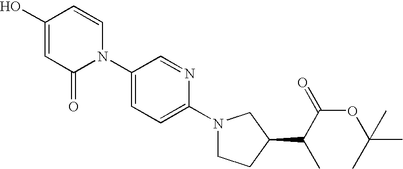

Prepared according to Preparation 4 from tert-Butyl {(3S)-1-[4-(benzyloxy)-2-oxo-2H-1,3′-bipyridin-6′-yl]pyrrolidin-3-yl}methyl carbamate from Example 12 to give the title compound as a white foam. ¹H NMR (400 MHz, CD₃OD) δ ppm 1.50 (s, 9H), 2.19-2.29 (m, 2H), 2.85 (s, 3H), 3.42-3.52 (m, 2H), 3.65-3.75 (m, 2H), 4.83-4.91 (m, 1H), 5.88 (d, 1H), 6.13 (dd, 1H), 6.62 (d, 1H), 7.46 (d, 1H), 7.53 (dd, 1H), 8.01 (d, 1H). LRMS m/z (APCI & ESI) 387 [MH⁺]

### Preparation 25

### tert-Butyl[(3R)-1-(6-bromopyridazin-3-yl)pyrrolidin-3-yl]carbamate

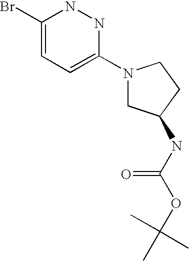

3,6-Dibromopyridazine (800 mg, 3.36 mmol; Maybridge), (3R)-(+)-3-(Boc-amino)pyrrolidine (689 mg, 3.70 mmol; Lancaster), cesium fluoride (102 mg, 0.673 mmol) and potassium carbonate (1.39 g, 10.1 mmol) were added to DMSO (10 ml) and the stirred mixture heated at 100° C. for 2 hours, effervescence was initially observed between 60-70° C. The mixture was poured into brine (50 ml) and the mixture stirred for 30 minutes. The solids were filtered off rinsed with water and sucked dry overnight on a sinter to give the title compound as a grey-brown solid (0.99 g, 86%) ¹H NMR (400 MHz, CD₃OD) δ ppm 1.41 (s, 9H), 1.93-2.06 (m, 1H), 2.21-2.30 (m, 1H), 3.31-3.38 (m, 1H), 3.42-3.61 (m, 2H), 3.69-3.75 (m, 1H), 4.20-4.29 (m, 1H), 6.85 (d, 1H), 7.48 (m, 1H)

### Preparation 26

### tert-Butyl[(3S)-1-(6-bromopyridazin-3-yl)pyrrolidin-3-yl]carbamate

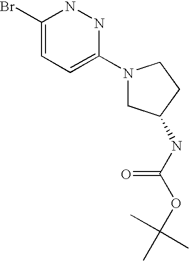

Prepared according to Preparation 25 from tert-butyl methyl[(3S)-pyrrolidin-3-yl]-carbamate (ECA International) to give the title compound as a buff solid. ¹H NMR (400 MHz, CDCl₃) δ ppm 1.44 (s, 9H), 2.10-2.29 (m, 2H), 2.82 (s, 3H), 3.41 (dd, 1H), 3.46 (dd, 1H), 3.67-3.78 (m, 2H), 4.84-4.95 (br. m, 1H), 6.55 (d, 1H), 7.29 (d, 1H). LRMS m/z (APCI/ESI) 359 [MH⁺]

### Preparation 27

### (3R)-1-(6-Bromopyridazin-3-yl)pyrrolidin-3-ol

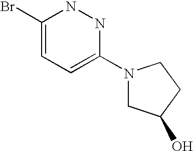

Prepared according to Preparation 25 from (R)-(+)-3-hydroxypyrrolidine (Aldrich) to give the title compound as a pale yellow solid. ¹H NMR (400 MHz, CDCl₃) δ ppm 1.60-1.80 (br. s, water+OH), 2.10-2.22 (m, 2H), 3.58-3.70 (m, 4H), 4.63-4.70 (m, 1H), 6.58 (d, 1H), 7.29 (d, 1H). LRMS m/z (APCI) 246 [MH⁺]

### Preparation 29

### Methyl 1-(5-iodopyridin-2-yl)pyrrolidine-3-carboxylate

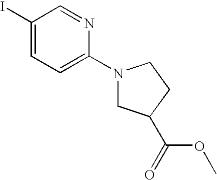

Prepared according to Preparation 7 from methylpyrrolidine-3-carboxylate (Bioorganic Med. Chem. Letts 14(19), 4861-4866, 2004) to give the title compound as an off-white solid. ¹H NMR (400 MHz, CDCl₃) δ ppm 2.25 (q, 2H), 3.17-3.22 (m, 1H), 3.39-3.43 (m, 1H), 3.51-3.59 (m, 1H), 3.60-3.66 (m 2H), 3.68 (s, 3H), 6.20 (d, 1H), 7.60 (dd, 1H), 8.22 (s, 1H). LRMS m/z (APCI & ESI) 333 [MH⁺]

### Preparation 30

### Methyl 1-[4-(benzyloxy)-2-oxo-2H-1,3′-bipyridin-6′-yl]pyrrolidine-3-carboxylate

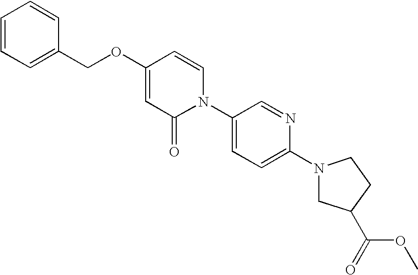

Prepared according to Preparation 2 Method B from methyl 1-(5-iodopyridin-2-yl)pyrrolidine-3-carboxylate from Preparation 29 to give the title compound as an off white solid. ¹H NMR (400 MHz, CDCl₃) δ ppm 2.24-2.32 (m, 2H), 3.18-3.23 (m, 1H), 3.44-3.51 (m, 1H), 3.59-3.64 (m, 1H), 3.65-3.79 (m, 5H), 5.01 (s, 2H), 6.00-6.02 (m, 2H), 6.40 (d, 1H), 7.18 (d, 1H), 7.31-7.41 (m, 5H), 7.46 (dd, 1H), 8.02 (s, 1H). LRMS m/z (APCI & ESI) 406 [MH⁺]

### Preparation 31

### 1-[4-(Benzyloxy)-2-oxo-2H-1,3′-bipyridin-6′-yl]pyrrolidine-3-carboxylic acid

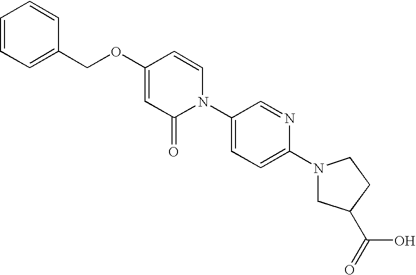

Sodium hydroxide (1M aq soln) (5.92 ml, 5.92 mmol) was added to a solution of methyl 1-[4-(benzyloxy)-2-oxo-2H-1,3′-bipyridin-6′-yl]pyrrolidine-3-carboxylate from Preparation 30 (1.20 g, 2.96 mmol) in MeOH (6.00 ml) and THF (4.00 ml) at r.t. under N₂. Stirring was continued for 3 hours and then the solvents were evaporated in vacuo, and the residue was partitioned between CH₂Cl₂ (10 ml) and water (10 mL). The aqueous layer was then acidified to pH2 by addition of 2M HCl (aq) (2 mL). Solvents were again removed in vacuo and the residue was azeotroped with toluene (2×10 mL) to give the title compound as an off white solid (1.37 g, 108%) ¹H NMR (400 MHz, CD₃OD) δ ppm 2.37-2.50 (m, 2H), 3.39-3.45 (m, 1H), 3.67-3.76 (m, 2H), 3.82-3.91 (m, 2H), 5.13 (s, 2H), 6.03 (d, 1H), 6.25 (dd, 1H), 7.18 (d, 1H), 7.30-7.43 (d, 5H), 7.52 (d, 1H), 7.99 (dd, 1H), 8.06 (d, 1H). LRMS m/z (APCI) 390 [MH⁺]

### Preparation 32

### tert-Butyl[(3S)-1-(5-iodopyridin-2-yl)pyrrolidin-3-yl]carbamate

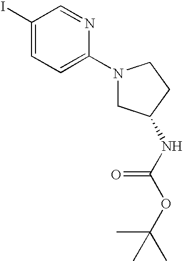

Prepared according to Preparation 7 from (S)-(−)-3-(Boc-amino)pyrrolidine (Lancaster) to give the title compound as a tan solid. ¹H NMR (300 MHz, CDCl₃) δ ppm 1.44 (s, 9H), 1.8-2.0 (m, 1H), 2.2-2.29 (m, 1H), 3.3 (d, 1H), 3.49-3.55 (m, 2H), 3.65-3.70 (m, 1H), 4.33 (br. s, 1H), 4.68 (br. s, 1H), 6.20 (d, 1H), 7.62 (d, 1H), 8.27 (s, 1H). LRMS m/z (ESI) 390 [MH⁺]

### Preparation 33

### (3S)-1-(5-Iodopyridin-2-yl)pyrrolidin-3-amine hydrochloride

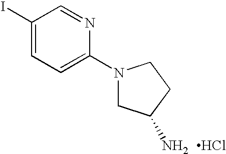

Prepared according to Example 13 from tert-Butyl[(3S)-1-(5-iodopyridin-2-yl) pyrrolidin-3-yl]carbamate from Preparation 32 to give the title compound as a white solid. ¹H NMR (300 MHz, CD₃OD) δ ppm 2.25-2.4 (m, 1H), 2.5-2.63 (m, 1H), 3.69-3.85 (m, 3H), 3.91-4.02 (m, 1H), 4.16 (br. s, 1H), 6.98 (d, 1H), 8.18 (s, 1H), 8.20 (s, 1H). LRMS m/z (ESI) 290 [MH⁺]

### Preparation 34

### Methyl[(3S)-1-(5-iodopyridin-2-yl)pyrrolidin-3-yl]carbamate

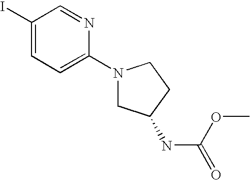

(3S)-1-(5-Iodopyridin-2-yl)pyrrolidin-3-amine hydrochloride from Preparation 33 (1.00 g, 2.76 mmol) was dissolved in DCM (15 ml) and treated with triethylamine (1.35 ml, 9.67 mmol). The resulting solution was stirred under nitrogen at r.t. for 30 min at 0° C. Methylchloroformate (362 uL, 2.9 mmol) was then added and the ice bath was removed. The resulting solution was stirred at r.t. over night. LCMS shows the desired product as the major peak. The mixture was washed with a saturated NaHCO₃ solution (2×10 ml), dried over (Na₂SO₄) filtered and evaporated to afford an orange oil. The oil was preabsorbed on silica and purified by chromatography eluting with EtOAc/heptane from 25/75 to 50/50 to give the title compound as a white solid (after trituration with ether), (620 mg 64%) ¹H-NMR (400 MHz, CD₃OD) δ ppm 1.90-2.05 (m, 1H), 2.16-2.30 (m, 1H), 3.23-3.33 (m, 1H), 3.39-3.53 (m, 2H), 3.61-3.69 (m, 4H), 4.21-4.32 (m, 1H), 6.32 (d, 1H), 7.67 (d, 1H), 8.13 (s, 1H). LC-MS RT=2.05 min m/z (APCI & ESI) 347.8 [MH⁺] (6 min acidic run)

### Preparation 35

### N-[(3R)-1-(5-Iodopyridin-2-yl)pyrrolidin-3-yl]propanamide

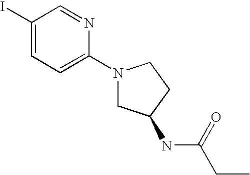

Prepared according to Preparation 7 from N-[(3R)-pyrrolidin-3-yl]propanamide (EP391169 A2) to give the title compound as a cream solid. ¹H NMR (400 MHz, CDCl₃) δ ppm 1.14 (t, 3H), 1.91-1.99 (m, 1H), 2.16 (q, 2H), 2.22-2.31 (m, 1H), 3.25 (dd, 1H), 3.43-3.54 (m, 2H), 3.65 (dd, 1H), 4.57-4.62 (m, 1H), 5.55 (br. s, 1H), 6.20 (d, 1H), 7.61 (dd, 1H), 8.25 (s, 1H). LRMS m/z (APCI & ESI) 346 [MH⁺]

### Preparation 36

### tert-Butyl[(3R)-1-(5-iodopyridin-2-yl)pyrrolidin-3-yl]methylcarbamate

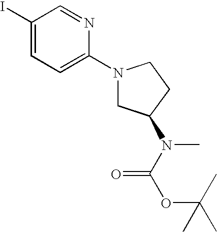

Prepared according to Preparation 7 from (R)-(+)-3-(Boc-amino)pyrrolidine (Lancaster) to give the title compound as an off white solid. ¹H NMR (300 MHz, CDCl₃) δ ppm 1.44 (s, 9H), 2.01-2.21 (m, 2H), 2.80 (s, 3H), 3.25-3.4 (m, 2H), 3.56-3.6 (m, 2H), 4.86 (br. s, 1H), 6.19 (d, 1H), 7.62 (d, 1H), 8.27 (s, 1H). LRMS m/z (ESI) 404 [MH⁺]

### Preparation 37

### tert-Butyl (3aRS,6aRS)-1-acetylhexahydropyrrolo[3,4-b]pyrrole-5(1H)-carboxylate

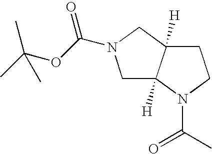

A solution of tert-butyl (3aRS,6aRS)-hexahydropyrrolo[3,4-b]pyrrole-5(1H)-carboxylate (WO2001081347, 2.00 g, 9.42 mmol) in dichloromethane (20 ml) was cooled to 0° C. and treated with triethylamine (1.58 ml, 11.3 mmol), followed by acetyl chloride (0.703 ml, 9.89 mmol). The cold bath was removed and the solution stirred at r.t. under nitrogen overnight. The reaction mixture was washed with sat. NaHCO₃ solution (2×20 ml) then brine (20 ml). The organics were dried (Na₂SO₄) and concentrated in vacuo to give the title compound as a pale amber oil (2.17 g, 90%) ¹H-NMR (400 MHz, CD₃OD) δ ppm 1.44 (s, 9H), 1.80-1.89 (m, 1H), 2.06 (s, 3H), 2.07-2.14 (m, 1H), 2.91-2.98 (m, 1H), 3.22-3.27 (m, 1H), 3.43-3.48 (m, 1H), 3.49-3.66 (br. m, 4H), 3.32-3.39 (m, 1H). LC-MS RT=2.45 min m/z (APCI & ESI) 155 [MH⁺-Boc] (6 min acidic run)

### Preparation 38

### (3aRS,6aRS)-1-Acetyloctahydropyrrolo[3,4-b]pyrrole

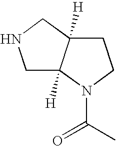

Prepared according to Example 17a from tert-butyl (3aRS,6aRS)-1-acetylhexahydropyrrolo[3,4-b]pyrrole-5(1H)-carboxylate from Preparation 37 to give the title compound as an amber oil which was used without further purification. ¹H-NMR (400 MHz, CD₃OD) δ ppm 1.79-1.86 (m, 1H), 2.05 (s, 3H), 2.09-2.15 (m, 1H), 2.70-2.76 (m, 1H), 2.83-2.88 (m, 1H), 2.99-3.06 (m, 3H), 3.57-3.64 (m, 2H), 4.30-4.36 (m, 1H). LRMS m/z (APCI & ESI) 155 [MH⁺]

### Preparation 39

### (3aRS,6aRS)-1-Acetyl-5-(5-iodopyridin-2-yl)-octahydropyrrolo[3,4-b]pyrrole

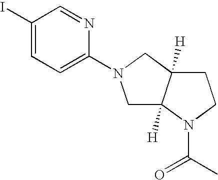

Prepared according to Preparation 7 from (3aRS,6aRS)-1-acetyloctahydropyrrolo[3,4-b]pyrrole from Preparation 38 to give the title compound as a golden oil which was used without further purification. ¹H-NMR (400 MHz, CD₃OD) δ ppm 1.89-1.97 (m, 1H), 2.07 (s, 3H), 2.13-2.23 (m, 1H), 3.05-3.14 (m, 1H), 3.34-3.39 (m, 1H), 5.54-3.69 (m, 5H) 4.47-4.55 (m, 1H), 6.37 (d, 1H), 7.70 (dd, 1H), 8.17 (d, 1H). LC-MS RT=2.15 min m/z (APCI & ESI) 358 [MH⁺] (6 min acidic run)

### Preparation 40

### 5-tert-Butyl 1-methyl (3aRS,6aRS)-hexahydropyrrolo[3,4-b]pyrrole-1,5-dicarboxylate

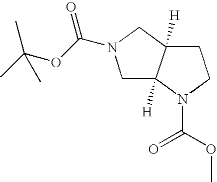

Prepared according to Preparation 37 using methyl chloroformate to give the title compound as a pale golden oil. ¹H-NMR (400 MHz, CD₃OD) 1.44 (s, 9H), 1.73-1.82 (m, 1H), 2.00-2.07 (m, 1H), 2.92-3.01 (m, 1H), 3.18-3.26 (m, 1H), 3.41-3.59 (br. m, 5H), 3.69 (s, 3H), 4.21-4.28 (m, 1H). LC-MS RT=2.82 min m/z (APCI) 171[MH⁺-Boc] (6 min acidic run)

### Preparation 41

### Methyl (3aRS,6aRS)-hexahydropyrrolo[3,4-b]pyrrole-1(2H)-carboxylate

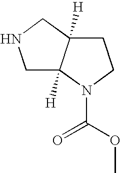

Prepared according to Example 17a from 5-tert-butyl 1-methyl (3aRS,6aRS)-hexahydropyrrolo[3,4-b]pyrrole-1,5-dicarboxylate from Preparation 40 to give the title compound as a pale yellow oil. ¹H-NMR (400 MHz, CD₃OD) δ ppm 1.69-1.77 (m, 1H), 1.98-2.07 (m, 1H), 2.65-2.71 (dd, 1H), 2.81-2.89 (br. m, 1H), 2.91-2.99 (m, 3H), 3.38-3.46 (m, 1H), 3.49-3.55 (m, 1H), 3.70 (br. s, 3H), 4.17-4.22 (m, 1H). LRMS m/z (APCI & ESI) 171 [MH⁺]

### Preparation 42

### Methyl (3aRS,6aRS)-5-(5-iodopyridin-2-yl)hexahydropyrrolo[3,4-b]pyrrole-1(2H)-carboxylate

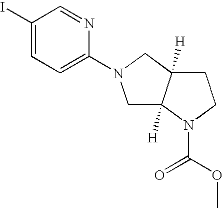

Prepared according to Preparation 7 from methyl (3aRS,6aRS)-hexahydropyrrolo[3,4-b]pyrrole-1(2H)-carboxylate from Preparation 41 to give the title compound as a colourless oil. ¹H-NMR (400 MHz, CD₃OD) δ ppm 1.82-1.91 (m, 1H), 2.05-2.15 (m, 1H), 3.09-3.17 (m, 1H), 3.30-3.35 (m, 1H), 3.44-3.54 (m, 1H), 3.56-3.73 (m, 8H), 4.37-4.44 (m, 1H), 6.39 (t, 1H), 7.71 (d, 1H), 8.17 (d, 1H). LRMS m/z (APCI & ESI) 374 [MH⁺]

### Preparation 43

### 4-Hydroxy-6′-pyrrolidin-1-yl-2H-1,3′-bipyridin-2-one

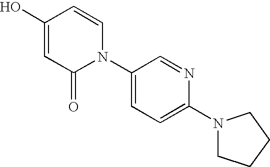

Prepared according to Preparation 4 from 4-(benzyloxy)-6′-pyrrolidin-1-yl-2H-1,3′-bipyridin-2-one example 34 to give the title compound as a solid which was used without further purification. LC-MS RT=0.59 min m/z (APCI & ESI) 258 [MH⁺]

### Preparation 44

### 2-Oxo-6′-pyrrolidin-1-yl-2H-1,3′-bipyridin-4-yl 4-bromobenzenesulfonate

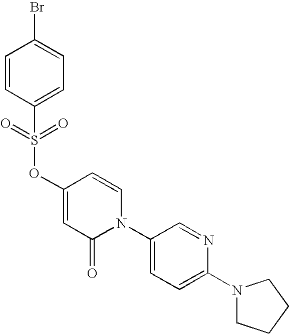

4-Hydroxy-6′-pyrrolidin-1-yl-2H-1,3′-bipyridin-2-one from Preparation 43 (1.0 g, 3.887 mmol), 4-bromobenzenesulfonyl chloride (1.15 g, 4.50 mmol) and potassium carbonate (1.07 g, 7.77 mmol) in dimethylacetamide (10 ml) under N₂ was stirred together overnight at ambient temperature (very thick reaction mixture observed in the morning). EtOAc (30 ml) and water (20 ml) was added and the mixture was stirred—incomplete solution (other than catalyst). Addition of DCM (50 ml) gave solution. The mixture was filtered through Arbocel, washing with DCM (50 ml), then water (50 ml). The layers were separated and the aqueous extracted with EtOAC (50 ml), bulked organic solution was washed water (3×50 ml), brine (50 ml), dried (MgSO₄) and evaporated. The residue recrystallized from MeCN (needed ˜100 ml) to give the title compound as a cream solid. (1.39 g, 75%) ¹H-NMR (400 MHz, CDCl₃) δ ppm 2.02 (m, 4H), 3.47 (m, 4H), 6.18-6.21 (m, 2H), 6.40 (d, 1H), 7.32 (d, 1H), 7.44 (dd, 1H), 7.75 (m, 2H), 7.82 (m, 2H), 8.03 (d, 1H). LRMS m/z (ESI) 476/478 [MH⁺]

### Preparation 45

### 4-(Benzyloxy)-6′-[(3R)-3-{[tert-butyl(dimethyl)silyl]oxy}pyrrolidin-1-yl]-2H-1,3′-bipyridin-2-one

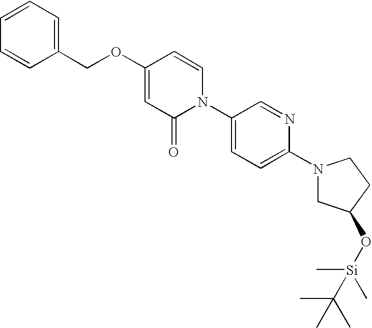

4-(Benzyloxy)-6′-[(3R)-3hydroxypyrrolidin-1-yl]-2H-1,3′-bipyridin-2-one from Example 11 (4.00 g, 11.01 mmol), imidazole (1.5 g, 22 mmol) and DMAP (53.8 mg, 0.44 mmol) were added to acetonitrile (100 ml). TBDMS-triflate (3.2 g, 12.1 mmol) was then added dropwise and the mixture was then heated at 50° C. for 1 hour. Mixture cooled back to r.t. and acetonitrile evaporated, residue stirred with water (200 ml) for 1 hour, mixture then extracted with EtOAc (3×200 ml). EtOAc solution then washed successively with 10% K2CO3 (aq) (3×200 ml), water (3×200 ml) then brine (200 ml). The EtOAc solution was then dried (MgSO4) and evaporated to give the title compound as a white amorphous powder (4.15 g, 78%) ¹H-NMR (400 MHz, d₆-DMSO) δ ppm 0.05 (d, 6H), 0.78 (s, 9H), 1.78-1.84 (m, 1H), 1.97-2.06 (m, 1H), 3.18-3.21 (m, 1H), 3.34-3.44 (m, 2H), 3.49-3.53 (m, 1H), 4.54-4.59 (m, 1H), 5.04 (s, 2H), 5.86 (s, 1H), 6.18 (d, 1H), 6.49 (d, 1H), 7.35-7.58 (m, 7H), 7.99 (s, 1H) LRMS m/z (ESI) 478 [MH⁺]

### Preparation 46

### 6′-[(3R)-3-{[tert-Butyl(dimethyl)silyl]oxy}pyrrolidin-1-yl]-4-hydroxy-2H-1,3′-bipyridin-2-one

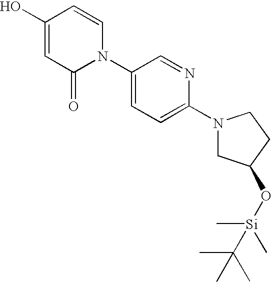

4-(Benzyloxy)-6′-[(3R)-3-{[tert-butyl(dimethyl)silyl]oxy}pyrrolidin-1-yl]-2H-1,3′-bipyridin-2-one from Preparation 45 (4.0 g, 8.376 mmol) in ethanol (100 ml) was hydrogenation over 10% palladium on carbon (1.0 g) at 50 psi and r.t. for 12 hours. The mixture filtered through celite, and the ethanol evaporated to give the title compound as a light brown amorphous solid (3.06 g, 94%) ¹H-NMR (400 MHz, d₆-DMSO) δ ppm 0.05 (s, 6H), 0.82 (s, 9H), 1.80-1.94 (m, 1H), 2.05-2.18 (m, 1H), 3.25-3.32 (m, 1H), 3.40-3.51 (m, 2H), 3.56-3.62 (m, 1H), 4.52-4.59 (m, 1H), 5.60 (s, 1H), 5.91 (d, 1H), 6.43 (d, 1H), 7.43 (d, 2H), 7.98 (s, 1H), 10.69 (br. s, 1H) LRMS m/z (ESI) 388 [MH⁺]

### Preparation 47

### 6′-[(3R)-3-{[tert-Butyl(dimethyl)silyl]oxy}pyrrolidin-1-yl]-2-oxo-2H-1,3′-bipyridin-4-yl 4-bromobenzenesulfonate

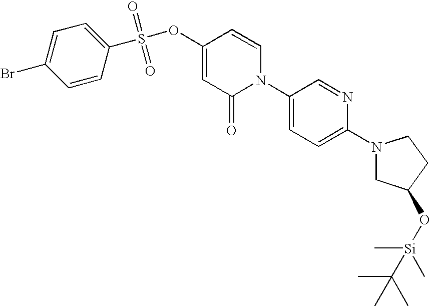

6′-[(3R)-3-{[tert-Butyl(dimethyl)silyl]oxy}pyrrolidin-1-yl]-4-hydroxy-2H-1,3′-bipyridin-2-one from Preparation 46 (6.185 g, 15.96 mmol) was suspended in DCM (100 ml), triethylamine (4.45 ml, 31.9 mmol) was added in one portion and the mixture stirred for 20 minutes at r.t. to give a homogenous solution. The mixture was cooled to 0° C. and 4-bromobenzenesulfonyl chloride (4.280 g, 16.8 mmol) was added in one portion, reaction exothermed to 5° C. The mixture was stirred overnight at r.t. LC-MS indicated reaction complete. 10% aqueous potassium carbonate (200 ml) was added and the mixture stirred for 30 minutes. The DCM layer was separated washed with water (3×100 ml) then brine (100 ml), dried (MgSO4) then evaporated to give the title compound as a white amorphous powder (7.60 g, 78%) ¹H-NMR (400 MHz, d₆-DMSO) δ ppm 0.08 (s, 6H), 0.81 (s, 9H), 1.80-1.92 (m, 1H), 2.05-2.15 (m, 1H), 3.21-3.30 (m, 1H), 3.40-3.49 (m, 2H), 3.57-3.63 (m, 1H), 4.57-4.61 (m, 1H), 6.18 (d, 2H), 6.52 (d, 1H), 7.48 (d, 1H), 7.75 (d, 1H), 7.98 (s, 4H), 8.0 (s, 1H) LRMS m/z (ESI) 606/608 [MH⁺]

### Preparation 48

### 4-(Methylthio)pyridin-2(1H)-one

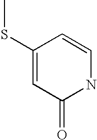

4-(Methylthio)pyridine 1-oxide (Jpn. Kokai Tokkyo Koho (1990), 5 pp. CODEN: JKXXAF JP 02124872, 100 mg, 0.708 mmol) was added to acetic anhydride (72.3 mg, 0.708 mmol) and refluxed for 6 hours, the acetic anhydride was then evaporated and the residue refluxed with methanol (5 ml) for 3 hours, the mixture was then evaporated and the residue triturated with MTBE (3×1 ml) to give the title compound as an amorphous dark brown solid (99 mg, 99%) ¹H-NMR (400 MHz, CD₃OD) [symbol] ppm 2.41 (s, 3H), 6.25 (d, 2H), 7.21 (s, 1H).

### Preparation 49

### 6′-[(3R)-3-Hydroxypyrrolidin-1-yl]-4-(methylthio)-2H-1,3′-bipyridin-2-one

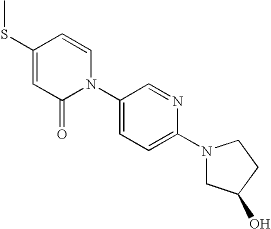

Prepared according to Preparation 1 using 4-(Methylthio)pyridin-2(1H)-one from Preparation 48 and (3R)-1-(5-Iodopyridin-2-yl)pyrrolidin-3-ol (WO2004039780) to give the title compound as a dark brown amorphous solid which was used without further purification.

### Preparation 50

### 6′-[(3R)-3-{[tert-Butyl(dimethyl)silyl]oxy}pyrrolidin-1-yl]-4-(methylthio)-2H-1,3′-bipyridin-2-one

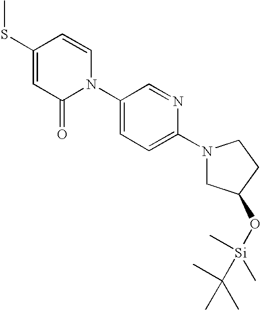

Prepared according to Preparation 45 using 6′-[(3R)-3-Hydroxypyrrolidin-1-yl]-4-(methylthio)-2H-1,3′-bipyridin-2-one from Preparation 49, triethylamine as base to give the title compound as a grey solid. ¹H-NMR (400 MHz, d₆-DMSO) δ ppm 0.09 (s, 15H), 1.90-2.05 (m, 1H), 2.09-2.20 (m, 1H), 1.67 (s, 3H), 2.59 (d, 1H), 2.74-2.80 (m, 2H), 2.82 (dd, 1H), 3.79-3.87 (m, 1H), 5.51 (s, 1H), 5.55 (d, 1H), 5.77 (d, 1H), 6.60 (d, 1H), 6.72 (d, 1H), 7.20 (s, 1H) LC-MS RT=3.34 min m/z (APCI & ESI) 418 [MH⁺] (6 min acidic run)

### Preparation 51

### 6′-[(3R)-3-{[tert-Butyl(dimethyl)silyl]oxy}pyrrolidin-1-yl]-4-(methanesulfonyl)-2H-1,3′-bipyridin-2-one

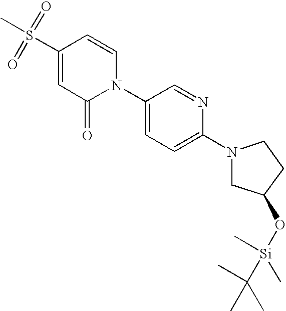

An aqueous mixture of 30% H₂O₂ (12.2 ml, 120 mmol) and a 0.2M solution of NaHCO₃ (408 ml) previously prepared at 0° C. was added to a vigorously stirred solution of 6′-[(3R)-3-{[tert-Butyl(dim ethyl)silyl]oxy}pyrrolidin-1-yl]-4-(methylthio)-2H-1,3′-bipyridin-2-one from Preparation 50 (10.0 g, 23.944 mmol) in acetonitrile (552 ml) in a single addition. The mixture was vigorously stirred with a mechanical overhead stirrer for 8 hours. Reaction complete by LC-MS. Reaction mixture was poured in brine (1000 ml) the mixture was then extracted with EtOAc (2×500 ml). The extracts were then evaporated to give a glassy green amorphous solid which was purified on an ISCO 330 g column eluting from 100% DCM 100% EtOAC over 10 CV to give the title compound as a solid (9.00 g, 83%) ¹H-NMR (400 MHz, CD₃OD) δ ppm 0.12 (d, 6H), 0.90 (s, 9H), 1.93-2.02 (m, 1H), 2.18-2.20 (m, 1H), 3.19 (s, 3H), 3.39 (d, 1H), 3.57-3.60 (m, 2H), 3.60-3.67 (m, 1H), 4.78-4.83 (m, 1H), 6.59 (d, 1H), 6.79 (d, 1H), 7.19 (s, 1H), 7.58 (d, 1H), 7.83 (d, 1H), 8.04 (s, 1H)

### Preparation 52

### 6′-{(3S)-3-[(tert-Butoxycarbonyl)(methyl)amino]pyrrolidin-1-yl}-2-oxo-2H-1,3′-bipyridinyl-4-yl trifluoromethanesulfonate

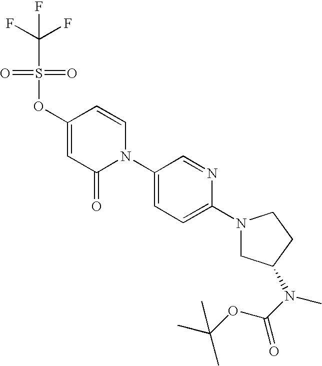

To a cold (−50° C.) solution of tert-Butyl[(3S)-1-(4-hydroxy-2-oxo-2H-1,3′-bipyridin-6′-yl)pyrrolidin-3-yl]methylcarbamate from Preparation 24 (100 mg, 0.259 mmol) in DCM (2 mL) was added triethylamine (57.7 uL, 0.414 mmol) followed by addition of trifluoromethanesulphonic anhydride (65.3 uL, 0.388 mmol). The resulting mixture was stirred at −30° C. under nitrogen for 2 h. The reaction mixture was then poured into ice/water mixture (5 mL) and the products were extracted with DCM (2×5 mL). The combined organic extracts were washed with water (2×5 mL), dried over a separating phase cartridge and evaporated to give the title compound as a yellow oil (115 mg, 85%) ¹H-NMR (400 MHz, CDCl₃) δ ppm 1.48 (s, 9H), 2.06-2.35 (m, 2H), 2.84 (s, 3H), 3.37-3.57 (m, 2H), 3.68-3.79 (m, 2H), 4.88 (br. s, 1H), 6.28 (dd, 1H), 6.49 (d, 1H), 6.57 (d, 1H), 7.44 (d, 1H), 7.58 (dd, 1H), 8.11 (d, 1H). LRMS m/z (APCI & ESI) 519 [MH⁺]

### Preparation 54

### N-{(3S)-1-[4-(Benzyloxy)-2-oxo-2H-1,3′-bipyridin-6′-yl]pyrrolidin-3-yl}-4-chlorobutanamide

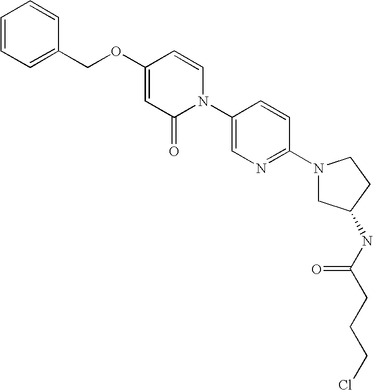

4-Chlorobutyryl chloride (0.081 ml, 0.73 mmol) was added to a mixture of 6′-[(3S)-3-aminopyrrolidin-1-yl]-4-(benzyloxy)-2H-1,3′-bipyridin-2-one Example 55 (239 mg, 0.66 mmol) and triethylamine (0.092 ml, 0.66 mmol) in tetrahydrofuran (5 ml), and the opaque reaction mixture was stirred at r.t. for 2 hours. MS showed product mass ion with no SM remaining. Solvents were evaporated then the residue was dissolved in DCM (20 mL) and washed with water (30 mL) then brine (20 mL) before drying the organics (MgSO₄) filtering and evaporating in vacuo to give the title compound as a yellow powder which was used without further purification (447 mg). LRMS m/z (ESI) 467 [MH+]

It is to be understood that the foregoing description is exemplary and explanatory in nature, and is intended to illustrate the invention and its preferred embodiments. Through routine experimentation, the artisan will recognize apparent modifications and variations that may be made without departing from the spirit of the invention. Thus, the invention is intended to be defined not by the above description, but by the following claims and their equivalents.

1. A compound of formula (I) 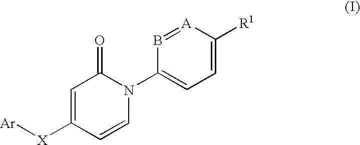
wherein:
X is CH₂CH₂, CH₂O or OCH₂;
A and B are each independently CH or N, with the proviso that 1 or both of A and B is N;
Ar is phenyl optionally substituted by 1 or 2 substituents independently selected from F and Cl;
R¹ is a saturated 4- to 9-membered heterocyclic ring system containing 1 or 2 ring N atoms,
which ring system may incorporate spiro-, fused or bridged rings,
which is attached to the “ABCCHCHC” ring via a N atom,
which ring system is optionally substituted by one or more substituents independently selected from ═O, R⁹, OH, C(O)C₁-C₅ alkyl, C(O)C₃-C₅ cycloalkyl, C(O)OC₁-C₅ alkyl, NR⁸R⁷, NR⁸C(O)R⁹, NR⁸C(O)OR⁹, O(C₁-C₅ alkyl) or O(C₃-C₅ cycloalkyl);
R⁸ and R⁷ are each independently H, C₁-C₅ alkyl or C₃-C₅ cycloalkyl;
or R⁶ and R⁷ can be taken together with the N atom to which they are attached to form a 4- to 7-membered saturated ring, optionally substituted by ═O;
R⁸ is H, C₁-C₅ alkyl or C₃-C₅ cycloalkyl;
R⁹ is C₁-C₅ alkyl or C₃-C₅ cycloalkyl, each of which is optionally substituted with one or more fluorine atoms;
or a pharmaceutically acceptable salt, solvate or prodrug thereof.

2. A compound according to claim 1 wherein X—Ar is OCH₂—Ar, or a pharmaceutically acceptable salt, solvate or prodrug thereof.

3. A compound according to claim 1 or 2 wherein A is N and B is CH or N, or a pharmaceutically acceptable salt, solvate or prodrug thereof.

4. A compound according to claim 3 wherein Ar is phenyl, fluorophenyl or chlorophenyl, or a pharmaceutically acceptable salt, solvate or prodrug thereof.

5. A compound according to claim 4 wherein R¹ is piperidine, pyrrolidine, piperazine, octahydro-pyrrolo[3,4-c]pyrrole, octahydro-pyrrolo[3,4-b]pyrrole, or octahydro-pyrrolo[3,4-c]pyridine,
attached to the “ABCCHCHC” ring via a N atom,
which ring system is optionally substituted by one or more substituents independently selected from ═O, C₁-C₅ alkyl, C₃-C₅ cycloalkyl, OH, C(O)C₁-C₅ alkyl, C(O)C₃-C₅ cycloalkyl, C(O)OC₁-C₅ alkyl, NR⁶R⁷, NR⁸C(O)R⁹, NR⁸C(O)OR⁹, O(C₁-C₅ alkyl) or O(C₃-C₅ cycloalkyl),
or a pharmaceutically acceptable salt, solvate or prodrug thereof.

6. A compound according to claim 5 wherein A is N and B is CH, or a pharmaceutically acceptable salt, solvate or prodrug thereof.

7. A compound according to claim 6 wherein Ar is phenyl, 4-chlorophenyl or 4-fluorophenyl, or a pharmaceutically acceptable salt, solvate or prodrug thereof.

8. A compound according to claim 7 wherein R¹ is piperidine, pyrrolidine, piperazine, octahydro-pyrrolo[3,4-c]pyrrole, octahydro-pyrrolo[3,4-b]pyrrole, or octahydro-pyrrolo[3,4-c]pyridine,
attached to the “ABCCHCHC” ring via a N atom,
which ring system is optionally substituted by one or more substituents independently selected from OH, OMe, OEt, Me, Et, NH₂, NHMe, NMe₂, NMeC(O)Me and C(O)Me,
or a pharmaceutically acceptable salt, solvate or prodrug thereof.

9. A compound according to claim 8 wherein Ar is phenyl, or a pharmaceutically acceptable salt, solvate or prodrug thereof.

10. A compound according to claim 9 wherein R¹ is piperidine, pyrrolidine, piperazine, octahydro-pyrrolo[3,4-c]pyrrole, octahydro-pyrrolo[3,4-b]pyrrole, or octahydro-pyrrolo[3,4-c]pyridine,
attached to the “ABCCHCHC” ring via a N atom,
which ring system is optionally substituted by one or more substituents independently selected from NHMe, NMe₂, OH, NH₂, Me and Et,
or a pharmaceutically acceptable salt, solvate or prodrug thereof.

11. A compound according to claim 10 wherein R¹ is piperidine, pyrrolidine, piperazine, octahydro-pyrrolo[3,4-c]pyrrole, octahydro-pyrrolo[3,4-b]pyrrole, or octahydro-pyrrolo[3,4-c]pyridine,
attached to the “ABCCHCHC” ring via a N atom,
which ring system is optionally substituted by one or more substituents independently selected from NHMe, NMe₂, OH, NH₂, Me and Et,
or a pharmaceutically acceptable salt, solvate or prodrug thereof.

12. A compound according to claim 11 wherein R¹ is selected from one of the following groups: 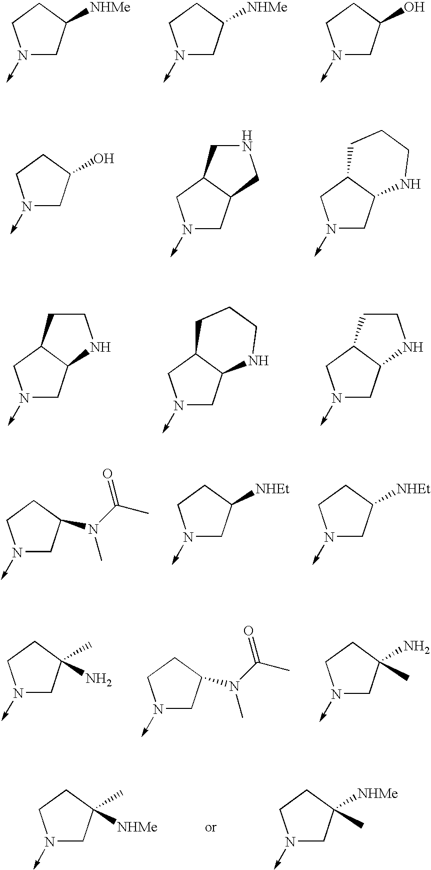
or a pharmaceutically acceptable salt, solvate or prodrug thereof.

13. (canceled)

14. A compound according to claim 1 of formula 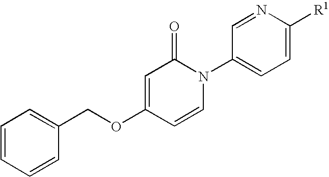
wherein R¹ is selected from 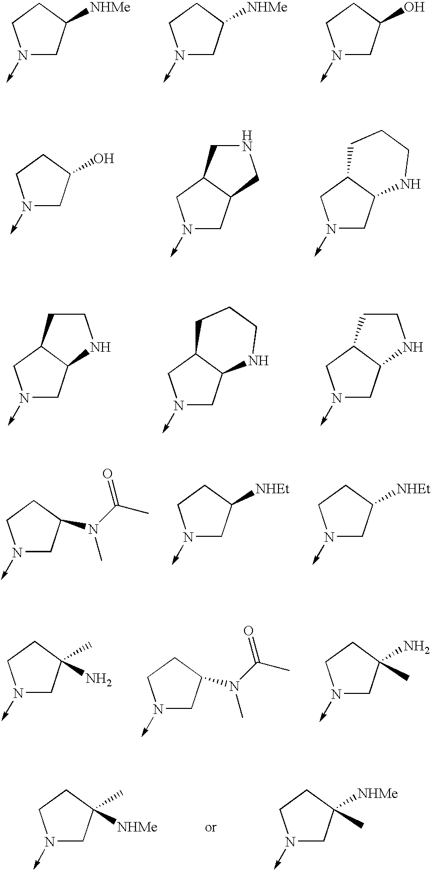
or a pharmaceutically acceptable salt, solvate or prodrug thereof.

15. (canceled)

16. A compound according to claim 1 selected from the group consisting of
6′ (3-Amino-3-methylpyrrolidin-1-yl)-4-(benzyloxy)-2H-1,3′-bipyridin-2-one;
4-(Benzyloxy)-6′-[3-methyl-3-(methylamino)pyrrolidin-1-yl]-2H-1,3′-bipyridin-2-one;
4-(Benzyloxy)-6′-[(3R)-3-(ethylamino)pyrrolidin-1-yl]-2H-1,3′-bipyridin-2-one; and
N-{(3S)-1-[4 (Benzyloxy)-2-oxo-2H-1,3′-bipyridin-6′-yl]pyrrolidin-3-yl}-N-methylacetamide;
or a pharmaceutically acceptable salt, solvate or prodrug thereof.

17. (canceled)

18. A pharmaceutical composition comprising a compound, pharmaceutically acceptable salt, solvate or prodrug thereof according to claim 1, and a pharmaceutically acceptable diluent or carrier.

19-22. (canceled)

23. A method for treating or preventing a disease or condition selected from eating disorders, weight loss or control, obesity, depression, atypical depression, bipolar disorders, psychoses, schizophrenia, behavioral addictions, suppression of reward-related behaviors, substance abuse, addictive disorders, impulsivity, alcoholism, tobacco abuse, dementia, sexual dysfunction in males, seizure disorders, epilepsy, inflammation, gastrointestinal disorders, attention deficit disorder, Parkinson's disease, type II diabetes, premenstrual syndrome or late luteal phase syndrome, migraines, panic disorder, anxiety, post-traumatic syndrome, social phobia, cognitive impairment in non-demented individuals, non-amnestic mild cognitive impairment, post operative cognitive decline, disorders associated with impulsive behaviours, adult personality disorders, diseases associated with impulsive behaviours, and impulse control disorders, obsessive compulsive disorder, chronic fatigue syndrome, premature ejaculation, sexual dysfunction in females, disorders of sleep, autism, mutism, neurodegenerative movement disorders, spinal cord injury, damage of the central nervous system, stroke, neurodegenerative diseases or toxic or infective CNS diseases, cardiovascular disorders which comprises the step of administering to an animal in need thereof a therapeutically effective amount of a MCHR1 antagonist according to claim 1.

24. A method for promoting weight loss, or treatment of obesity and related eating disorders which comprises the step of administering to an animal in need thereof a therapeutically effective amount of a MCHR1 antagonist according to claim 1.
